(19) United States
(12) Patent Application Publication
(10) Pub. No.: US 2018/0092825 A1
(43) Pub. Date: Apr. 5, 2018

(54) HAIR CARE COMPOSITIONS COMPRISING A GEL MATRIX AND GLYCERIDE COPOLYMERS

(71) Applicant: The Procter & Gamble Company, Cincinnati, OH (US)

(72) Inventors: Qing STELLA, Cincinnati, OH (US); Beth Ann SCHUBERT, Maineville, OH (US); Luke Andrew ZANNONI, West Chester, OH (US); Michael Stephen MAILE, Maineville, OH (US); William Richard MUELLER, Cincinnati, OH (US)

(21) Appl. No.: 15/698,868

(22) Filed: Sep. 8, 2017

Related U.S. Application Data
(60) Provisional application No. 62/402,444, filed on Sep. 30, 2016.

(51) Int. Cl. A61K 8/85; A61K 8/02; A61K 8/04; A61K 8/34; C08G 63/00; A61Q 5/12

(52) U.S. Cl. CPC A61K 8/85; A61K 8/0241; A61Q 5/12; A61K 8/342; C08G 63/00; A61K 8/042

(57) ABSTRACT

Disclosed are hair care compositions, such as conditioners, containing certain glyceride copolymers; and a gel matrix phase comprising one or more high melting point fatty compounds, a cationic surfactant system an aqueous carrier. The oligomers provide hair benefits, for example hair conditioning benefits. Also disclosed are methods of using the hair care compositions.

### FIELD OF THE INVENTION

The present invention relates to a hair care composition containing a gel matrix and certain glyceride copolymers, and methods of using the same.

### BACKGROUND OF THE INVENTION

Human hair becomes soiled due to its contact with the surrounding environment and from the sebum secreted by the scalp. The soiling of hair causes it to have a dirty feel and an unattractive appearance.

Shampooing cleans the hair by removing excess soil and sebum. However, shampooing can leave the hair in a wet, tangled, and unmanageable state. Once the hair dries, it is often left in a dry, rough, lusterless, or frizzy condition due to removal of the hair's natural oils.

A variety of approaches have been developed to alleviate these after-shampoo problems. One approach is the application of conditioner after shampooing.

In order to provide hair conditioning benefits after shampooing, a wide variety of conditioning actives have been proposed. These conditioning agents are known to enhance hair shine and provide moisturizing, softness, and static control to the hair. However, such components can also provide stickiness, greasy, or waxy feeling, particularly when the hair is dried. Additionally, the rising costs of silicone and the petroleum based nature of silicone have minimized silicone's desirability as a conditioning active.

Based on the foregoing, there is a need for a conditioning active which can provide conditioning benefits to hair and can replace, or be used in combination with silicone, or other conditioning actives, to maximize the conditioning activity of hair care compositions. Additionally, there is a desire to find a conditioning active which can be derived from a natural source, thereby providing a conditioning active derived from a renewable resource. There is also a desire to find a conditioning active that is derived from a natural source.

### SUMMARY OF THE INVENTION

The present invention relates to hair care compositions as well as methods of making and using same. Such hair care compositions contain certain glyceride copolymers that have the required viscosity and lubricity. Thus, such species of glyceride copolymers provide beneficial conditioning performance and formulability.

In one aspect, the present invention is directed to a hair care composition comprising: (a) from about 0.05% to about 15%, by weight of said hair care composition, of one or more of the glyceride copolymers described below; and (b) a gel matrix phase comprising: (i) from about 0.1% to about 20% of one or more high melting point fatty compounds, by weight of said hair care composition; (ii) from about 0.1% to about 10% of a cationic surfactant system, by weight of said hair care composition; and (iii) at least about 20% of an aqueous carrier, by weight of said hair care composition.

These and other features, aspects, and advantages of the present invention will become evident to those skilled in the art from a reading of the present disclosure.

### DETAILED DESCRIPTION OF THE INVENTION

### Definitions

As used herein, “natural oil”, “natural feedstocks,” or “natural oil feedstocks” refers to oils obtained from plants or animal sources. The term “natural oil” includes natural oil derivatives, unless otherwise indicated. The terms also include modified plant or animal sources (e.g., genetically modified plant or animal sources), and derivatives produced or modified by fermentation or enzymatic processes, unless indicated otherwise. Examples of natural oils include, but are not limited to, vegetable oils, algae oils, fish oils, animal fats, tall oils, derivatives of these oils, combinations of any of these oils, and the like. Representative non-limiting examples of vegetable oils include low erucic acid rapeseed oil (canola oil), high erucic acid rapeseed oil, coconut oil, corn oil, cottonseed oil, olive oil, palm oil, peanut oil, safflower oil, sesame oil, soybean oil, sunflower oil, linseed oil, palm kernel oil, tung oil, jatropha oil, mustard seed oil, pennycress oil, camelina oil, hempseed oil, and castor oil. Representative non-limiting examples of animal fats include lard, tallow, poultry fat, yellow grease, and fish oil. Tall oils are by-products of wood pulp manufacture. In some embodiments, the natural oil or natural oil feedstock comprises one or more unsaturated glycerides (e.g., unsaturated triglycerides). In some such embodiments, the natural oil comprises at least 50% by weight, or at least 60% by weight, or at least 70% by weight, or at least 80% by weight, or at least 90% by weight, or at least 95% by weight, or at least 97% by weight, or at least 99% by weight of one or more unsaturated triglycerides, based on the total weight of the natural oil.

The term “natural oil glyceride” refers to a glyceryl ester of a fatty acid obtained from a natural oil. Such glycerides include monoacylglycerides, diacylglycerides, and triacylglyceriedes (triglycerides). In some embodiments, the natural oil glycerides are triglycerides. Analogously, the term “unsaturated natural oil glyceride” refers to natural oil glycerides, wherein at least one of its fatty acid residues contains unsaturation. For example, a glyceride of oleic acid is an unsaturated natural oil glyceride. The term “unsaturated alkenylized natural oil glyceride” refers to an unsaturated natural oil glyceride (as defined above) that is derivatized via a metathesis reaction with a sort-chain olefin (as defined below). In some cases, olefinizing process shortens one or more of the fatty acid chains in the compound. For example, a glyceride of 9-decenoic acid is an unsaturated alkenylized natural oil glyceride. Similarly, butenylized (e.g., with 1-butene and/or 2-butene) canola oil is a natural oil glyceride that has been modified via metathesis to contain some short-chain unsaturated C₁₀₋₁₅ ester groups.

The term “natural oil derivatives” refers to derivatives thereof derived from natural oil. The methods used to form these natural oil derivatives may include one or more of addition, neutralization, overbasing, saponification, transesterification, interesterification, esterification, amidation, hydrogenation, isomerization, oxidation, alkylation, acylation, sulfurization, sulfonation, rearrangement, reduction, fermentation, pyrolysis, hydrolysis, liquefaction, anaerobic digestion, hydrothermal processing, gasification or a combination of two or more thereof. Examples of natural derivatives thereof may include carboxylic acids, gums, phospholipids, soapstock, acidulated soapstock, distillate or distillate sludge, fatty acids, fatty acid esters, as well as hydroxy substituted variations thereof, including unsaturated polyol esters. In some embodiments, the natural oil derivative may comprise an unsaturated carboxylic acid having from about 5 to about 30 carbon atoms, having one or more carbon-carbon double bonds in the hydrocarbon (alkene) chain. The natural oil derivative may also comprise an unsaturated fatty acid alkyl (e.g., methyl) ester derived from a glyceride of natural oil. For example, the natural oil derivative may be a fatty acid methyl ester (“FAME”) derived from the glyceride of the natural oil. In some embodiments, a feedstock includes canola or soybean oil, as a non-limiting example, refined, bleached, and deodorized oil (i.e., RBD soybean oil).

As used herein, the term “unsaturated polyol ester” refers to a compound having two or more hydroxyl groups wherein at least one of the hydroxyl groups is in the form of an ester and wherein the ester has an organic group including at least one carbon-carbon double bond.

The term “oligomeric glyceride moiety” is a moiety comprising two or more, in one aspect, up to 20, in another aspect, up to 10 constitutional units formed via olefin metathesis from natural oil glycerides and/or alkenylized natural oil glycerides.

The term “free hydrocarbon” refers to any one or combination of unsaturated or saturated straight, branched, or cyclic hydrocarbons in the C₂₋₃₀ range.

The term “metathesis monomer” refers to a single entity that is the product of an olefin metathesis reaction which comprises a molecule of a compound with one or more carbon-carbon double bonds which has undergone an alkylidene unit interchange via one or more of the carbon-carbon double bonds either within the same molecule (intramolecular metathesis) and/or with a molecule of another compound containing one or more carbon-carbon double bonds such as an olefin (intermolecular metathesis). In some embodiments, the term refers to a triglyceride or other unsaturated polyol ester that has not yet undergone an alkylidene unit interchange but contains at least one C₄₋₁₇ ester having a carbon-carbon double bond in the “omega minus n” position, where n=0, 1, 2, 3, 4, 5, or 6 and where the ester moiety has at least n+3 carbon atoms.

The term “metathesis dimer” refers to the product of a metathesis reaction wherein two reactant compounds, which can be the same or different and each with one or more carbon-carbon double bonds, are bonded together via one or more of the carbon-carbon double bonds in each of the reactant compounds as a result of the metathesis reaction.

The term “metathesis trimer” refers to the product of one or more metathesis reactions wherein three molecules of two or more reactant compounds, which can be the same or different and each with one or more carbon-carbon double bonds, are bonded together via one or more of the carbon-carbon double bonds in each of the reactant compounds as a result of the one or more metathesis reactions, the trimer containing three bonded groups derived from the reactant compounds.

The term “metathesis tetramer” refers to the product of one or more metathesis reactions wherein four molecules of two or more reactant compounds, which can be the same or different and each with one or more carbon-carbon double bonds, are bonded together via one or more of the carbon-carbon double bonds in each of the reactant compounds as a result of the one or more metathesis reactions, the tetramer containing four bonded groups derived from the reactant compounds.

The term “metathesis pentamer” refers to the product of one or more metathesis reactions wherein five molecules of two or more reactant compounds, which can be the same or different and each with one or more carbon-carbon double bonds, are bonded together via one or more of the carbon-carbon double bonds in each of the reactant compounds as a result of the one or more metathesis reactions, the pentamer containing five bonded groups derived from the reactant compounds.

The term “metathesis hexamer” refers to the product of one or more metathesis reactions wherein six molecules of two or more reactant compounds, which can be the same or different and each with one or more carbon-carbon double bonds, are bonded together via one or more of the carbon-carbon double bonds in each of the reactant compounds as a result of the one or more metathesis reactions, the hexamer containing six bonded groups derived from the reactant compounds.

The term “metathesis heptamer” refers to the product of one or more metathesis reactions wherein seven molecules of two or more reactant compounds, which can be the same or different and each with one or more carbon-carbon double bonds, are bonded together via one or more of the carbon-carbon double bonds in each of the reactant compounds as a result of the one or more metathesis reactions, the heptamer containing seven bonded groups derived from the reactant compounds.

The term “metathesis octamer” refers to the product of one or more metathesis reactions wherein eight molecules of two or more reactant compounds, which can be the same or different and each with one or more carbon-carbon double bonds, are bonded together via one or more of the carbon-carbon double bonds in each of the reactant compounds as a result of the one or more metathesis reactions, the octamer containing eight bonded groups derived from the reactant compounds.

The term “metathesis nonamer” refers to the product of one or more metathesis reactions wherein nine molecules of two or more reactant compounds, which can be the same or different and each with one or more carbon-carbon double bonds, are bonded together via one or more of the carbon-carbon double bonds in each of the reactant compounds as a result of the one or more metathesis reactions, the nonamer containing nine bonded groups derived from the reactant compounds.

The term “metathesis decamer” refers to the product of one or more metathesis reactions wherein ten molecules of two or more reactant compounds, which can be the same or different and each with one or more carbon-carbon double bonds, are bonded together via one or more of the carbon-carbon double bonds in each of the reactant compounds as a result of the one or more metathesis reactions, the decamer containing ten bonded groups derived from the reactant compounds.

The term “metathesis oligomer” refers to the product of one or more metathesis reactions wherein two or more molecules (e.g., 2 to about 10, or 2 to about 4) of two or more reactant compounds, which can be the same or different and each with one or more carbon-carbon double bonds, are bonded together via one or more of the carbon-carbon double bonds in each of the reactant compounds as a result of the one or more metathesis reactions, the oligomer containing a few (e.g., 2 to about 10, or 2 to about 4) bonded groups derived from the reactant compounds. In some embodiments, the term “metathesis oligomer” may include metathesis reactions wherein greater than ten molecules of two or more reactant compounds, which can be the same or different and each with one or more carbon-carbon double bonds, are bonded together via one or more of the carbon-carbon double bonds in each of the reactant compounds as a result of the one or more metathesis reactions, the oligomer containing greater than ten bonded groups derived from the reactant compounds.

As used herein, “metathesis” refers to olefin metathesis. As used herein, “metathesis catalyst” includes any catalyst or catalyst system that catalyzes an olefin metathesis reaction.

As used herein, “metathesize” or “metathesizing” refer to the reacting of a feedstock in the presence of a metathesis catalyst to form a “metathesized product” comprising new olefinic compounds, i.e., “metathesized” compounds. Metathesizing is not limited to any particular type of olefin metathesis, and may refer to cross-metathesis (i.e., co-metathesis), self-metathesis, ring-opening metathesis, ring-opening metathesis polymerizations (“ROMP”), ring-closing metathesis (“RCM”), and acyclic diene metathesis (“ADMET”). In some embodiments, metathesizing refers to reacting two triglycerides present in a natural feedstock (self-metathesis) in the presence of a metathesis catalyst, wherein each triglyceride has an unsaturated carbon-carbon double bond, thereby forming a new mixture of olefins and esters which may include a triglyceride dimer. Such triglyceride dimers may have more than one olefinic bond, thus higher oligomers also may form. These higher order oligomers may comprise one or more of: metathesis monomers, metathesis dimers, metathesis trimers, metathesis tetramers, metathesis pentamers, and higher order metathesis oligomers (e.g., metathesis hexamers, metathesis, metathesis heptamers, metathesis octamers, metathesis nonamers, metathesis decamers, and higher than metathesis decamers and above). Additionally, in some other embodiments, metathesizing may refer to reacting an olefin, such as ethylene, and a triglyceride in a natural feedstock having at least one unsaturated carbon-carbon double bond, thereby forming new olefinic molecules as well as new ester molecules (cross-metathesis).

As used herein, the term “olefinized natural polyol ester and/or olefinized synthetic polyol ester” refers to the product produced by metathesizing a natural and/or synthetic polyol ester with a C₂₋₁₄ olefin, preferably C₂₋₆ olefin, more preferably C₃₋₄ olefin, and mixtures and isomers thereof.

As used herein, “olefin” or “olefins” refer to compounds having at least one unsaturated carbon-carbon double bond. In certain embodiments, the term “olefins” refers to a group of unsaturated carbon-carbon double bond compounds with different carbon lengths. Unless noted otherwise, the terms “olefin” or “olefins” encompasses “polyunsaturated olefins” or “polyolefins,” which have more than one carbon-carbon double bond. As used herein, the term “monounsaturated olefins” or “mono-olefins” refers to compounds having only one carbon-carbon double bond. A compound having a terminal carbon-carbon double bond can be referred to as a “terminal olefin” or an “alpha-olefin,” while an olefin having a non-terminal carbon-carbon double bond can be referred to as an “internal olefin.” In some embodiments, the alpha-olefin is a terminal alkene, which is an alkene (as defined below) having a terminal carbon-carbon double bond. Additional carbon-carbon double bonds can be present.

The number of carbon atoms in any group or compound can be represented by the terms: “C_{z}”, which refers to a group of compound having z carbon atoms; and “C_{x-y}”, which refers to a group or compound containing from x to y, inclusive, carbon atoms. For example, “C₁₋₆ alkyl” represents an alkyl chain having from 1 to 6 carbon atoms and, for example, includes, but is not limited to, methyl, ethyl, n-propyl, isopropyl, isobutyl, n-butyl, sec-butyl, tert-butyl, isopentyl, n-pentyl, neopentyl, and n-hexyl. As a further example, a “C₄₋₁₀ alkene” refers to an alkene molecule having from 4 to 10 carbon atoms, and, for example, includes, but is not limited to, 1-butene, 2-butene, isobutene, 1-pentene, 1-hexene, 3-hexene, 1-heptene, 3-heptene, 1-octene, 4-octene, 1-nonene, 4-nonene, and 1-decene.

As used herein, the terms “short-chain alkene” or “short-chain olefin” refer to any one or combination of unsaturated straight, branched, or cyclic hydrocarbons in the C₂₋₁₄ range, or the C₂₋₁₂ range, or the C₂₋₁₀ range, or the C₂₋₈ range. Such olefins include alpha-olefins, wherein the unsaturated carbon-carbon bond is present at one end of the compound. Such olefins also include dienes or trienes. Such olefins also include internal olefins. Examples of short-chain alkenes in the C₂₋₆ range include, but are not limited to: ethylene, propylene, 1-butene, 2-butene, isobutene, 1-pentene, 2-pentene, 2-methyl-1-butene, 2-methyl-2-butene, 3-methyl-1-butene, cyclopentene, 1,4-pentadiene, 1-hexene, 2-hexene, 3-hexene, 2-methyl-1-pentene, 3-methyl-1-pentene, 4-methyl-1-pentene, 2-methyl-2-pentene, 3-methyl-2-pentene, 4-methyl-2-pentene, 2-methyl-3-pentene, and cyclohexene. Non-limiting examples of short-chain alkenes in the C₇₋₉ range include 1,4-heptadiene, 1-heptene, 3,6-nonadiene, 3-nonene, 1,4,7-octatriene. In certain embodiments, it is preferable to use a mixture of olefins, the mixture comprising linear and branched low-molecular-weight olefins in the C₄₋₁₀ range. In some embodiments, it may be preferable to use a mixture of linear and branched C₄ olefins (i.e., combinations of: 1-butene, 2-butene, and/or isobutene). In other embodiments, a higher range of C₁₁₋₁₄ may be used.

As used herein, “alkyl” refers to a straight or branched chain saturated hydrocarbon having 1 to 30 carbon atoms, which may be optionally substituted, as herein further described, with multiple degrees of substitution being allowed. Examples of “alkyl,” as used herein, include, but are not limited to, methyl, ethyl, n-propyl, isopropyl, isobutyl, n-butyl, sec-butyl, tert-butyl, isopentyl, n-pentyl, neopentyl, n-hexyl, and 2-ethylhexyl. The number of carbon atoms in an alkyl group is represented by the phrase “C_{x-y} alkyl,” which refers to an alkyl group, as herein defined, containing from x to y, inclusive, carbon atoms. Thus, “C₁₋₆ alkyl” represents an alkyl chain having from 1 to 6 carbon atoms and, for example, includes, but is not limited to, methyl, ethyl, n-propyl, isopropyl, isobutyl, n-butyl, sec-butyl, tert-butyl, isopentyl, n-pentyl, neopentyl, and n-hexyl. In some instances, the “alkyl” group can be divalent, in which case the group can alternatively be referred to as an “alkylene” group.

As used herein, “alkenyl” refers to a straight or branched chain non-aromatic hydrocarbon having 2 to 30 carbon atoms and having one or more carbon-carbon double bonds, which may be optionally substituted, as herein further described, with multiple degrees of substitution being allowed. Examples of “alkenyl,” as used herein, include, but are not limited to, ethenyl, 2-propenyl, 2-butenyl, and 3-butenyl. The number of carbon atoms in an alkenyl group is represented by the phrase “C_{x-y} alkenyl,” which refers to an alkenyl group, as herein defined, containing from x to y, inclusive, carbon atoms. Thus, “C₂₋₆ alkenyl” represents an alkenyl chain having from 2 to 6 carbon atoms and, for example, includes, but is not limited to, ethenyl, 2-propenyl, 2-butenyl, and 3-butenyl. In some instances, the “alkenyl” group can be divalent, in which case the group can alternatively be referred to as an “alkenylene” group.

As used herein, “direct bond” refers to an embodiment where the identified moiety is absent from the structure, and is replaced by a bond between other moieties to which it is connected. For example, if the specification or claims recite A-D-E and D is defined as a direct bond, the resulting structure is A-E.

As used herein, “substituted” refers to substitution of one or more hydrogen atoms of the designated moiety with the named substituent or substituents, multiple degrees of substitution being allowed unless otherwise stated, provided that the substitution results in a stable or chemically feasible compound. A stable compound or chemically feasible compound is one in which the chemical structure is not substantially altered when kept at a temperature from about −80° C. to about +40° C., in the absence of moisture or other chemically reactive conditions, for at least a week. As used herein, the phrases “substituted with one or more . . . ” or “substituted one or more times . . . ” refer to a number of substituents that equals from one to the maximum number of substituents possible based on the number of available bonding sites, provided that the above conditions of stability and chemical feasibility are met.

As used herein, the term “polyol” means an organic material comprising at least two hydroxy moieties.

As used herein, the term “C₁₀₋₁₄ unsaturated fatty acid ester” means a fatty acid ester that comprises 10, 11, 12, 13 or 14 carbon atoms, wherein the fatty acid ester chain has at least one carbon-carbon double bond.

In some instances herein, organic compounds are described using the “line structure” methodology, where chemical bonds are indicated by a line, where the carbon atoms are not expressly labeled, and where the hydrogen atoms covalently bound to carbon (or the C—H bonds) are not shown at all. For example, by that convention, the formula

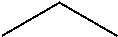

represents n-propane. In some instances herein, a squiggly bond is used to show the compound can have any one of two or more isomers. For example, the structure

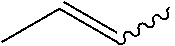

can refer to (E)-2-butene or (Z)-2-butene. The same is true when olefinic structures are drawn that are ambiguous as to which isomer is referred to. For example, CH₃—CH═CH—CH₃ can refer to (E)-2-butene or (Z)-2-butene.

As used herein, the various functional groups represented will be understood to have a point of attachment at the functional group having the hyphen or dash (-) or an asterisk (*). In other words, in the case of —CH₂CH₂CH₃, it will be understood that the point of attachment is the CH₂ group at the far left. If a group is recited without an asterisk or a dash, then the attachment point is indicated by the plain and ordinary meaning of the recited group.

As used herein, multi-atom bivalent species are to be read from left to right. For example, if the specification or claims recite A-D-E and D is defined as —OC(O)—, the resulting group with D replaced is: A-OC(O)-E and not A-C(O)O-E.

As used herein, the articles including “a” and “an” when used in a claim, are understood to mean one or more of what is claimed or described.

As used herein, the terms “include”, “includes” and “including” are meant to be non-limiting.

Unless otherwise noted, all component or composition levels are in reference to the active portion of that component or composition, and are exclusive of impurities, for example, residual solvents or by-products, which may be present in commercially available sources of such components or compositions.

All percentages and ratios are calculated by weight unless otherwise indicated. All percentages and ratios are calculated based on the total composition unless otherwise indicated.

It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

### Compositions and Methods of Use

### Paragraphs (a) Through (ff)

The following compositions, methods of use and treated articles are disclosed:
(a) A composition comprising,
A) a material selected from the group consisting of:
   (i), a first glyceride copolymer, comprising, based on total weight of first glyceride copolymer, from about 3% to about 30%, from about 3% to about 25%, or from about 5% to about 20% C₁₀₋₁₄ unsaturated fatty acid esters; in one aspect, said first glyceride copolymer comprises, based on total weight of first glyceride copolymer, from about 3% to about 30%, from about 3% to about 25%, or from about 3% to about 20% C₁₀₋₁₃ unsaturated fatty acid esters; in one aspect said first glyceride copolymer comprises, based on total weight of first glyceride copolymer, from about 0.1% to about 30%, from about 0.1% to about 25%, from about 0.2% to about 20%, or from about 0.5% to about 15% C₁₀₋₁₁ unsaturated fatty acid esters;
   (ii) a second glyceride copolymer having formula (I):

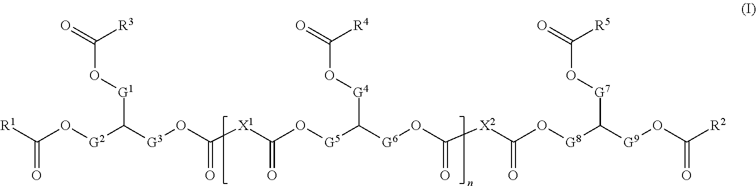

wherein:
   each R¹, R², R³, R⁴, and R⁵ in the second glyceride copolymer is independently selected from the group consisting of an oligomeric glyceride moiety, a C₁₋₂₄ alkyl, a substituted C₁₋₂₄ alkyl wherein the substituent is one or more —OH moieties, a C₂₋₂₄ alkenyl, or a substituted C₂₋₂₄ alkenyl wherein the substituent is one or more —OH moieties; and/or wherein each of the following combinations of moieties may each independently be covalently linked:
      R¹ and R³,
      R² and R⁵,
      R¹ and an adjacent R⁴,
      R² and an adjacent R⁴,
      R³ and an adjacent R⁴,
      R⁵ and an adjacent R⁴, or
      any two adjacent R⁴
   such that the covalently linked moieties form an alkenylene moiety; each X¹ and X² in said second glyceride copolymer is independently selected from the group consisting of a C₁₋₃₂ alkylene, a substituted C₁₋₃₂ alkylene wherein the substituent is one or more —OH moieties, a C₂₋₃₂ alkenylene or a substituted C₂₋₃₂ alkenylene wherein the substituent is one or more —OH moieties;
   two of G¹, G², and G³ are —CH₂—, and one of G¹, G², and G³ is a direct bond;
   for each individual repeat unit in the repeat unit having index n, two of G⁴, G⁵, and G⁶ are —CH₂—, and one of G⁴, G⁵, and G⁶ is a direct bond, and the values G⁴, G⁵, and G⁶ for each individual repeat unit are independently selected from the values of G⁴, G⁵, and G⁶ in other repeating units;
   two of G⁷, G⁸, and G⁹ are —CH₂—, and one of G⁷, G⁸, and G⁹ is a direct bond;
   n is an integer from 3 to 250;
   with the proviso for each of said second glyceride copolymers at least one of R¹, R², R³, and R⁵, and/or at least one R⁴ in one individual repeat unit of said repeat unit having index n, is selected from the group consisting of: 8-nonenyl; 8-decenyl; 8-undecenyl; 8-dodecenyl; 8,11-dodecadienyl; 8,11-tridecadienyl; 8,11-tetradecadienyl; 8,11-pentadecadienyl; 8,11,14-pentadecatrienyl; 8,11,14-hexadecatrienyl; 8,11,14-octadecatrienyl; 9-methyl-8-decenyl; 9-methyl-8-undecenyl; 10-methyl-8-undecenyl; 12-methyl-8,11-tridecadienyl; 12-methyl-8,11-tetradecadienyl; 13-methyl-8,11-tetradecadienyl; 15-methyl-8,11,14-hexadecatrienyl; 15-methyl-8,11,14-heptadecatrienyl; 16-methyl-8,11,14-heptadecatrienyl; 12-tridecenyl; 12-tetradecenyl; 12-pentadecenyl; 12-hexadecenyl; 13-methyl-12-tetradecenyl; 13-methyl-12-pentadecenyl; and 14-methyl-12-pentadecenyl; in one aspect, said second glyceride copolymer comprises based on total weight of second glyceride copolymer, from about 3% to about 30%, from about 3% to about 25%, or from about 5% to about 20% C₉₋₁₃ alkenyl moieties; in one aspect, said second glyceride copolymer comprises, based on total weight of second glyceride copolymer, from about 3% to about 30%, from about 3% to about 25%, or from about 3% to about 20% C₉₋₁₂ alkenyl moieties; in one aspect, said second glyceride copolymer comprises, based on total weight of second glyceride copolymer, from about 0.1% to about 30%, from about 0.1% to about 25%, from about 0.2% to about 20%, or from about 0.5% to about 15% C₉₋₁₀ alkenyl moieties; and
(iii) optionally, a third glyceride copolymer, which comprises constitutional units formed from reacting, in the presence of a metathesis catalyst, one or more compounds from each of the compounds having the following formulas:

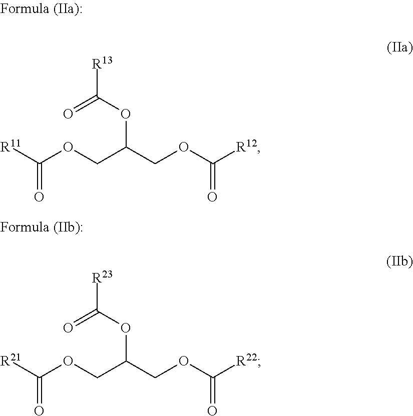

wherein,
   each R¹¹, R¹², and R¹³ is independently a C₁₋₂₄ alkyl, a substituted C₁₋₂₄ alkyl wherein the substituent is one or more —OH moieties, a C₂₋₂₄ alkenyl, or a substituted C₂₋₂₄ alkenyl wherein the substituent is one or more —OH moieties with the proviso that at least one of R¹¹, R¹², and R¹³ is a C₂₋₂₄ alkenyl or a substituted C₂₋₂₄ alkenyl wherein the substituent is one or more —OH moieties; and
   each R²¹, R²², and R²³ is independently a C₁₋₂₄ alkyl, a substituted C₁₋₂₄ alkyl wherein the substituent is one or more —OH moieties, a C₂₋₂₄ alkenyl, or a substituted C₂₋₂₄ alkenyl wherein the substituent is one or more —OH moieties, with the proviso that at least one of R²¹, R²², and R²³ is 8-nonenyl; 8-decenyl; 8-undecenyl; 8-dodecenyl; 8,11-dodecadienyl; 8,11-tridecadienyl; 8,11-tetradecadienyl; 8,11-pentadecadienyl; 8,11,14-pentadecatrienyl; 8,11,14-hexadecatrienyl; 8,11,14-octadecatrienyl; 9-methyl-8-decenyl; 9-methyl-8-undecenyl; 10-methyl-8-undecenyl; 12-methyl-8,11-tridecadienyl; 12-methyl-8,11-tetradecadienyl; 13-methyl-8,11-tetradecadienyl; 15-methyl-8,11,14-hexadecatrienyl; 15-methyl-8,11,14-heptadecatrienyl; 16-methyl-8,11,14-heptadecatrienyl; 12-tridecenyl; 12-tetradecenyl; 12-pentadecenyl; 12-hexadecenyl; 13-methyl-12-tetradecenyl; 13-methyl-12-pentadecenyl; and 14-methyl-12-pentadecenyl;
wherein the number ratio of constitutional units formed from monomer compounds of formula (IIa) to constitutional units formed from monomer compounds of formula (IIb) is no more than 10:1; and
(iv) mixtures thereof; and
B) a gel matrix phase comprising: (i) from about 0.1% to about 20% of one or more high melting point fatty compounds, by weight of said hair care composition; (ii) from about 0.1% to about 10% of a cationic surfactant system, by weight of said hair care composition; and (iii) at least about 20% of an aqueous carrier, by weight of said hair care composition.
   (b) The composition of Paragraph (a) wherein said first, second, and third glyceride copolymers have a weight average molecular weight of from about 4,000 g/mol to about 150,000 g/mol, from about 5,000 g/mol to about 130,000 g/mol, from about 6,000 g/mol to about 100,000 g/mol, from about 7,000 g/mol to about 50,000 g/mol, from about 8,000 g/mol to about 30,000 g/mol, or from about 8,000 g/mol to about 20,000 g/mol.
   (c) The composition according to Paragraphs (a) through (b) wherein said first, second, and third glyceride copolymers are produced by a process comprising metathesis; in one aspect, said process comprises reacting two or more monomers in the presence of the metathesis catalyst as part of a reaction mixture, wherein the weight-to-weight ratio of the monomer compounds of formula (IIa) to the monomer compounds of formula (IIb) in the reaction mixture is no more than 10:1, no more than 9:1, no more than 8:1, no more than 7:1, no more than 6:1, no more than 5:1, no more than 4:1, no more than 3:1, no more than 2:1, or no more than 1:1; in one aspect, the metathesis catalyst is an organo-ruthenium compound, an organo-osmium compound, an organo-tungsten compound, or an organo-molybdenum compound.
   (d) The composition according to Paragraphs (a) through (c), wherein for said second glyceride copolymer at least one of R¹, R², R³, R⁴, or R⁵ is a C₉₋₁₃ alkenyl, in one aspect, at least one of R¹, R², R³, R⁴, or R⁵ is a C₉₋₁₂ alkenyl, in another aspect, at least one of R¹, R², R³, R⁴, or R⁵ is a C₉₋₁₀ alkenyl.
   (e) The composition according to Paragraphs (a) through (d), wherein for said third glyceride copolymer at least one of R¹¹, R¹², R¹³, R²¹, R²², or R²³ is a C₉₋₁₃ alkenyl, in one aspect, at least one of R¹¹, R¹², R¹³, R²¹, R²², or R²³ is a C₉₋₁₂ alkenyl, in another aspect, at least one of R¹¹, R¹², R¹³, R²¹, R²², or R²³ is a C₉₋₁₀ alkenyl.
   (f) The composition according to Paragraphs (a) through (e), wherein the second glyceride copolymer's G¹ and G² moieties are —CH₂— and G³ is a direct bond.
   (g) The composition according to any of Paragraphs (a) through (e), wherein the second glyceride copolymer's G¹ and G³ moieties are —CH₂— and G² is a direct bond.
   (h) The composition according to any of Paragraphs (a) through (e), wherein the second glyceride copolymer's G² and G³ moieties are —CH₂— and G¹ is a direct bond.
   (i) The composition according to Paragraphs (a) through (h), wherein for the second glyceride copolymer, at least one of, G⁴ and G⁵ are —CH₂— and G⁶ is a direct bond.
   (j) The composition according to any of Paragraphs (a) through (h), wherein for the second glyceride copolymer, at least one of, G⁴ and G⁶ are —CH₂— and G⁵ is a direct bond.
   (k) The composition according to any of Paragraphs (a) through (h), wherein for the second glyceride copolymer, at least one of, G⁵ and G⁶ are —CH₂— and G⁴ is a direct bond.
   (l) The composition according to any of Paragraphs (a) through (k), wherein for the second glyceride copolymer, at least one of, G⁷ and G⁸ are —CH₂— and G⁹ is a direct bond.
   (m) The composition according to Paragraphs (a) through (k), wherein for the second glyceride copolymer, at least one of G⁷ and G⁹ are —CH₂— and G⁸ is a direct bond.
   (n) The composition according to Paragraphs (a) through (k), wherein for the second glyceride copolymer, at least one of G⁸ and G⁹ are —CH₂— and G⁷ is a direct bond.
   (o) The composition according to any of Paragraphs (a) through (n), wherein for the second glyceride copolymer, each X¹ is independently selected from the group consisting of —(CH₂)₁₆—, —(CH₂)₁₈—, —(CH₂)₁₉—, —(CH₂)₂₀—, —(CH₂)₂₂—, —(CH₂)₂₄—, —(CH₂)₂₅—, —(CH₂)₂₈—, —(CH₂)₇—CH═CH—(CH₂)₇—, —(CH₂)₇—CH═CH—CH₂—CH═CH—(CH₂)₇—, —(CH₂)₇—CH═CH—CH₂—CH═CH—CH₂—CH═CH—(CH₂)₇—, —(CH₂)₇—CH═CH—CH₂—CH═CH—CH₂—CH═CH—CH₂—CH═CH—(CH₂)₇—, —(CH₂)₇—CH═CH—CH₂—CH═CH—CH₂—CH═CH—CH₂—CH═CH—CH₂—CH═CH—(CH₂)₇—, —(CH₂)₁₁—CH═CH—(CH₂)₁₁—, —(CH₂)₇—CH═CH—CH₂—CH═CH—(CH₂)₁₁—, —(CH₂)₁₁—CH═CH—CH₂—CH═CH—(CH₂)₇—, —(CH₂)₇—CH═CH—CH₂—CH═CH—CH₂—CH═CH—(CH₂)₁₁—, —(CH₂)₁₁—CH═CH—CH₂—CH═CH—CH₂—CH═CH—(CH₂)₇—, —(CH₂)₉—CH═CH—(CH₂)₇, —(CH₂)₇—CH═CH—(CH₂)₉, —(CH₂)₁₁—CH═CH—(CH₂)₇—, or —(CH₂)₇—CH═CH—(CH₂)₁₁—.
   (p) The composition according to any of Paragraphs (a) through (m), wherein for the second glyceride copolymer, each X² is independently selected from the group consisting of —(CH₂)₁₆—, —(CH₂)₁₈—, —(CH₂)₁₉—, —(CH₂)₂₀—, —(CH₂)₂₂—, —(CH₂)₂₄—, —(CH₂)₂₅—, —(CH₂)₂₈—, —(CH₂)₇—CH═CH—(CH₂)₇—, —(CH₂)₇—CH═CH—CH₂—CH═CH—(CH₂)₇—, —(CH₂)₇—CH═CH—CH₂—CH═CH—CH₂—CH═CH—(CH₂)₇—, —(CH₂)₇—CH═CH—CH₂—CH═CH—CH₂—CH═CH—CH₂—CH═CH—(CH₂)₇—, —(CH₂)₇—CH═CH—CH₂—CH═CH—CH₂—CH═CH—CH₂—CH═CH—CH₂—CH═CH—(CH₂)₇—, —(CH₂)₁₁—CH═CH—(CH₂)₁₁—, —(CH₂)₇—CH═CH—CH₂—CH═CH—(CH₂)₁₁—, —(CH₂)₁₁—CH═CH—CH₂—CH═CH—(CH₂)₇—, —(CH₂)₇—CH═CH—CH₂—CH═CH—CH₂—CH═CH—(CH₂)₁₁—, —(CH₂)₁₁—CH═CH—CH₂—CH═CH—CH₂—CH═CH—(CH₂)₇—, —(CH₂)₉—CH═CH—(CH₂)₇, —(CH₂)₇—CH═CH—(CH₂)₉, —(CH₂)₁₁—CH═CH—(CH₂)₇—, or —(CH₂)₇—CH═CH—(CH₂)₁₁—.
   (q) The composition according to any of Paragraphs (a) through (p), wherein for the second glyceride copolymer, R¹ is a C₁₋₂₄ alkyl or a C₂₋₂₄ alkenyl; in one aspect, R¹ is selected from the group consisting of: 8-nonenyl, 8-decenyl, 8-undecenyl, 8-dodecenyl, 8,11-dodecadienyl, 8,11-tridecadienyl, 8,11-tetradecadienyl, 8,11-pentadecadienyl, 8,11,14-pentadecatrienyl, 8,11,14-hexadecatrienyl, 8,11,14-octadecatrienyl, 9-methyl-8-decenyl, 9-methyl-8-undecenyl, 10-methyl-8-undecenyl, 12-methyl-8,11-tridecadienyl, 12-methyl-8,11-tetradecadienyl, 13-methyl-8,11-tetradecadienyl, 15-methyl-8,11,14-hexadecatrienyl, 15-methyl-8,11,14-heptadecatrienyl, 16-methyl-8,11,14-heptadecatrienyl, 12-tridecenyl, 12-tetradecenyl, 12-pentadecenyl, 12-hexadecenyl, 13-methyl-12-tetradecenyl, 13-methyl-12-pentadecenyl, and 14-methyl-12-pentadecenyl, in another aspect, R¹ is selected from the group consisting of 8-nonenyl, 8-decenyl, 8-undecenyl, 8,11-dodecadienyl, 8,11-tridecadienyl, 8,11-tetradecadienyl, 8,11,14-pentadecatrienyl, 8,11,14-hexadecatrienyl, 12-tridecenyl, 12-tetradecenyl, and 12-pentadecenyl.
   (r) The composition according to any of Paragraphs (a) through (q), wherein for the second glyceride copolymer, R² is a C₁₋₂₄ alkyl or a C₂₋₂₄ alkenyl; in one aspect, R² is selected from the group consisting of: 8-nonenyl, 8-decenyl, 8-undecenyl, 8-dodecenyl, 8,11-dodecadienyl, 8,11-tridecadienyl, 8,11-tetradecadienyl, 8,11-pentadecadienyl, 8,11,14-pentadecatrienyl, 8,11,14-hexadecatrienyl, 8,11,14-octadecatrienyl, 9-methyl-8-decenyl, 9-methyl-8-undecenyl, 10-methyl-8-undecenyl, 12-methyl-8,11-tridecadienyl, 12-methyl-8,11-tetradecadienyl, 13-methyl-8,11-tetradecadienyl, 15-methyl-8,11,14-hexadecatrienyl, 15-methyl-8,11,14-heptadecatrienyl, 16-methyl-8,11,14-heptadecatrienyl, 12-tridecenyl, 12-tetradecenyl, 12-pentadecenyl, 12-hexadecenyl, 13-methyl-12-tetradecenyl, 13-methyl-12-pentadecenyl, and 14-methyl-12-pentadecenyl; in another aspect, R² is selected from the group consisting of 8-nonenyl, 8-decenyl, 8-undecenyl, 8,11-dodecadienyl, 8,11-tridecadienyl, 8,11-tetradecadienyl, 8,11,14-pentadecatrienyl, 8,11,14-hexadecatrienyl, 12-tridecenyl, 12-tetradecenyl, and 12-pentadecenyl.
   (s) The composition according to any of Paragraphs (a) through (r), wherein for the second glyceride copolymer, R³ is a C₁₋₂₄ alkyl or a C₂₋₂₄ alkenyl; in one aspect, R³ is selected from the group consisting of: 8-nonenyl, 8-decenyl, 8-undecenyl, 8-dodecenyl, 8,11-dodecadienyl, 8,11-tridecadienyl, 8,11-tetradecadienyl, 8,11-pentadecadienyl, 8,11,14-pentadecatrienyl, 8,11,14-hexadecatrienyl, 8,11,14-octadecatrienyl, 9-methyl-8-decenyl, 9-methyl-8-undecenyl, 10-methyl-8-undecenyl, 12-methyl-8,11-tridecadienyl, 12-methyl-8,11-tetradecadienyl, 13-methyl-8,11-tetradecadienyl, 15-methyl-8,11,14-hexadecatrienyl, 15-methyl-8,11,14-heptadecatrienyl, 16-methyl-8,11,14-heptadecatrienyl, 12-tridecenyl, 12-tetradecenyl, 12-pentadecenyl, 12-hexadecenyl, 13-methyl-12-tetradecenyl, 13-methyl-12-pentadecenyl, and 14-methyl-12-pentadecenyl; in another aspect, R³ is selected from the group consisting of 8-nonenyl, 8-decenyl, 8-undecenyl, 8,11-dodecadienyl, 8,11-tridecadienyl, 8,11-tetradecadienyl, 8,11,14-pentadecatrienyl, 8,11,14-hexadecatrienyl, 12-tridecenyl, 12-tetradecenyl, and 12-pentadecenyl.
   (t) The composition according to any of Paragraphs (a) through (s), wherein for the second glyceride copolymer, each R⁴ is independently selected from a C₁₋₂₄ alkyl and a C₂₋₂₄ alkenyl; in one aspect, each R⁴ is independently selected from the group consisting of: 8-nonenyl, 8-decenyl, 8-undecenyl, 8-dodecenyl, 8,11-dodecadienyl, 8,11-tridecadienyl, 8,11-tetradecadienyl, 8,11-pentadecadienyl, 8,11,14-pentadecatrienyl, 8,11,14-hexadecatrienyl, 8,11,14-octadecatrienyl, 9-methyl-8-decenyl, 9-methyl-8-undecenyl, 10-methyl-8-undecenyl, 12-methyl-8,11-tridecadienyl, 12-methyl-8,11-tetradecadienyl, 13-methyl-8,11-tetradecadienyl, 15-methyl-8,11,14-hexadecatrienyl, 15-methyl-8,11,14-heptadecatrienyl, 16-methyl-8,11,14-heptadecatrienyl, 12-tridecenyl, 12-tetradecenyl, 12-pentadecenyl, 12-hexadecenyl, 13-methyl-12-tetradecenyl, 13-methyl-12-pentadecenyl, and 14-methyl-12-pentadecenyl; in another aspect, each R⁴ is independently selected from the group consisting of 8-nonenyl, 8-decenyl, 8-undecenyl, 8,11-dodecadienyl, 8,11-tridecadienyl, 8,11-tetradecadienyl, 8,11,14-pentadecatrienyl, 8,11,14-hexadecatrienyl, 12-tridecenyl, 12-tetradecenyl, and 12-pentadecenyl.
   (u) The composition according to any of Paragraphs (a) through (t), wherein for the second glyceride copolymer, R⁵ is a C₁₋₂₄ alkyl or a C₂₋₂₄ alkenyl; in one aspect, R⁵ is selected from the group consisting of: 8-nonenyl, 8-decenyl, 8-undecenyl, 8-dodecenyl, 8,11-dodecadienyl, 8,11-tridecadienyl, 8,11-tetradecadienyl, 8,11-pentadecadienyl, 8,11,14-pentadecatrienyl, 8,11,14-hexadecatrienyl, 8,11,14-octadecatrienyl, 9-methyl-8-decenyl, 9-methyl-8-undecenyl, 10-methyl-8-undecenyl, 12-methyl-8,11-tridecadienyl, 12-methyl-8,11-tetradecadienyl, 13-methyl-8,11-tetradecadienyl, 15-methyl-8,11,14-hexadecatrienyl, 15-methyl-8,11,14-heptadecatrienyl, 16-methyl-8,11,14-heptadecatrienyl, 12-tridecenyl, 12-tetradecenyl, 12-pentadecenyl, 12-hexadecenyl, 13-methyl-12-tetradecenyl, 13-methyl-12-pentadecenyl, and 14-methyl-12-pentadecenyl; in another aspect, R⁵ is selected from the group consisting of 8-nonenyl, 8-decenyl, 8-undecenyl, 8,11-dodecadienyl, 8,11-tridecadienyl, 8,11-tetradecadienyl, 8,11,14-pentadecatrienyl, 8,11,14-hexadecatrienyl, 12-tridecenyl, 12-tetradecenyl, and 12-pentadecenyl.
   (v) The composition according to any of Paragraphs (a) through (u), wherein for the second glyceride copolymer, n is an integer from 3 to 250, from 5 to 180, from 6 to 140, from 8 to 70, from 9 to 40, or from 9 to 26.
   (w) The composition according to Paragraphs (a) through (c), wherein for the third glyceride copolymer, R¹¹, R¹², and R¹³ are each independently selected from the group consisting of pentadecyl, heptadecyl, 8-heptadecenyl, 8,11-heptadecadienyl, and 8,11,14-heptadecatrienyl.
   (x) The composition according to Paragraphs (a) through (c) and (w), wherein for the third glyceride copolymer, two of R²¹, R²², and R²³ are independently selected from the group consisting of pentadecyl, heptadecyl, 8-heptadecenyl, 8,11-heptadecadienyl, and 8,11,14-heptadecatrienyl; and wherein one of R²¹, R²², and R²³ is selected from the group consisting of: 8-nonenyl, 8-decenyl, 8-undecenyl, 8-dodecenyl, 8,11-dodecadienyl, 8,11-tridecadienyl, 8,11-tetradecadienyl, 8,11-pentadecadienyl, 8,11,14-pentadecatrienyl, 8,11,14-hexadecatrienyl, 8,11,14-octadecatrienyl, 9-methyl-8-decenyl, 9-methyl-8-undecenyl, 10-methyl-8-undecenyl, 12-methyl-8,11-tridecadienyl, 12-methyl-8,11-tetradecadienyl, 13-methyl-8,11-tetradecadienyl, 15-methyl-8,11,14-hexadecatrienyl, 15-methyl-8,11,14-heptadecatrienyl, 16-methyl-8,11,14-heptadecatrienyl, 12-tridecenyl, 12-tetradecenyl, 12-pentadecenyl, 12-hexadecenyl, 13-methyl-12-tetradecenyl, 13-methyl-12-pentadecenyl, and 14-methyl-12-pentadecenyl; in one aspect, one of R²¹, R²², and R²³ is selected from the group consisting of 8-nonenyl, 8-decenyl, 8-undecenyl, 8,11-dodecadienyl, 8,11-tridecadienyl, 8,11-tetradecadienyl, 8,11,14-pentadecatrienyl, 8,11,14-hexadecatrienyl, 12-tridecenyl, 12-tetradecenyl, and 12-pentadecenyl.
   (y) The composition according to Paragraphs (a) through (c) and (w), wherein for the third glyceride copolymer, one of R²¹, R²², and R²³ is selected from the group consisting of pentadecyl, heptadecyl, 8-heptadecenyl, 8,11-heptadecadienyl, and 8,11,14-heptadecatrienyl; and wherein two of R²¹, R²², and R²³ are independently selected from the group consisting of: 8-nonenyl, 8-decenyl, 8-undecenyl, 8-dodecenyl, 8,11-dodecadienyl, 8,11-tridecadienyl, 8,11-tetradecadienyl, 8,11-pentadecadienyl, 8,11,14-pentadecatrienyl, 8,11,14-hexadecatrienyl, 8,11,14-octadecatrienyl, 9-methyl-8-decenyl, 9-methyl-8-undecenyl, 10-methyl-8-undecenyl, 12-methyl-8,11-tridecadienyl, 12-methyl-8,11-tetradecadienyl, 13-methyl-8,11-tetradecadienyl, 15-methyl-8,11,14-hexadecatrienyl, 15-methyl-8,11,14-heptadecatrienyl, 16-methyl-8,11,14-heptadecatrienyl, 12-tridecenyl, 12-tetradecenyl, 12-pentadecenyl, 12-hexadecenyl, 13-methyl-12-tetradecenyl, 13-methyl-12-pentadecenyl, and 14-methyl-12-pentadecenyl; in one aspect, two of R²¹, R²², and R²³ are independently selected from the group consisting of 8-nonenyl, 8-decenyl, 8-undecenyl, 8,11-dodecadienyl, 8,11-tridecadienyl, 8,11-tetradecadienyl, 8,11,14-pentadecatrienyl, 8,11,14-hexadecatrienyl, 12-tridecenyl, 12-tetradecenyl, and 12-pentadecenyl.
   (z) A composition comprising a glyceride copolymer, which comprises constitutional units formed from reacting:

a) at least an unsaturated natural oil glyceride, and an unsaturated alkenylized natural oil glyceride in the presence of a metathesis catalyst;

b) at least an unsaturated synthetic polyol ester, and an unsaturated alkenylized natural oil glyceride in the presence of a metathesis catalyst;

c) at least an unsaturated natural oil glyceride, and an unsaturated alkenylized synthetic polyol ester in the presence of a metathesis catalyst;

d) at least an unsaturated synthetic polyol ester, and an unsaturated alkenylized synthetic polyol ester in the presence of a metathesis catalyst;

e) at least an unsaturated alkenylized synthetic polyol ester, and an unsaturated alkenylized synthetic polyol ester in the presence of a metathesis catalyst;

f) at least an unsaturated alkenylized natural oil glyceride, and an unsaturated alkenylized natural oil glyceride in the presence of a metathesis catalyst;

in one aspect, said glyceride copolymer comprises a C₁₀₋₁₄ unsaturated fatty acid ester,
in one aspect said catalyst is selected from the group consisting of an organo-ruthenium compound, an organo-osmium compound, an organo-tungsten compound, an organo-molybdenum compound and mixtures thereof;
in one aspect the unsaturated alkenylized natural oil glyceride is formed from the reaction of an unsaturated natural oil glyceride with a short-chain alkene in the presence of a metathesis catalyst, in one aspect, said catalyst is selected from the group consisting of an organo-ruthenium compound, an organo-osmium compound, an organo-tungsten compound, an organo-molybdenum compound and mixtures thereof, in one aspect, the short-chain alkene is selected from the group consisting of ethylene, propylene, 1-butene, 2-butene, isobutene, 1-pentene, 2-pentene, 1-hexene, 2-hexene, 3-hexene and mixtures thereof, in one aspect, the short-chain alkene is selected from the group consisting of ethylene, propylene, 1-butene, and 2-butene, and mixtures thereof, in one aspect, the unsaturated alkenylized natural oil glyceride has a lower molecular weight than the second unsaturated natural oil glyceride;
in one aspect, the unsaturated natural oil glyceride is obtained from a natural oil; in one aspect, from vegetable oil, animal fat, and/or algae oil; in one aspect, from Abyssinian oil, Almond Oil, Apricot Oil, Apricot Kernel oil, Argan oil, Avocado Oil, Babassu Oil, Baobab Oil, Black Cumin Oil, Black Currant Oil, Borage Oil, Camelina oil, Carinata oil, Canola oil, Castor oil, Cherry Kernel Oil, Coconut oil, Corn oil, Cottonseed oil, Echium Oil, Evening Primrose Oil, Flax Seed Oil, Grape Seed Oil, Grapefruit Seed Oil, Hazelnut Oil, Hemp Seed Oil, Jatropha oil, Jojoba Oil, Kukui Nut Oil, Linseed Oil, Macadamia Nut Oil, Meadowfoam Seed Oil, Moringa Oil, Neem Oil, Olive Oil, Palm Oil, Palm Kernel Oil, Peach Kernel Oil, Peanut Oil, Pecan Oil, Pennycress oil, Perilla Seed Oil, Pistachio Oil, Pomegranate Seed Oil, Pongamia oil, Pumpkin Seed Oil, Raspberry Oil, Red Palm Olein, Rice Bran Oil, Rosehip Oil, Safflower Oil, Seabuckthorn Fruit Oil, Sesame Seed Oil, Shea Olein, Sunflower Oil, Soybean Oil, Tonka Bean Oil, Tung Oil, Walnut Oil, Wheat Germ Oil, High Oleoyl Soybean Oil, High Oleoyl Sunflower Oil, High Oleoyl Safflower Oil, High Erucic Acid Rapeseed Oil, and mixtures thereof;
in one aspect, said synthetic polyol ester is derived from a material selected from the group consisting of ethylene glycol, propylene glycol, glycerol, polyglycerol, polyethylene glycol, polypropylene glycol, poly(tetramethylene ether) glycol, pentaerythritol, dipentaerythritol, tripentaerythritol, trimethylolpropane, neopentyl glycol, a sugar, for example, sucrose, and mixtures thereof
in one aspect, the glyceride copolymer has a weight average molecular weight ranging from 4,000 g/mol to 150,000 g/mol, from 5,000 g/mol to 130,000 g/mol, from 6,000 g/mol to 100,000 g/mol, from 7,000 g/mol to 50,000 g/mol, from 8,000 g/mol to 30,000 g/mol, or from 8,000 g/mol to 20,000 g/mol.
(aa) The composition of Paragraph (z), wherein the short-chain alkene is ethylene
(bb) The composition of Paragraph (z), wherein the short-chain alkene is propylene.
(cc) The composition of Paragraph (z), wherein the short-chain alkene is 1-butene.
(dd) The composition of Paragraph (z), wherein the short-chain alkene is 2-butene.
(ee) A composition according to Paragraphs (a) through (c) wherein the first glyceride copolymer is derived from a natural polyol ester and/or a synthetic polyol ester, in one aspect, said natural polyol ester is selected from the group consisting of a vegetable oil, an animal fat, an algae oil and mixtures thereof; and said synthetic polyol ester is derived from a material selected from the group consisting of ethylene glycol, propylene glycol, glycerol, polyglycerol, polyethylene glycol, polypropylene glycol, poly(tetramethylene ether) glycol, pentaerythritol, dipentaerythritol, tripentaerythritol, trimethylolpropane, neopentyl glycol, a sugar, for example, sucrose, and mixtures thereof.
(ff) A composition according to any of Paragraphs (a) through (ee), said composition comprising, based on total composition weight, from about 0.1% to about 50%, from about 0.5% to about 30%, or from about 1% to about 20% of a glyceride copolymer, selected from the group consisting of said first glyceride copolymer, second glyceride copolymer, third glyceride copolymer, and mixtures thereof.
(gg) A composition according to any of Paragraphs (a) through (ff), wherein said first, and second, glyceride copolymers have a free hydrocarbon content, based on the weight of glyceride copolymer of from about 0% to about 5%, from about 0.1% to about 5%, from about 0.1% to about 4%, from about 0.1 to about 3%, or from about 0.1% to about 1%.
(hh) A composition according to any of Paragraphs (a) through (ii), wherein said third glyceride copolymer have a free hydrocarbon content, based on the weight of glyceride copolymer of from about 0% to about 5%, from about 0.1% to about 5%, from about 0.1% to about 4%, from about 0.1 to about 3%, or from about 0.1% to about 1%.
(ii) The composition according to any of Paragraphs (a) through (c) and (w), wherein for the third glyceride copolymer, R²¹, R²², and R²³ are each independently selected from the group consisting of: 8-nonenyl, 8-decenyl, 8-undecenyl, 8-dodecenyl, 8,11-dodecadienyl, 8,11-tridecadienyl, 8,11-tetradecadienyl, 8,11-pentadecadienyl, 8,11,14-pentadecatrienyl, 8,11,14-hexadecatrienyl, 8,11,14-octadecatrienyl, 9-methyl-8-decenyl, 9-methyl-8-undecenyl, 10-methyl-8-undecenyl, 12-methyl-8,11-tridecadienyl, 12-methyl-8,11-tetradecadienyl, 13-methyl-8,11-tetradecadienyl, 15-methyl-8,11,14-hexadecatrienyl, 15-methyl-8,11,14-heptadecatrienyl, 16-methyl-8,11,14-heptadecatrienyl, 12-tridecenyl, 12-tetradecenyl, 12-pentadecenyl, 12-hexadecenyl, 13-methyl-12-tetradecenyl, 13-methyl-12-pentadecenyl, and 14-methyl-12-pentadecenyl; in one aspect, R²¹, R²², and R²³ are each independently selected from the group consisting of 8-nonenyl, 8-decenyl, 8-undecenyl, 8,11-dodecadienyl, 8,11-tridecadienyl, 8,11-tetradecadienyl, 8,11,14-pentadecatrienyl, 8,11,14-hexadecatrienyl, 12-tridecenyl, 12-tetradecenyl, and 12-pentadecenyl.

### Methods of Making Compositions

The compositions of the present invention can be formulated into any suitable form and prepared by any process chosen by the formulator, non-limiting examples of which are described in U.S. Pat. No. 5,879,584 and U.S. patent application Ser. No. 12/491,478, which are incorporated herein by reference. For example, the glyceride copolymers can be combined directly with the composition's other ingredients without pre-emulsification and/or pre-mixing to form the finished products. Alternatively, the glyceride copolymers can be combined with surfactants or emulsifiers, solvents, suitable adjuncts, and/or any other suitable ingredients to prepare emulsions prior to compounding the finished products. In some embodiments, the glyceride copolymers can be added to the composition separately from the gel matrix. In such embodiments, where there is a discrete phase comprising the glyceride copolymers, the discrete phase can optionally have an average particle size in the hair care composition of from about 0.5 μm to about 20 μm. In other embodiments, the glyceride copolymers can be added to the gel matrix first and then this gel matrix is combined with other components of the composition.

Suitable equipment for use in the processes disclosed herein may include continuous stirred tank reactors, homogenizers, turbine agitators, recirculating pumps, paddle mixers, plough shear mixers, ribbon blenders, vertical axis granulators and drum mixers, both in batch and, where available, in continuous process configurations, spray dryers, and extruders. Such equipment can be obtained from Lodige GmbH (Paderborn, Germany), Littleford Day, Inc. (Florence, Ky., U.S.A.), Forberg AS (Larvik, Norway), Glatt Ingenieurtechnik GmbH (Weimar, Germany), Niro (Soeborg, Denmark), Hosokawa Bepex Corp. (Minneapolis, Minn., U.S.A.), Arde Barinco (New Jersey, U.S.A.).

### A. Glyceride Oligomers

The hair care composition comprises, based on total composition weight, from about 0.05% to about 30%, from about 0.1% to about 15%, from about 0.25% to about 10%, or from about 0.5% to about 5%, of the glyceride oligomers described herein.

In one aspect, the disclosure provides glyceride copolymers of formula (I):

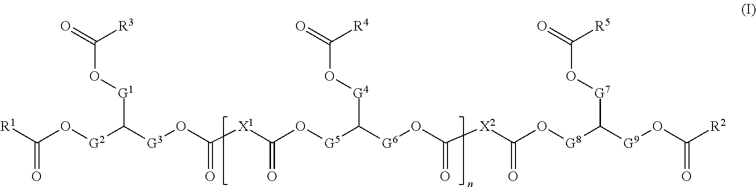

wherein: each R¹, R², R³, R⁴, and R⁵ is independently selected from the group consisting of an oligomeric glyceride moiety, a C₁₋₂₄ alkyl, a substituted C₁₋₂₄ alkyl wherein the substituent is one or more —OH moieties, a C₂₋₂₄ alkenyl, or a substituted C₂₋₂₄ alkenyl wherein the substituent is one or more —OH moieties; and/or each of the following combinations of moieties may each independently be covalently linked: R¹ and R³, R² and R⁵, R¹ and an adjacent R⁴, R² and an adjacent R⁴, R³ and an adjacent R⁴, R⁵ and an adjacent R⁴, or any two adjacent R⁴ such that the covalently linked moieties forms an alkenylene moiety; each X¹ and X² is independently selected from the group consisting of a C₁₋₃₂ alkylene, a substituted C₁₋₃₂ alkylene wherein the substituent is one or more —OH moieties, a C₂₋₃₂ alkenylene or a substituted C₂₋₃₂ alkenylene wherein the substituent is one or more —OH moieties; two of G¹, G², and G³ are —CH₂—, and one of G¹, G², and G³ is a direct bond; for each individual repeat unit in the repeat unit having index n, two of G⁴, G⁵, and G⁶ are —CH₂—, and one of G⁴, G⁵, and G⁶ is a direct bond, and the values G⁴, G⁵, and G⁶ for each individual repeat unit are independently selected from the values of G⁴, G⁵, and G⁶ in other repeating units; two of G⁷, G⁸, and G⁹ are —CH₂—, and one of G⁷, G⁸, and G⁹ is a direct bond; and n is an integer from 3 to 250; with the proviso for each of said second glyceride copolymers at least one of R¹, R², R³, and R⁵, and/or at least one R⁴ in one individual repeat unit of said repeat unit having index n, is selected from the group consisting of: 8-nonenyl; 8-decenyl; 8-undecenyl; 8-dodecenyl; 8,11-dodecadienyl; 8,11-tridecadienyl; 8,11-tetradecadienyl; 8,11-pentadecadienyl; 8,11,14-pentadecatrienyl; 8,11,14-hexadecatrienyl; 8,11,14-octadecatrienyl; 9-methyl-8-decenyl; 9-methyl-8-undecenyl; 10-methyl-8-undecenyl; 12-methyl-8,11-tridecadienyl; 12-methyl-8,11-tetradecadienyl; 13-methyl-8,11-tetradecadienyl; 15-methyl-8,11,14-hexadecatrienyl; 15-methyl-8,11,14-heptadecatrienyl; 16-methyl-8,11,14-heptadecatrienyl; 12-tridecenyl; 12-tetradecenyl; 12-pentadecenyl; 12-hexadecenyl; 13-methyl-12-tetradecenyl; 13-methyl-12-pentadecenyl; and 14-methyl-12-pentadecenyl.

G¹, G², and G³ can have any suitable value. In some embodiments, G¹ and G² are —CH₂— and G³ is a direct bond. In some other embodiments, G¹ and G³ are —CH₂— and G² is a direct bond. In some other embodiments, G² and G³ are —CH₂— and G¹ is a direct bond.

G⁴, G⁵, and G⁶ can, in each instance, independently have any suitable value. In some embodiments of any of the aforementioned embodiments, in at least one instance, G⁴ and G⁵ are —CH₂— and G⁶ is a direct bond. In some other embodiments of any of the aforementioned embodiments, in at least one instance, G⁴ and G⁶ are —CH₂— and G⁵ is a direct bond. In some other embodiments of any of the aforementioned embodiments, in at least one instance, G⁵ and G⁶ are —CH₂— and G⁴ is a direct bond.

G⁷, G⁸, and G⁹ can have any suitable value. In some embodiments of any of the aforementioned embodiments, G⁷ and G⁸ are —CH₂— and G⁹ is a direct bond. In some other embodiments of any of the aforementioned embodiments, G⁷ and G⁹ are —CH₂— and G⁸ is a direct bond. In some other embodiments of any of the aforementioned embodiments, G⁸ and G⁹ are —CH₂— and G⁷ is a direct bond.

X¹ can have any suitable value. In some embodiments of any of the aforementioned embodiments, X¹ is —(CH₂)₁₆—, —(CH₂)₁₈—, —(CH₂)₁₉—, —(CH₂)₂₀—, —(CH₂)₂₂—, —(CH₂)₂₄—, —(CH₂)₂₅—, —(CH₂)₂₈—, —(CH₂)₇—CH═CH—(CH₂)₇—, —(CH₂)₇—CH═CH—CH₂—CH═CH—(CH₂)₇—, —(CH₂)₇—CH═CH—CH₂—CH═CH—CH₂—CH═CH—(CH₂)₇—, —(CH₂)₇—CH═CH—CH₂—CH═CH—CH₂—CH═CH—CH₂—CH═CH—(CH₂)₇—, —(CH₂)₇—CH═CH—CH₂—CH═CH—CH₂—CH═CH—CH₂—CH═CH—CH₂—CH═CH—(CH₂)₇—, —(CH₂)₁₁—CH═CH—(CH₂)₁₁—, —(CH₂)₇—CH═CH—CH₂—CH═CH—(CH₂)₁₁—, —(CH₂)₁₁—CH═CH—CH₂—CH═CH—(CH₂)₇—, —(CH₂)₇—CH═CH—CH₂—CH═CH—CH₂—CH═CH—(CH₂)₁₁—, —(CH₂)₁₁—CH═CH—CH₂—CH═CH—CH₂—CH═CH—(CH₂)₇—, —(CH₂)₉—CH═CH—(CH₂)₇, —(CH₂)₇—CH═CH—(CH₂)₉, —(CH₂)₁₁—CH═CH—(CH₂)₇—, or —(CH₂)₇—CH═CH—(CH₂)₁₁—. In some such embodiments, X¹ is —(CH₂)₁₆—, —(CH₂)₁₈—, —(CH₂)₁₉—, —(CH₂)₂₂—, —(CH₂)₂₅—, —(CH₂)₂₈—, —(CH₂)₇—CH═CH—(CH₂)₇—, —(CH₂)₉—CH═CH—(CH₂)₇—, —(CH₂)₇—CH═CH—(CH₂)₉—, —(CH₂)₇—CH═CH—CH₂—CH═CH—(CH₂)₇—, —(CH₂)₇—CH═CH—CH₂—CH═CH—CH₂—CH═CH—(CH₂)₇—, —(CH₂)₇—CH═CH—CH₂—CH═CH—CH₂—CH═CH—CH₂—CH═CH—(CH₂)₇—, or —(CH₂)₇—CH═CH—CH₂—CH═CH—CH₂—CH═CH—CH₂—CH═CH—CH₂—CH═CH—(CH₂)₇—. In some such embodiments, X¹ is —(CH₂)₁₆—, —(CH₂)₁₉—, —(CH₂)₂₂—, —(CH₂)₂₅—, —(CH₂)₂₈—, —(CH₂)₇—CH═CH—(CH₂)₇—, —(CH₂)₇—CH═CH—CH₂—CH═CH—(CH₂)₇—, —(CH₂)₇—CH═CH—CH₂—CH═CH—CH₂—CH═CH—(CH₂)₇—, —(CH₂)₇—CH═CH—CH₂—CH═CH—CH₂—CH═CH—CH₂—CH═CH—(CH₂)₇—, or —(CH₂)₇—CH═CH—CH₂—CH═CH—CH₂—CH═CH—CH₂—CH═CH—CH₂—CH═CH—(CH₂)₇—. In some further such embodiments, X¹ is —(CH₂)₇—CH═CH—(CH₂)₇—, —(CH₂)₉—CH═CH—(CH₂)₇—, —(CH₂)₇—CH═CH—(CH₂)₉—, —(CH₂)₇—CH═CH—CH₂—CH═CH—(CH₂)₇—, —(CH₂)₇—CH═CH—CH₂—CH═CH—CH₂—CH═CH—(CH₂)₇—, —(CH₂)₇—CH═CH—CH₂—CH═CH—CH₂—CH═CH—CH₂—CH═CH—(CH₂)₇—, or —(CH₂)₇—CH═CH—CH₂—CH═CH—CH₂—CH═CH—CH₂—CH═CH—CH₂—CH═CH—(CH₂)₇—. In some further such embodiments, X¹ is —(CH₂)₇—CH═CH—(CH₂)₇—, —(CH₂)₇—CH═CH—CH₂—CH═CH—(CH₂)₇—, —(CH₂)₇—CH═CH—CH₂—CH═CH—CH₂—CH═CH—(CH₂)₇—, —(CH₂)₇—CH═CH—CH₂—CH═CH—CH₂—CH═CH—CH₂—CH═CH—(CH₂)₇—, or —(CH₂)₇—CH═CH—CH₂—CH═CH—CH₂—CH═CH—CH₂—CH═CH—CH₂—CH═CH—(CH₂)₇—.

X² can have any suitable value. In some embodiments of any of the aforementioned embodiments, X² is —(CH₂)₁₆—, —(CH₂)₁₈—, —(CH₂)₁₉—, —(CH₂)₂₀—, —(CH₂)₂₂—, —(CH₂)₂₄—, —(CH₂)₂₅—, —(CH₂)₂₈—, —(CH₂)₇—CH═CH—(CH₂)₇—, —(CH₂)₇—CH═CH—CH₂—CH═CH—(CH₂)₇—, —(CH₂)₇—CH═CH—CH₂—CH═CH—CH₂—CH═CH—(CH₂)₇—, —(CH₂)₇—CH═CH—CH₂—CH═CH—CH₂—CH═CH—CH₂—CH═CH—(CH₂)₇—, —(CH₂)₇—CH═CH—CH₂—CH═CH—CH₂—CH═CH—CH₂—CH═CH—CH₂—CH═CH—(CH₂)₇—, —(CH₂)₁₁—CH═CH—(CH₂)₁₁—, —(CH₂)₇—CH═CH—CH₂—CH═CH—(CH₂)₁₁—, —(CH₂)₁₁—CH═CH—CH₂—CH═CH—(CH₂)₇—, —(CH₂)₇—CH═CH—CH₂—CH═CH—CH₂—CH═CH—(CH₂)₁₁—, —(CH₂)₁₁—CH═CH—CH₂—CH═CH—CH₂—CH═CH—(CH₂)₇—, —(CH₂)₉—CH═CH—(CH₂)₇, —(CH₂)₇—CH═CH—(CH₂)₉, —(CH₂)₁₁—CH═CH—(CH₂)₇—, or —(CH₂)₇—CH═CH—(CH₂)₁₁—. In some such embodiments, X² is —(CH₂)₁₆—, —(CH₂)₁₈—, —(CH₂)₁₉—, —(CH₂)₂₂—, —(CH₂)₂₅—, —(CH₂)₂₈—, —(CH₂)₇—CH═CH—(CH₂)₇—, —(CH₂)₉—CH═CH—(CH₂)₇—, —(CH₂)₇—CH═CH—(CH₂)₉—, —(CH₂)₇—CH═CH—CH₂—CH═CH—(CH₂)₇—, —(CH₂)₇—CH═CH—CH₂—CH═CH—CH₂—CH═CH—(CH₂)₇—, —(CH₂)₇—CH═CH—CH₂—CH═CH—CH₂—CH═CH—CH₂—CH═CH—(CH₂)₇—, or —(CH₂)₇—CH═CH—CH₂—CH═CH—CH₂—CH═CH—CH₂—CH═CH—CH₂—CH═CH—(CH₂)₇—. In some such embodiments, X² is —(CH₂)₁₆—, —(CH₂)₁₉—, —(CH₂)₂₂—, —(CH₂)₂₅—, —(CH₂)₂₈—, —(CH₂)₇—CH═CH—(CH₂)₇—, —(CH₂)₇—CH═CH—CH₂—CH═CH—(CH₂)₇—, —(CH₂)₇—CH═CH—CH₂—CH═CH—CH₂—CH═CH—(CH₂)₇—, —(CH₂)₇—CH═CH—CH₂—CH═CH—CH₂—CH═CH—CH₂—CH═CH—(CH₂)₇—, or —(CH₂)₇—CH═CH—CH₂—CH═CH—CH₂—CH═CH—CH₂—CH═CH—CH₂—CH═CH—(CH₂)₇—. In some further such embodiments, X² is —(CH₂)₇—CH═CH—(CH₂)₇—, —(CH₂)₉—CH═CH—(CH₂)₇—, —(CH₂)₇—CH═CH—(CH₂)₉—, —(CH₂)₇—CH═CH—CH₂—CH═CH—(CH₂)₇—, —(CH₂)₇—CH═CH—CH₂—CH═CH—CH₂—CH═CH—(CH₂)₇—, —(CH₂)₇—CH═CH—CH₂—CH═CH—CH₂—CH═CH—CH₂—CH═CH—(CH₂)₇—, or —(CH₂)₇—CH═CH—CH₂—CH═CH—CH₂—CH═CH—CH₂—CH═CH—CH₂—CH═CH—(CH₂)₇—. In some further such embodiments, X² is —(CH₂)₇—CH═CH—(CH₂)₇—, —(CH₂)₇—CH═CH—CH₂—CH═CH—(CH₂)₇—, —(CH₂)₇—CH═CH—CH₂—CH═CH—CH₂—CH═CH—(CH₂)₇—, —(CH₂)₇—CH═CH—CH₂—CH═CH—CH₂—CH═CH—CH₂—CH═CH—(CH₂)₇—, or —(CH₂)₇—CH═CH—CH₂—CH═CH—CH₂—CH═CH—CH₂—CH═CH—CH₂—CH═CH—(CH₂)₇—.

R¹ can have any suitable value. In some embodiments of any of the aforementioned embodiments, R¹ is C₁₋₂₄ alkyl, or C₁₁₋₂₄ alkyl, or C₁₃₋₂₄ alkyl, or C₁₅₋₂₄ alkyl. In some such embodiments, R¹ is undecyl, tridecyl, pentadecyl, or heptadecyl. In some further such embodiments, R¹ is pentadecyl or heptadecyl. In some embodiments of any of the aforementioned embodiments, R¹ is C₂₋₂₄ alkenyl or C₉₋₂₄ alkenyl. In some such embodiments, R¹ is 8-heptadecenyl, 10-heptadecenyl, 12-heneicosenyl, 8,11-heptadecadienyl, 8,11,14-heptadecatrienyl, 8-nonenyl, 8-decenyl, 8-undecenyl, 10-undecenyl, 8-dodecenyl, 12-tridecenyl, 12-tetradecenyl, 12-pentadecenyl, 12-hexadecenyl, 9-methyl-8-decenyl, 9-methyl-8-undecenyl, 10-methyl-8-undecenyl, 13-methyl-12-tetradecenyl, 13-methyl-12-pentadecenyl, 14-methyl-12-pentadecenyl, 8,11-dodecadienyl, 8,11-tridecadienyl, 8,11-tetradecadienyl, 8,11-pentadecadienyl, 12-methyl-8,11-tridecadienyl, 12-methyl-8,11-tetradecadienyl, 13-methyl-8,11-tetradecadienyl, 15-methyl-8,11,14-hexadecatrienyl, 15-methyl-8,11,14-heptadecatrienyl, 16-methyl-8,11,14-heptadecatrienyl, 8,11,14-pentadecatrienyl, 8,11,14-hexadecatrienyl, 8,11,14-heptadecatrienyl, or 8,11,14-octadecatrienyl. In some further such embodiments, R¹ is 8-heptadecenyl, 10-heptadecenyl, 8,11-heptadecadienyl, or 8,11,14-heptadecatrienyl. In some further such embodiments, R¹ is 8-heptadecenyl, 8,11-heptadecadienyl, or 8,11,14-heptadecatrienyl. In some such embodiments, R¹ is 8-nonenyl, 8-decenyl, 8-undecenyl, 10-undecenyl, 8-dodecenyl, 8,11-dodecadienyl, 8,11-tridecadienyl, 12-tridecenyl, 8,11-tetradecadienyl, 8,11-pentadecadienyl, 8,11,14-pentadecatrienyl, 8,11,14-hexadecatrienyl, 8,11,14-heptadecatrienyl, or 8,11,14-octadecatrienyl. In some further such embodiments, R¹ is 8-nonenyl, 8-decenyl, 8-undecenyl, 8-dodecenyl, 8,11-dodecadienyl, 8,11-tridecadienyl, 8,11-tetradecadienyl, 8,11-pentadecadienyl, 8,11,14-pentadecatrienyl, 8,11,14-hexadecatrienyl, 8,11,14-heptadecatrienyl, or 8,11,14-octadecatrienyl. In some further such embodiments, R¹ is 8-nonenyl, 8-undecenyl, 8,11-dodecadienyl, 8,11-tetradecadienyl, or 8,11,14-pentadecatrienyl. In some embodiments, R¹ is an oligomeric glyceride moiety.

R² can have any suitable value. In some embodiments of any of the aforementioned embodiments, R² is C₁₋₂₄ alkyl, or C₁₁₋₂₄ alkyl, or C₁₃₋₂₄ alkyl, or C₁₅₋₂₄ alkyl. In some such embodiments, R² is undecyl, tridecyl, pentadecyl, or heptadecyl. In some further such embodiments, R² is pentadecyl or heptadecyl. In some embodiments of any of the aforementioned embodiments, R² is C₂₋₂₄ alkenyl or C₉₋₂₄ alkenyl In some such embodiments, R² is 8-heptadecenyl, 10-heptadecenyl, 12-heneicosenyl, 8,11-heptadecadienyl, 8,11,14-heptadecatrienyl, 8-nonenyl, 8-decenyl, 8-undecenyl, 10-undecenyl, 8-dodecenyl, 12-tridecenyl, 12-tetradecenyl, 12-pentadecenyl, 12-hexadecenyl, 9-methyl-8-decenyl, 9-methyl-8-undecenyl, 10-methyl-8-undecenyl, 13-methyl-12-tetradecenyl, 13-methyl-12-pentadecenyl, 14-methyl-12-pentadecenyl, 8,11-dodecadienyl, 8,11-tridecadienyl, 8,11-tetradecadienyl, 8,11-pentadecadienyl, 12-methyl-8,11-tridecadienyl, 12-methyl-8,11-tetradecadienyl, 13-methyl-8,11-tetradecadienyl, 15-methyl-8,11,14-hexadecatrienyl, 15-methyl-8,11,14-heptadecatrienyl, 16-methyl-8,11,14-heptadecatrienyl, 8,11,14-pentadecatrienyl, 8,11,14-hexadecatrienyl, 8,11,14-heptadecatrienyl, or 8,11,14-octadecatrienyl. In some further such embodiments, R² is 8-heptadecenyl, 10-heptadecenyl, 8,11-heptadecadienyl, or 8,11,14-heptadecatrienyl. In some further such embodiments, R² is 8-heptadecenyl, 8,11-heptadecadienyl, or 8,11,14-heptadecatrienyl. In some such embodiments, R² is 8-nonenyl, 8-decenyl, 8-undecenyl, 10-undecenyl, 8-dodecenyl, 8,11-dodecadienyl, 8,11-tridecadienyl, 8,11-tetradecadienyl, 8,11-pentadecadienyl, 8,11,14-pentadecatrienyl, 8,11,14-hexadecatrienyl, 8,11,14-heptadecatrienyl, or 8,11,14-octadecatrienyl. In some further such embodiments, R² is 8-nonenyl, 8-decenyl, 8-undecenyl, 8-dodecenyl, 8,11-dodecadienyl, 8,11-tridecadienyl, 12-tridecenyl, 8,11-tetradecadienyl, 8,11-pentadecadienyl, 8,11,14-pentadecatrienyl, 8,11,14-hexadecatrienyl, 8,11,14-heptadecatrienyl, or 8,11,14-octadecatrienyl. In some further such embodiments, R² is 8-nonenyl, 8-undecenyl, 8,11-dodecadienyl, 8,11-tetradecadienyl, or 8,11,14-pentadecatrienyl. In some embodiments, R² is an oligomeric glyceride moiety.

R³ can have any suitable value. In some embodiments of any of the aforementioned embodiments, R³ is C₁₋₂₄ alkyl, or C₁₁₋₂₄ alkyl, or C₁₃₋₂₄ alkyl, or C₁₅₋₂₄ alkyl. In some such embodiments, R³ is undecyl, tridecyl, pentadecyl, or heptadecyl. In some further such embodiments, R³ is pentadecyl or heptadecyl. In some embodiments of any of the aforementioned embodiments, R³ is C₂₋₂₄ alkenyl or C₉₋₂₄ alkenyl. In some such embodiments, R³ is 8-heptadecenyl, 10-heptadecenyl, 12-heneicosenyl, 8,11-heptadecadienyl, 8,11,14-heptadecatrienyl, 8-nonenyl, 8-decenyl, 8-undecenyl, 10-undecenyl, 8-dodecenyl, 12-tridecenyl, 12-tetradecenyl, 12-pentadecenyl, 12-hexadecenyl, 9-methyl-8-decenyl, 9-methyl-8-undecenyl, 10-methyl-8-undecenyl, 13-methyl-12-tetradecenyl, 13-methyl-12-pentadecenyl, 14-methyl-12-pentadecenyl, 8,11-dodecadienyl, 8,11-tridecadienyl, 8,11-tetradecadienyl, 8,11-pentadecadienyl, 12-methyl-8,11-tridecadienyl, 12-methyl-8,11-tetradecadienyl, 13-methyl-8,11-tetradecadienyl, 15-methyl-8,11,14-hexadecatrienyl, 15-methyl-8,11,14-heptadecatrienyl, 16-methyl-8,11,14-heptadecatrienyl, 8,11,14-pentadecatrienyl, 8,11,14-hexadecatrienyl, 8,11,14-heptadecatrienyl, or 8,11,14-octadecatrienyl. In some further such embodiments, R³ is 8-heptadecenyl, 10-heptadecenyl, 8,11-heptadecadienyl, or 8,11,14-heptadecatrienyl. In some further such embodiments, R³ is 8-heptadecenyl, 8,11-heptadecadienyl, or 8,11,14-heptadecatrienyl. In some such embodiments, R³ is 8-nonenyl, 8-decenyl, 8-undecenyl, 10-undecenyl, 8-dodecenyl, 8,11-dodecadienyl, 8,11-tridecadienyl, 8,11-tetradecadienyl, 8,11-pentadecadienyl, 8,11,14-pentadecatrienyl, 8,11,14-hexadecatrienyl, 8,11,14-heptadecatrienyl, or 8,11,14-octadecatrienyl. In some further such embodiments, R³ is 8-nonenyl, 8-decenyl, 8-undecenyl, 8-dodecenyl, 8,11-dodecadienyl, 8,11-tridecadienyl, 12-tridecenyl, 8,11-tetradecadienyl, 8,11-pentadecadienyl, 8,11,14-pentadecatrienyl, 8,11,14-hexadecatrienyl, 8,11,14-heptadecatrienyl, or 8,11,14-octadecatrienyl. In some further such embodiments, R³ is 8-nonenyl, 8-undecenyl, 8,11-dodecadienyl, 8,11-tetradecadienyl, or 8,11,14-pentadecatrienyl. In some embodiments, R³ is an oligomeric glyceride moiety.

R⁴ can, in each of its instances, have any suitable value. In some embodiments of any of the aforementioned embodiments, R⁴, in at least one instance, is C₁₋₂₄ alkyl, or C₁₁₋₂₄ alkyl, or C₁₃₋₂₄ alkyl, or C₁₅₋₂₄ alkyl. In some such embodiments, R⁴ is, in at least one instance, undecyl, tridecyl, pentadecyl, or heptadecyl. In some further such embodiments, R⁴ is, in at least one instance, pentadecyl or heptadecyl. In some embodiments of any of the aforementioned embodiments, R⁴ is, in at least one instance, C₂₋₂₄ alkenyl or C₉₋₂₄ alkenyl. In some such embodiments, R⁴ is, in at least one instance, 8-heptadecenyl, 10-heptadecenyl, 12-heneicosenyl, 8,11-heptadecadienyl, 8,11,14-heptadecatrienyl, 8-nonenyl, 8-decenyl, 8-undecenyl, 10-undecenyl, 8-dodecenyl, 12-tridecenyl, 12-tetradecenyl, 12-pentadecenyl, 12-hexadecenyl, 9-methyl-8-decenyl, 9-methyl-8-undecenyl, 10-methyl-8-undecenyl, 13-methyl-12-tetradecenyl, 13-methyl-12-pentadecenyl, 14-methyl-12-pentadecenyl, 8,11-dodecadienyl, 8,11-tridecadienyl, 8,11-tetradecadienyl, 8,11-pentadecadienyl, 12-methyl-8,11-tridecadienyl, 12-methyl-8,11-tetradecadienyl, 13-methyl-8,11-tetradecadienyl, 15-methyl-8,11,14-hexadecatrienyl, 15-methyl-8,11,14-heptadecatrienyl, 16-methyl-8,11,14-heptadecatrienyl, 8,11,14-pentadecatrienyl, 8,11,14-hexadecatrienyl, 8,11,14-heptadecatrienyl, or 8,11,14-octadecatrienyl. In some further such embodiments, R⁴ is, in at least one instance, 8-heptadecenyl, 10-heptadecenyl, 8,11-heptadecadienyl, or 8,11,14-heptadecatrienyl. In some further such embodiments, R⁴ is, in at least one instance, 8-heptadecenyl, 8,11-heptadecadienyl, or 8,11,14-heptadecatrienyl. In some such embodiments, R⁴ is, in at least one instance, 8-nonenyl, 8-decenyl, 8-undecenyl, 10-undecenyl, 8-dodecenyl, 8,11-dodecadienyl, 8,11-tridecadienyl, 12-tridecenyl, 8,11-tetradecadienyl, 8,11-pentadecadienyl, 8,11,14-pentadecatrienyl, 8,11,14-hexadecatrienyl, 8,11,14-heptadecatrienyl, or 8,11,14-octadecatrienyl. In some further such embodiments, R⁴ is, in at least one instance, 8-nonenyl, 8-decenyl, 8-undecenyl, 8-dodecenyl, 8,11-dodecadienyl, 8,11-tridecadienyl, 8,11-tetradecadienyl, 8,11-pentadecadienyl, 8,11,14-pentadecatrienyl, 8,11,14-hexadecatrienyl, 8,11,14-heptadecatrienyl, or 8,11,14-octadecatrienyl. In some further such embodiments, R⁴ is, in at least one instance, 8-nonenyl, 8-undecenyl, 8,11-dodecadienyl, 8,11-tetradecadienyl, or 8,11,14-pentadecatrienyl. In some embodiments, R⁴, in at least one instance, is an oligomeric glyceride moiety.

R⁵ can have any suitable value. In some embodiments of any of the aforementioned embodiments, R⁵ is C₁₋₂₄ alkyl, or C₁₁₋₂₄ alkyl, or C₁₃₋₂₄ alkyl, or C₁₅₋₂₄ alkyl. In some such embodiments, R⁵ is undecyl, tridecyl, pentadecyl, or heptadecyl. In some further such embodiments, R⁵ is pentadecyl or heptadecyl. In some embodiments of any of the aforementioned embodiments, R⁵ is C₂₋₂₄ alkenyl or C₉₋₂₄ alkenyl. In some such embodiments, R⁵ is 8-heptadecenyl, 10-heptadecenyl, 12-heneicosenyl, 8,11-heptadecadienyl, 8,11,14-heptadecatrienyl, 8-nonenyl, 8-decenyl, 8-undecenyl, 10-undecenyl, 8-dodecenyl, 12-tridecenyl, 12-tetradecenyl, 12-pentadecenyl, 12-hexadecenyl, 9-methyl-8-decenyl, 9-methyl-8-undecenyl, 10-methyl-8-undecenyl, 13-methyl-12-tetradecenyl, 13-methyl-12-pentadecenyl, 14-methyl-12-pentadecenyl, 8,11-dodecadienyl, 8,11-tridecadienyl, 8,11-tetradecadienyl, 8,11-pentadecadienyl, 12-methyl-8,11-tridecadienyl, 12-methyl-8,11-tetradecadienyl, 13-methyl-8,11-tetradecadienyl, 15-methyl-8,11,14-hexadecatrienyl, 15-methyl-8,11,14-heptadecatrienyl, 16-methyl-8,11,14-heptadecatrienyl, 8,11,14-pentadecatrienyl, 8,11,14-hexadecatrienyl, 8,11,14-heptadecatrienyl, or 8,11,14-octadecatrienyl. In some further such embodiments, R⁵ is 8-heptadecenyl, 10-heptadecenyl, 8,11-heptadecadienyl, or 8,11,14-heptadecatrienyl. In some further such embodiments, R⁵ is 8-heptadecenyl, 8,11-heptadecadienyl, or 8,11,14-heptadecatrienyl. In some such embodiments, R⁵ is 8-nonenyl, 8-decenyl, 8-undecenyl, 10-undecenyl, 8-dodecenyl, 8,11-dodecadienyl, 12-tridecenyl, 8,11-tridecadienyl, 8,11-tetradecadienyl, 8,11-pentadecadienyl, 8,11,14-pentadecatrienyl, 8,11,14-hexadecatrienyl, 8,11,14-heptadecatrienyl, or 8,11,14-octadecatrienyl. In some further such embodiments, R⁵ is 8-nonenyl, 8-decenyl, 8-undecenyl, 8-dodecenyl, 8,11-dodecadienyl, 8,11-tridecadienyl, 8,11-tetradecadienyl, 8,11-pentadecadienyl, 8,11,14-pentadecatrienyl, 8,11,14-hexadecatrienyl, 8,11,14-heptadecatrienyl, or 8,11,14-octadecatrienyl. In some further such embodiments, R⁵ is 8-nonenyl, 8-undecenyl, 8,11-dodecadienyl, 8,11-tetradecadienyl, or 8,11,14-pentadecatrienyl. In some embodiments, R⁵ is an oligomeric glyceride moiety.

The variable n can have any suitable value. In some embodiments of any of the aforementioned embodiments, n is an integer from 3 to 250, or from 5 to 180, or from 6 to 140, or from 8 to 70, or from 9 to 40, or from 9 to 26. In some other embodiments, n is an integer from 3 to 35, or from 5 to 30, or from 7 to 25, or from 10 to 20.

In some embodiments of any of the aforementioned embodiments, the glyceride polymers include only compounds wherein at least one of R¹, R², R³, and R⁵, or at least one instance of R⁴, is selected from the group consisting of: 8-nonenyl; 8-decenyl; 8-undecenyl; 10-undecenyl, 12-tridecenyl; 8-dodecenyl; 8,11-dodecadienyl; 8,11-tridecadienyl; 8,11-tetradecadienyl; 8,11-pentadecadienyl; 8,11,14-pentadecatrienyl; 8,11,14-hexadecatrienyl; 8,11,14-heptadecatrienyl; and 8,11,14-octadecatrienyl. In some other embodiments of any of the aforementioned embodiments, the glyceride polymers include only compounds wherein at least one of R¹, R², R³, and R⁵, or at least one instance of R⁴, is selected from the group consisting of: 8-nonenyl; 8-decenyl; 8-undecenyl; 8-dodecenyl; 8,11-dodecadienyl; 8,11-tridecadienyl; 8,11-tetradecadienyl; 8,11-pentadecadienyl; 8,11,14-pentadecatrienyl; 8,11,14-hexadecatrienyl; 8,11,14-heptadecatrienyl; and 8,11,14-octadecatrienyl. In some other embodiments of any of the aforementioned embodiments, the glyceride polymers include only compounds wherein at least one of R¹, R², R³, and R⁵, or at least one instance of R⁴, is selected from the group consisting of: 8-nonenyl; 8-undecenyl; 8,11-dodecadienyl; 8,11-tetradecadienyl; or 8,11,14-pentadecatrienyl. In some embodiments of any of the aforementioned embodiments, the glyceride polymers include only compounds wherein at least one of R¹, R², R³, and R⁵, or at least one instance of R⁴, is selected from the group consisting of: 8-nonenyl; 8-decenyl; 8-undecenyl; 10-undecenyl; 12-tridecenyl; 8-dodecenyl; 8,11-dodecadienyl; 8,11-tridec adienyl; 8,11-tetradecadienyl; 8,11-pentadecadienyl; 8,11,14-pentadecatrienyl; and 8,11,14-hexadecatrienyl. In some other embodiments of any of the aforementioned embodiments, the glyceride polymers include only compounds wherein at least one of R¹, R², R³, and R⁵, or at least one instance of R⁴, is selected from the group consisting of: 8-nonenyl; 8-decenyl; 8-undecenyl; 8-dodecenyl; 8,11-dodecadienyl; 8,11-tridecadienyl; 8,11-tetradecadienyl; 8,11-pentadecadienyl; 8,11,14-pentadecatrienyl; and 8,11,14-hexadecatrienyl. In some other embodiments of any of the aforementioned embodiments, the glyceride polymers include only compounds wherein at least one of R¹, R², R³, and R⁵, or at least one instance of R⁴, is C₂₋₁₅ alkenyl, or C₂₋₁₄ alkenyl, or C₅₋₁₄ alkenyl, or C₂₋₁₃ alkenyl, or C₂₋₁₂ alkenyl, or C₅₋₁₂ alkenyl.

In a another aspect, glyceride copolymers, which comprises constitutional units formed from reacting two or more monomers in the presence of a metathesis catalyst, the two or more monomers comprise monomer compounds of formula (IIa):

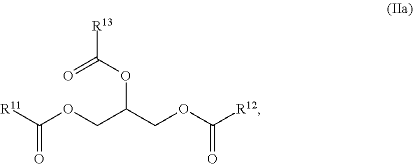

and monomer compounds of formula (IIb):

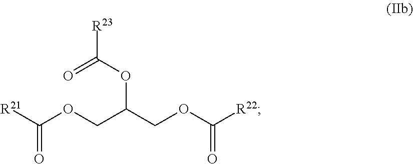

wherein, each R¹¹, R¹², and R¹³ is independently a C₁₋₂₄ alkyl, a substituted C₁₋₂₄ alkyl wherein the substituent is one or more —OH moieties, a C₂₋₂₄ alkenyl, or a substituted C₂₋₂₄ alkenyl wherein the substituent is one or more —OH moieties with the proviso that at least one of R¹¹, R¹², and R¹³ is a C₂₋₂₄ alkenyl or a substituted C₂₋₂₄ alkenyl wherein the substituent is one or more —OH moieties; each R²¹, R²², and R²³ is independently a C₁₋₂₄ alkyl, a substituted C₁₋₂₄ alkyl wherein the substituent is one or more —OH moieties, a C₂₋₂₄ alkenyl, or a substituted C₂₋₂₄ alkenyl wherein the substituent is one or more —OH moieties, with the proviso that at least one of R²¹, R²², and R²³ is 8-nonenyl; 8-decenyl; 8-undecenyl; 8-dodecenyl; 8,11-dodecadienyl; 8,11-tridecadienyl; 8,11-tetradecadienyl; 8,11-pentadecadienyl; 8,11,14-pentadecatrienyl; 8,11,14-hexadecatrienyl; 8,11,14-octadecatrienyl; 9-methyl-8-decenyl; 9-methyl-8-undecenyl; 10-methyl-8-undecenyl; 12-methyl-8,11-tridecadienyl; 12-methyl-8,11-tetradecadienyl; 13-methyl-8,11-tetradecadienyl; 15-methyl-8,11,14-hexadecatrienyl; 15-methyl-8,11,14-heptadecatrienyl; 16-methyl-8,11,14-heptadecatrienyl; 12-tridecenyl; 12-tetradecenyl; 12-pentadecenyl; 12-hexadecenyl; 13-methyl-12-tetradecenyl; 13-methyl-12-pentadecenyl; and 14-methyl-12-pentadecenyl.

The variables R¹¹, R¹², and R¹³ can have any suitable value. In some embodiments, R¹¹, R¹², and R¹³ are independently C₁₋₂₄ alkyl, or C₁₁₋₂₄ alkyl, or C₁₃₋₂₄ alkyl, or C₁₅₋₂₄ alkyl. In some such embodiments, R¹¹, R¹², and R¹³ are independently undecyl, tridecyl, pentadecyl, or heptadecyl. In some further such embodiments, R¹¹, R¹², and R¹³ are independently pentadecyl or heptadecyl. In some embodiments of any of the aforementioned embodiments, R¹¹, R¹², and R¹³ are independently C₂₋₂₄ alkenyl, or C₉₋₂₄ alkenyl, or C₁₁₋₂₄ alkenyl, or C₁₃₋₂₄ alkenyl, or C₁₅₋₂₄ alkenyl. In some such embodiments, R¹¹, R¹², and R¹³ are independently 8-heptadecenyl, 10-heptadecenyl, 8,11-heptadecadienyl or 8,11,14-heptadecatrienyl. In some further such embodiments, R¹¹, R¹², and R¹³ are independently 8-heptadecenyl, 8,11-heptadecadienyl, or 8,11,14-heptadecatrienyl.

The variables R²¹, R²², and R²³ can have any suitable value. In some embodiments of any of the foregoing embodiments, zero, one, or two of R²¹, R²², and R²³ are independently C₁₋₂₄ alkyl, or C₁₁₋₂₄ alkyl, or C₁₃₋₂₄ alkyl, or C₁₅₋₂₄ alkyl. In some such embodiments, zero, one, or two of R²¹, R²², and R²³ are independently undecyl, tridecyl, pentadecyl, or heptadecyl. In some further such embodiments, zero, one, or two of R²¹, R²², and R²³ are independently pentadecyl or heptadecyl. In some embodiments of any of the aforementioned embodiments, zero, one, or two of R²¹, R²², and R²³ are independently C₂₋₂₄ alkenyl, or C₉₋₂₄ alkenyl, or C₁₁₋₂₄ alkenyl, or C₁₃₋₂₄ alkenyl, or C₁₅₋₂₄ alkenyl. In some such embodiments, zero, one, or two of R²¹, R²², and R²³ are independently 8-heptadecenyl, 10-heptadecenyl, 8,11-heptadecadienyl or 8,11,14-heptadecatrienyl. In some further such embodiments, zero, one, or two of R²¹, R²², and R²³ are independently 8-heptadecenyl, 8,11-heptadecadienyl, or 8,11,14-heptadecatrienyl.

In some other embodiments of any of the foregoing embodiments, one, two, or three of R²¹, R²², and R²³ are independently C₂₋₁₅ alkenyl, or C₂₋₁₄ alkenyl, C₅₋₁₄ alkenyl, or C₂₋₁₃ alkenyl, or C₂₋₁₂ alkenyl, or C₅₋₁₂ alkenyl. In some such embodiments, one, two, or three of R²¹, R²², and R²³ are independently 8-nonenyl, 8-decenyl, 8-undecenyl, 8-dodecenyl, 8,11-dodecadienyl, 8,11-tridecadienyl, 8,11-tetradecadienyl, 8,11-pentadecadienyl, 8,11,14-pentadecatrienyl, 8,11,14-hexadecatrienyl, 8,11,14-octadecatrienyl, 9-methyl-8-decenyl, 9-methyl-8-undecenyl, 10-methyl-8-undecenyl, 12-methyl-8,11-tridecadienyl, 12-methyl-8,11-tetradecadienyl, 13-methyl-8,11-tetradecadienyl, 15-methyl-8,11,14-hexadecatrienyl, 15-methyl-8,11,14-heptadecatrienyl, 16-methyl-8,11,14-heptadecatrienyl, 12-tridecenyl, 12-tetradecenyl, 12-pentadecenyl, 12-hexadecenyl, 13-methyl-12-tetradecenyl, 13-methyl-12-pentadecenyl, and 14-methyl-12-pentadecenyl, 10-undecenyl, 8,11,14-heptadecatrienyl, or 8,11,14-octadecatrienyl. In some further such embodiments, one, two, or three of R²¹, R²², and R²³ are independently 8-nonenyl, 8-decenyl, 8-undecenyl, 8-dodecenyl, 8,11-dodecadienyl, 8,11-tridecadienyl, 8,11-tetradecadienyl, 8,11-pentadecadienyl, 8,11,14-pentadecatrienyl, 8,11,14-hexadecatrienyl, 8,11,14-heptadecatrienyl, or 8,11,14-octadecatrienyl. In some further such embodiments, one, two, or three of R²¹, R²², and R²³ are independently 8-nonenyl, 8-undecenyl, 8,11-dodecadienyl, 8,11-tetradecadienyl, or 8,11,14-pentadecatrienyl.

The glyceride copolymers disclosed herein can have any suitable molecular weight. In some embodiments of any of the aforementioned embodiments, the glyceride copolymer has a weight average molecular weight ranging from 4,000 g/mol to 150,000 g/mol, or from 5,000 g/mol to 130,000 g/mol, or from 6,000 g/mol to 100,000 g/mol, or from 7,000 g/mol to 50,000 g/mol, or from 8,000 g/mol to 30,000 g/mol, or from 8,000 g/mol to 20,000 g/mol.

In some embodiments, the glyceride copolymer has a number-average molecular weight (Me) from 2,000 g/mol to 150,000 g/mol, or from 3,000 g/mol to 30,000 g/mol, or from 4,000 g/mol to 20,000 g/mol.

The glyceride copolymers disclosed herein can have any suitable ratio of constitutional units formed from monomer compounds of formula (IIa) to constitutional units formed from monomer compounds of formula (IIb). In some embodiments of any of the aforementioned embodiments, the number ratio of constitutional units formed from monomer compounds of formula (IIa) to constitutional units formed from monomer compounds of formula (IIb) is no more than 10:1, or no more than 9:1, or no more than 8:1, or no more than 7:1, or no more than 6:1, or no more than 5:1, or no more than 4:1, or no more than 3:1, or no more than 2:1, or no more than 1:1. The glyceride copolymers disclosed herein can include additional constitutional units not formed from monomer compounds of either formula (IIa) or formula (IIb), including, but not limited to, constitutional units formed from other unsaturated polyol esters, such as unsaturated diols, triols, and the like.

Or, in some other embodiments of any of the foregoing embodiments, the two or more monomers are reacted in the presence of the metathesis catalyst as part of a reaction mixture, wherein the weight-to-weight ratio of the monomer compounds of formula (IIa) to the monomer compounds of formula (IIb) in the reaction mixture is no more than 10:1, or no more than 9:1, or no more than 8:1, or no more than 7:1, or no more than 6:1, or no more than 5:1, or no more than 4:1, or no more than 3:1, or no more than 2:1, or no more than 1:1. In some embodiments, the reaction mixture includes additional monomer compounds besides monomer compounds of formula (IIa) and formula (IIb).

Any suitable metathesis catalyst can be used, as described in more detail below. In some embodiments of any of the aforementioned embodiments, the metathesis catalyst is an organoruthenium compound, an organoosmium compound, an organotungsten compound, or an organomolybdenum compound.

In a another aspect, the disclosure provides glyceride copolymers, which comprises constitutional units formed from reacting two or more monomers in the presence of a first metathesis catalyst; wherein the first monomer is an unsaturated natural oil glyceride, and the second monomer is an unsaturated alkenylized natural oil glyceride. In a another aspect, the disclosure provides glyceride copolymers, which comprises constitutional units formed from reacting two or more monomers in the presence of a first metathesis catalyst; wherein the first monomer is an unsaturated synthetic polyol ester, and the second monomer is an unsaturated alkenylized natural oil glyceride. In a another aspect, the disclosure provides glyceride copolymers, which comprises constitutional units formed from reacting two or more monomers in the presence of a first metathesis catalyst; wherein the first monomer is an unsaturated natural oil glyceride, and the second monomer is an unsaturated alkenylized synthetic polyol ester. In a another aspect, the disclosure provides glyceride copolymers, which comprises constitutional units formed from reacting two or more monomers in the presence of a first metathesis catalyst; wherein the first monomer is an unsaturated synthetic polyol ester, and the second monomer is an unsaturated alkenylized synthetic polyol ester. In a another aspect, the disclosure provides glyceride copolymers, which comprises constitutional units formed from reacting two or more monomers in the presence of a first metathesis catalyst; wherein the first monomer is a first unsaturated alkenylized synthetic polyol ester, and the second monomer is a second unsaturated alkenylized synthetic polyol ester. In a another aspect, the disclosure provides glyceride copolymers, which comprises constitutional units formed from reacting two or more monomers in the presence of a first metathesis; wherein the first monomer is a first unsaturated alkenylized natural oil glyceride, and the second monomer is a second unsaturated alkenylized natural oil glyceride. In a another aspect, the disclosure provides glyceride copolymers, which comprises constitutional units formed from reacting two or more monomers in the presence of a first metathesis; wherein the first monomer is an unsaturated alkenylized natural oil glyceride, and the second monomer is an unsaturated alkenylized synthetic polyol ester.

In some embodiments, the unsaturated alkenylized natural oil glyceride is formed from the reaction of a second unsaturated natural oil glyceride with a short-chain alkene in the presence of a second metathesis catalyst. In some such embodiments, the unsaturated alkenylized natural oil glyceride has a lower molecular weight than the second unsaturated natural oil glyceride. Any suitable short-chain alkene can be used, according to the embodiments described above. In some embodiments, the short-chain alkene is a C₂₋₈ olefin, or a C₂₋₆ olefin. In some such embodiments, the short-chain alkene is ethylene, propylene, 1-butene, 2-butene, isobutene, 1-pentene, 2-pentene, 1-hexene, 2-hexene, or 3-hexene. In some further such embodiments, the short-chain alkene is ethylene, propylene, 1-butene, 2-butene, or isobutene. In some embodiments, the short-chain alkene is ethylene. In some embodiments, the short-chain alkene is propylene. In some embodiments, the short-chain alkene is 1-butene. In some embodiments, the short-chain alkene is 2-butene. In some other embodiments, the short-chain alkene is a branched short-chain alkene. Non-limiting examples of such branched short-chain alkenes include, but are not limited to, isobutylene, 3-methyl-1-butene, 3-methyl-1-pentene, and 4-methyl-1-pentene.

The unsaturated natural oil glyceride can be obtained from any suitable natural oil source. In some embodiments of any of the aforementioned embodiments, the unsaturated natural oil glycerides are obtained from synthesized oils, natural oils (e.g., vegetable oils, algae oils, bacterial derived oils, and animal fats), combinations of these, and the like. In some embodiments, the natural oil is obtained from a vegetable oil, such as a seed oil. Recycled used vegetable oils may also be used. In some further embodiments, the vegetable oil is Abyssinian oil, Almond Oil, Apricot Oil, Apricot Kernel oil, Argan oil, Avocado Oil, Babassu Oil, Baobab Oil, Black Cumin Oil, Black Currant Oil, Borage Oil, Camelina oil, Carinata oil, Canola (low erucic acid rapeseed) oil, Castor oil, Cherry Kernel Oil, Coconut oil, Corn oil, Cottonseed oil, Echium Oil, Evening Primrose Oil, Flax Seed Oil, Grape Seed Oil, Grapefruit Seed Oil, Hazelnut Oil, Hemp Seed Oil, Jatropha oil, Jojoba Oil, Kukui Nut Oil, Linseed Oil, Macadamia Nut Oil, Meadowfoam Seed Oil, Moringa Oil, Mustard Seed Oil, Neem Oil, Olive Oil, Palm Oil, Palm Kernel Oil, Peach Kernel Oil, Peanut Oil, Pecan Oil, Pennycress oil, Perilla Seed Oil, Pistachio Oil, Pomegranate Seed Oil, Pongamia oil, Pumpkin Seed Oil, Raspberry Oil, Red Palm Olein, Rice Bran Oil, Rosehip Oil, Safflower Oil, Seabuckthorn Fruit Oil, Sesame Seed Oil, Shea Olein, Sunflower Oil, Soybean Oil, Tonka Bean Oil, Tung Oil, Walnut Oil, Wheat Germ Oil, High Oleoyl Soybean Oil, High Oleoyl Sunflower Oil, High Oleoyl Safflower Oil, High Erucic Acid Rapeseed Oil, and mixtures thereof. In some embodiments, the vegetable oil is palm oil. In some embodiments, the vegetable oil is soybean oil. In some embodiments, the vegetable oil is canola oil. In some embodiments, a representative, non-limiting example of animal fat is lard, tallow, chicken fat, yellow grease, fish oil, emu oil, combinations of these, and the like. In some embodiments, a representative non-limiting example of a synthesized oil includes tall oil, which is a byproduct of wood pulp manufacture. In some embodiments, the natural oil is refined, bleached, and/or deodorized.

Natural oils of the type described herein typically are composed of triglycerides of fatty acids. These fatty acids may be either saturated, monounsaturated or polyunsaturated and contain varying chain lengths ranging from C₈ to C₃₀. The most common fatty acids include saturated fatty acids such as lauric acid (dodecanoic acid), myristic acid (tetradecanoic acid), palmitic acid (hexadecanoic acid), stearic acid (octadecanoic acid), arachidic acid (eicosanoic acid), and lignoceric acid (tetracosanoic acid); unsaturated acids include such fatty acids as palmitoleic (a C₁₆ acid), and oleic acid (a C₁₈ acid); polyunsaturated acids include such fatty acids as linoleic acid (a di-unsaturated C₁₈ acid), linolenic acid (a tri-unsaturated C₁₈ acid), and arachidonic acid (a tetra-unsubstituted C₂₀ acid). The natural oils are further comprised of esters of these fatty acids in random placement onto the three sites of the trifunctional glycerine molecule. Different natural oils will have different ratios of these fatty acids, and within a given natural oil there is a range of these acids as well depending on such factors as where a vegetable or crop is grown, maturity of the vegetable or crop, the weather during the growing season, etc. Thus, it is difficult to have a specific or unique structure for any given natural oil, but rather a structure is typically based on some statistical average. For example soybean oil contains a mixture of predominantly C16 and C18 acid groups where stearic acid, oleic acid, linoleic acid, and linolenic acid are in the ratio of about 15:24:50:11, and an average number of double bonds of 4.4-4.7 per triglyceride. One method of quantifying the number of double bonds is the iodine value (IV) which is defined as the number of grams of iodine that will react with 100 grams of oil. Therefore for soybean oil, the average iodine value range is from 120-140. Soybean oil may comprise about 95% by weight or greater (e.g., 99% weight or greater) triglycerides of fatty acids. Major fatty acids in the polyol esters of soybean oil include saturated fatty acids, as a non-limiting example, palmitic acid (hexadecanoic acid) and stearic acid (octadecanoic acid), and unsaturated fatty acids, as a non-limiting example, oleic acid (9-octadecenoic acid), linoleic acid (9,12octadecadienoic acid), and linolenic acid (9,12,15-octadecatrienoic acid).

In an exemplary embodiment, the vegetable oil is canola oil, for example, refined, bleached, and deodorized canola oil (i.e., RBD canola oil). Canola oil is an unsaturated polyol ester of glycerol that typically comprises about 95% weight or greater (e.g., 99% weight or greater) triglycerides of fatty acids. Major fatty acids in the polyol esters of canola oil include saturated fatty acids, for example, palmitic acid (hexadecanoic acid) and stearic acid (octadecanoic acid), and unsaturated fatty acids, for example, oleic acid (9-octadecenoic acid), linoleic acid (9,12-octadecadienoic acid), and linolenic acid (9,12,15-octadecatrienoic acid). Canola oil is a highly unsaturated vegetable oil with many of the triglyceride molecules having at least two unsaturated fatty acids (i.e., a polyunsaturated triglyceride).

In some embodiments, the unsaturated alkenylized synthetic polyol ester is formed from the reaction of an unsaturated synthetic polyol ester with a short-chain alkene in the presence of a second metathesis catalyst. In some such embodiments, the unsaturated alkenylized synthetic polyol ester has a lower molecular weight than the second unsaturated synthetic polyol ester. Any suitable short-chain alkene can be used, according to the embodiments described above. In some embodiments, the short-chain alkene is a C₂₋₈ olefin, or a C₂₋₆ olefin. In some such embodiments, the short-chain alkene is ethylene, propylene, 1-butene, 2-butene, isobutene, 1-pentene, 2-pentene, 1-hexene, 2-hexene, or 3-hexene. In some further such embodiments, the short-chain alkene is ethylene, propylene, 1-butene, 2-butene, or isobutene. In some embodiments, the short-chain alkene is ethylene. In some embodiments, the short-chain alkene is propylene. In some embodiments, the short-chain alkene is 1-butene. In some embodiments, the short-chain alkene is 2-butene. In some other embodiments, the short-chain alkene is a branched short-chain alkene. Non-limiting examples of such branched short-chain alkenes include, but are not limited to, isobutylene, 3-methyl-1-butene, 3-methyl-1-pentene, and 4-methyl-1-pentene.

The unsaturated synthetic polyol ester includes esters such as those derived from ethylene glycol or propylene glycol, polyethylene glycol, polypropylene glycol, or poly(tetramethylene ether) glycol, esters such as those derived from pentaerythritol, dipentaerythritol, tripentaerythritol, trimethylolpropane, or neopentyl glycol, or sugar esters such as SEFOSE®. Sugar esters such as SEFOSE® include one or more types of sucrose polyesters, with up to eight ester groups that could undergo a metathesis exchange reaction. Sucrose polyesters are derived from a natural resource and therefore, the use of sucrose polyesters can result in a positive environmental impact. Sucrose polyesters are polyester materials, having multiple substitution positions around the sucrose backbone coupled with the chain length, saturation, and derivation variables of the fatty chains. Such sucrose polyesters can have an esterification (“IBAR”) of greater than about 5. In one embodiment the sucrose polyester may have an IBAR of from about 5 to about 8. In another embodiment the sucrose polyester has an IBAR of about 5-7, and in another embodiment the sucrose polyester has an IBAR of about 6. In yet another embodiment the sucrose polyester has an IBAR of about 8. As sucrose polyesters are derived from a natural resource, a distribution in the IBAR and chain length may exist. For example a sucrose polyester having an IBAR of 6, may contain a mixture of mostly IBAR of about 6, with some IBAR of about 5 and some IBAR of about 7. Additionally, such sucrose polyesters may have an unsaturation or iodine value (“IV”) of about 3 to about 140. In another embodiment the sucrose polyester may have an IV of about 10 to about 120. In yet another embodiment the sucrose polyester may have an IV of about 20 to 100. Further, such sucrose polyesters have a chain length of about C₁₂₋₂₀ but are not limited to these chain lengths.

Non-limiting examples of sucrose polyesters suitable for use include SEFOSE® 1618S, SEFOSE® 1618U, SEFOSE® 1618H, Sefa Soyate IMF 40, Sefa Soyate LP426, SEFOSE® 2275, SEFOSE® C1695, SEFOSE® C18:0 95, SEFOSE® C1495, SEFOSE® 1618H B6, SEFOSE® 1618S B6, SEFOSE® 1618U B6, Sefa Cottonate, SEFOSE® C1295, Sefa C895, Sefa C1095, SEFOSE® 1618S B4.5, all available from The Procter and Gamble Co. of Cincinnati, Ohio.

Other examples of suitable unsaturated polyol esters may include but not be limited to sorbitol esters, maltitol esters, sorbitan esters, maltodextrin derived esters, xylitol esters, polyglycerol esters, and other sugar derived esters.

The glyceride copolymers disclosed herein can have any suitable molecular weight. In some embodiments of any of the aforementioned embodiments, the glyceride copolymer has a weight average molecular weight ranging from 4,000 g/mol to 150,000 g/mol, or from 5,000 g/mol to 130,000 g/mol, or from 6,000 g/mol to 100,000 g/mol, or from 7,000 g/mol to 50,000 g/mol, or from 8,000 g/mol to 30,000 g/mol, or from 8,000 g/mol to 20,000 g/mol.

In some embodiments, the glyceride copolymer has a number-average molecular weight (Me) from 2,000 g/mol to 150,000 g/mol, or from 3,000 g/mol to 30,000 g/mol, or from 4,000 g/mol to 20,000 g/mol.

The glyceride copolymers disclosed herein can have any suitable ratio of constitutional units formed from the first monomer to constitutional units formed from the second monomer. In some embodiments of any of the aforementioned embodiments, the number ratio of constitutional units formed from the first monomer to constitutional units formed from the second monomer is no more than 10:1, or no more than 9:1, or no more than 8:1, or no more than 7:1, or no more than 6:1, or no more than 5:1, or no more than 4:1, or no more than 3:1, or no more than 2:1, or no more than 1:1. The glyceride copolymers disclosed herein can include additional constitutional units not formed from the first monomer or the second monomer, including, but not limited to, constitutional units formed from other unsaturated polyol esters, such as unsaturated diols, triols, and the like.

Or, in some other embodiments of any of the foregoing embodiments, the two or more monomers are reacted in the presence of the metathesis catalyst as part of a reaction mixture, wherein the weight-to-weight ratio of the first monomer to the second monomer in the reaction mixture is no more than 10:1, or no more than 9:1, or no more than 8:1, or no more than 7:1, or no more than 6:1, or no more than 5:1, or no more than 4:1, or no more than 3:1, or no more than 2:1, or no more than 1:1. In some embodiments, the reaction mixture includes additional monomer compounds besides the first monomer and the second monomer.

Any suitable metathesis catalyst can be used as either the first metathesis catalyst or the second metathesis catalyst, as described in more detail below. In some embodiments of any of the aforementioned embodiments, the first and second metathesis catalysts are an organoruthenium compound, an organoosmium compound, an organo-tungsten compound, or an organomolybdenum compound.

Additional glyceride copolymers are contemplated as products of the synthetic methods and examples disclosed herein.

### Synthetic Methods

In a fifth aspect, the disclosure provides methods of forming a glyceride copolymer composition, the methods comprising: (a) providing a reaction mixture comprising a metathesis catalyst and monomer compounds of formula (IIIa):

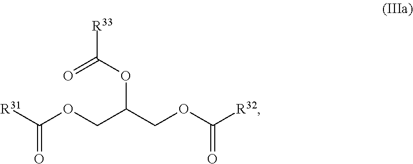

and monomer compounds of formula (IIIb):

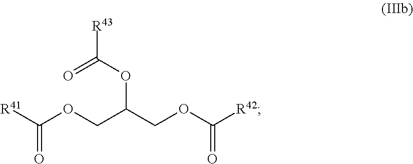

wherein, R³¹, R³², and R³³ are independently C₁₋₂₄ alkyl or C₂₋₂₄ alkenyl, each of which is optionally substituted one or more times by —OH, provided that at least one of R³¹, R³², and R³³ is C₂₋₂₄ alkenyl, which is optionally substituted one or more times by —OH; and R⁴¹, R⁴², and R⁴³ are independently C₁₋₂₄ alkyl or C₂₋₂₄ alkenyl, each of which is optionally substituted one or more times by —OH, provided that at least one of R⁴¹, R⁴², and R⁴³ is 8-nonenyl, 8-decenyl, 8-undecenyl, 8-dodecenyl, 8,11-dodecadienyl, 8,11-tridecadienyl, 8,11-tetradecadienyl, 8,11-pentadecadienyl, 8,11,14-pentadecatrienyl, 8,11,14-hexadecatrienyl, 8,11,14-heptadecatrienyl, or 8,11,14-octadecatrienyl; and (b) reacting the monomer compounds of formula (IIIa) with the monomer compounds of formula (IIIb) in the presence of the metathesis catalyst to form the glyceride polymer composition.

The variables R³¹, R³², and R³³ can have any suitable value. In some embodiments, R³¹, R³², and R³³ are independently C₁₋₂₄ alkyl, or C₁₁₋₂₄ alkyl, or C₁₃₋₂₄ alkyl, or C₁₅₋₂₄ alkyl. In some such embodiments, R³¹, R³², and R³³ are independently undecyl, tridecyl, pentadecyl, or heptadecyl. In some further such embodiments, R³¹, R³², and R³³ are independently pentadecyl or heptadecyl. In some embodiments of any of the aforementioned embodiments, R³¹, R³², and R³³ are independently C₂₋₂₄ alkenyl, or C₉₋₂₄ alkenyl, or C₁₁₋₂₄ alkenyl, or C₁₃₋₂₄ alkenyl, or C₁₅₋₂₄ alkenyl. In some such embodiments, R³¹, R³², and R³³ are independently 8-heptadecenyl, 10-heptadecenyl, 8,11-heptadecadienyl or 8,11,14-heptadecatrienyl. In some further such embodiments, R³¹, R³², and R³³ are independently 8-heptadecenyl, 8,11-heptadecadienyl, or 8,11,14-heptadecatrienyl.

The variables R⁴¹, R⁴², and R⁴³ can have any suitable value. In some embodiments of any of the foregoing embodiments, zero, one, or two of R⁴¹, R⁴², and R⁴³ are independently C₁₋₂₄ alkyl, or C₁₁₋₂₄ alkyl, or C₁₃₋₂₄ alkyl, or C₁₅₋₂₄ alkyl. In some such embodiments, zero, one, or two of R⁴¹, R⁴², and R⁴³ are independently undecyl, tridecyl, pentadecyl, or heptadecyl. In some further such embodiments, zero, one, or two of R⁴¹, R⁴², and R⁴³ are independently pentadecyl or heptadecyl. In some embodiments of any of the aforementioned embodiments, zero, one, or two of R⁴¹, R⁴², and R⁴³ are independently C₂₋₂₄ alkenyl, or C₉₋₂₄ alkenyl, or C₁₁₋₂₄ alkenyl, or C₁₃₋₂₄ alkenyl, or C₁₅₋₂₄ alkenyl. In some such embodiments, zero, one, or two of R⁴¹, R⁴², and R⁴³ are independently 8-heptadecenyl, 10-heptadecenyl, 8,11-heptadecadienyl or 8,11,14-heptadecatrienyl. In some further such embodiments, zero, one, or two of R⁴¹, R⁴², and R⁴³ are independently 8-heptadecenyl, 8,11-heptadecadienyl, or 8,11,14-heptadecatrienyl.

In some other embodiments of any of the foregoing embodiments, one, two, or three of R⁴¹, R⁴², and R⁴³ are independently C₂₋₁₅ alkenyl, or C₂₋₁₄ alkenyl, or C₂₋₁₃ alkenyl, or C₂₋₁₂ alkenyl, or C₅₋₁₂ alkenyl. In some such embodiments, one, two, or three of R⁴¹, R⁴², and R⁴³ are independently 8-nonenyl, 8-decenyl, 8-undecenyl, 10-undecenyl, 8-dodecenyl, 8,11-dodecadienyl, 8,11-tridecadienyl, 8,11-tetradecadienyl, 8,11-pentadecadienyl, 8,11,14-pentadecatrienyl, 8,11,14-hexadecatrienyl, 8,11,14-heptadecatrienyl, or 8,11,14-octadecatrienyl. In some further such embodiments, one, two, or three of R⁴¹, R⁴², and R⁴³ are independently 8-nonenyl, 8-decenyl, 8-undecenyl, 8-dodecenyl, 8,11-dodecadienyl, 8,11-tridecadienyl, 8,11-tetradecadienyl, 8,11-pentadecadienyl, 8,11,14-pentadecatrienyl, 8,11,14-hexadecatrienyl, 8,11,14-heptadecatrienyl, or 8,11,14-octadecatrienyl. In some further such embodiments, one, two, or three of R⁴¹, R⁴², and R⁴³ are independently 8-nonenyl, 8-undecenyl, 8,11-dodecadienyl, 8,11-tetradecadienyl, or 8,11,14-pentadecatrienyl.

The glyceride copolymers formed by the methods disclosed herein can have any suitable molecular weight. In some embodiments of any of the aforementioned embodiments, the glyceride copolymer has a weight average molecular weight ranging from 4,000 g/mol to 150,000 g/mol, or from 5,000 g/mol to 130,000 g/mol, or from 6,000 g/mol to 100,000 g/mol, or from 7,000 g/mol to 50,000 g/mol, or from 8,000 g/mol to 30,000 g/mol, or from 8,000 g/mol to 20,000 g/mol.

The glyceride copolymers formed by the methods disclosed herein can have any suitable ratio of constitutional units formed from monomer compounds of formula (IIIa) to constitutional units formed from monomer compounds of formula (IIIb). In some embodiments of any of the aforementioned embodiments, the number ratio of constitutional units formed from monomer compounds of formula (IIIa) to constitutional units formed from monomer compounds of formula (IIIb) is no more than 10:1, or no more than 9:1, or no more than 8:1, or no more than 7:1, or no more than 6:1, or no more than 5:1, or no more than 4:1, or no more than 3:1, or no more than 2:1, or no more than 1:1. The glyceride copolymers disclosed herein can include additional constitutional units not formed from monomer compounds of either formula (IIIa) or formula (IIIb).

Or, in some other embodiments of any of the foregoing embodiments, the two or more monomers are reacted in the presence of the metathesis catalyst as part of a reaction mixture, wherein the weight-to-weight ratio of the monomer compounds of formula (IIIa) to the monomer compounds of formula (IIIb) in the reaction mixture is no more than 10:1, or no more than 9:1, or no more than 8:1, or no more than 7:1, or no more than 6:1, or no more than 5:1, or no more than 4:1, or no more than 3:1, or no more than 2:1, or no more than 1:1. In some embodiments, the reaction mixture includes additional monomer compounds besides monomer compounds of formula (IIIa) and formula (IIIb).

Any suitable metathesis catalyst can be used, as described in more detail below. In some embodiments of any of the aforementioned embodiments, the metathesis catalyst is an organoruthenium compound, an organoosmium compound, an organotungsten compound, or an organomolybdenum compound.

The methods disclosed herein can include additional chemical and physical treatment of the resulting glyceride copolymers. For example, in some embodiments, the resulting glyceride copolymers are subjected to full or partial hydrogenation, such as diene-selective hydrogenation. Also, in some embodiments, the unspent metathesis catalyst and/or the spent metathesis catalyst residues are recovered. In some embodiments of any of the foregoing embodiments, the resulting glyceride polymers are subjected to methods that induce isomerization, such as olefin isomerization.

In another aspect, the disclosure provides methods of forming a glyceride copolymer, the methods comprising: (a) providing a reaction mixture comprising a first metathesis catalyst, unsaturated natural oil glycerides, and unsaturated alkenylized natural oil glycerides; and (b) reacting the unsaturated natural oil glycerides and unsaturated alkenylized natural oil glycerides in the presence of the first metathesis catalyst to form the glyceride copolymer.

In some embodiments, the unsaturated alkenylized natural oil glyceride is formed from the reaction of a second unsaturated natural oil glyceride with a short-chain alkene in the presence of a second metathesis catalyst. In some such embodiments, the unsaturated alkenylized natural oil glyceride has a lower molecular weight than the second unsaturated natural oil glyceride. Any suitable short-chain alkene can be used, according to the embodiments described above. In some embodiments, the short-chain alkene is a C₂₋₁₄ olefin, C₂₋₁₂ olefin, C₂₋₁₀ olefin, C₂₋₈ olefin, C₂₋₆ olefin, or a C₂₋₄ olefin. In some such embodiments, the short-chain alkene may comprise at least one of the following: ethylene, propylene, 1-butene, 2-butene, isobutene, 1-pentene, 2-pentene, 1-hexene, 2-hexene, 3-hexene, cyclohexene, 2-methyl-1-butene, 2-methyl-2-butene, 3-methyl-1-butene, cyclopentene, 2-methyl-1-pentene, 3-methyl-1-pentene, 4-methyl-1-pentene, 2-methyl-2-pentene, 3-methyl-2-pentene, 4-methyl-2-pentene, or 4,4-dimethyl-2-pentene. In some further such embodiments, the short-chain alkene is ethylene, propylene, 1-butene, 2-butene, or isobutene. In some embodiments, the short-chain alkene is ethylene. In some embodiments, the short-chain alkene is propylene. In some embodiments, the short-chain alkene is 1-butene. In some embodiments, the short-chain alkene is 2-butene.

As noted, it is possible to use a mixture of various linear or branched low-molecular-weight olefins in the reaction to achieve the desired metathesis product distribution. In one embodiment, a mixture of butenes (1-butene, 2-butenes, and, optionally, isobutene) may be employed as the low molecular-weight olefin, offering a low cost, commercially available feedstock instead a purified source of one particular butene. Such low cost mixed butene feedstocks are typically diluted with n-butane and/or isobutane.

The first unsaturated natural oil glyceride and the second unsaturated natural oil glyceride can be obtained from any suitable natural oil source. In some embodiments of any of the aforementioned embodiments, the first or second unsaturated natural oil glycerides are obtained from a vegetable oil, such as a seed oil. In some further embodiments, the vegetable oil is rapeseed oil, canola oil (low erucic acid rapeseed oil), coconut oil, corn oil, cottonseed oil, olive oil, palm oil, peanut oil, safflower oil, sesame oil, soybean oil, sunflower oil, linseed oil, palm kernel oil, tung oil, jatropha oil, mustard seed oil, pennycress oil, camelina oil, hempseed oil, or castor oil. In some embodiments, the vegetable oil is palm oil. In some embodiments, the vegetable oil is soybean oil. In some embodiments, the vegetable oil is canola oil.

The glyceride copolymers formed by the methods disclosed herein can have any suitable molecular weight. In some embodiments of any of the aforementioned embodiments, the glyceride copolymer has a weight average molecular weight ranging from 4,000 g/mol to 150,000 g/mol, or from 5,000 g/mol to 130,000 g/mol, or from 6,000 g/mol to 100,000 g/mol, or from 7,000 g/mol to 50,000 g/mol, or from 8,000 g/mol to 30,000 g/mol, or from 8,000 g/mol to 20,000 g/mol.

In some embodiments, the glyceride copolymer has a number-average molecular weight (Mₙ) from 2,000 g/mol to 150,000 g/mol, or from 3,000 g/mol to 30,000 g/mol, or from 4,000 g/mol to 20,000 g/mol.

The glyceride copolymers formed by the methods disclosed herein can have any suitable ratio of constitutional units formed from the first monomer to constitutional units formed from the second monomer. In some embodiments of any of the aforementioned embodiments, the number ratio of constitutional units formed from the first monomer to constitutional units formed from the second monomer is no more than 10:1, or no more than 9:1, or no more than 8:1, or no more than 7:1, or no more than 6:1, or no more than 5:1, or no more than 4:1, or no more than 3:1, or no more than 2:1, or no more than 1:1. The glyceride copolymers disclosed herein can include additional constitutional units not formed from the first monomer or the second monomer.

Or, in some other embodiments of any of the foregoing embodiments, the two or more monomers are reacted in the presence of the metathesis catalyst as part of a reaction mixture, wherein the weight-to-weight ratio of the first monomer to the second monomer in the reaction mixture is no more than 10:1, or no more than 9:1, or no more than 8:1, or no more than 7:1, or no more than 6:1, or no more than 5:1, or no more than 4:1, or no more than 3:1, or no more than 2:1, or no more than 1:1. In some embodiments, the reaction mixture includes additional monomer compounds besides the first monomer and the second monomer.

Any suitable metathesis catalyst can be used as either the first metathesis catalyst or the second metathesis catalyst, as described in more detail below. In some embodiments of any of the aforementioned embodiments, the first and second metathesis catalysts are an organoruthenium compound, an organoosmium compound, an organo-tungsten compound, or an organomolybdenum compound.

The methods disclosed herein can include additional chemical and physical treatment of the resulting glyceride copolymers. For example, in some embodiments, the resulting glyceride copolymers are subjected to full or partial hydrogenation, such as diene-selective hydrogenation.

Derivation from Renewable Sources

The compounds employed in any of the aspects or embodiments disclosed herein can, in certain embodiments, be derived from renewable sources, such as from various natural oils or their derivatives. Any suitable methods can be used to make these compounds from such renewable sources.

Olefin metathesis provides one possible means to convert certain natural oil feedstocks into olefins and esters that can be used in a variety of applications, or that can be further modified chemically and used in a variety of applications. In some embodiments, a composition (or components of a composition) may be formed from a renewable feedstock, such as a renewable feedstock formed through metathesis reactions of natural oils and/or their fatty acid or fatty ester derivatives. When compounds containing a carbon-carbon double bond undergo metathesis reactions in the presence of a metathesis catalyst, some or all of the original carbon-carbon double bonds are broken, and new carbon-carbon double bonds are formed. The products of such metathesis reactions include carbon-carbon double bonds in different locations, which can provide unsaturated organic compounds having useful chemical properties.

A wide range of natural oils, or derivatives thereof, can be used in such metathesis reactions. Examples of suitable natural oils include, but are not limited to, vegetable oils, algae oils, fish oils, animal fats, tall oils, derivatives of these oils, combinations of any of these oils, and the like. Representative non-limiting examples of vegetable oils include rapeseed oil (canola oil), coconut oil, corn oil, cottonseed oil, olive oil, palm oil, peanut oil, safflower oil, sesame oil, soybean oil, sunflower oil, linseed oil, palm kernel oil, tung oil, jatropha oil, mustard seed oil, pennycress oil, camelina oil, hempseed oil, and castor oil. Representative non-limiting examples of animal fats include lard, tallow, poultry fat, yellow grease, and fish oil. Tall oils are by-products of wood pulp manufacture. In some embodiments, the natural oil or natural oil feedstock comprises one or more unsaturated glycerides (e.g., unsaturated triglycerides). In some such embodiments, the natural oil feedstock comprises at least 50% by weight, or at least 60% by weight, or at least 70% by weight, or at least 80% by weight, or at least 90% by weight, or at least 95% by weight, or at least 97% by weight, or at least 99% by weight of one or more unsaturated triglycerides, based on the total weight of the natural oil feedstock.

The natural oil may include canola or soybean oil, such as refined, bleached and deodorized soybean oil (i.e., RBD soybean oil). Soybean oil typically includes about 95 percent by weight (wt %) or greater (e.g., 99 wt % or greater) triglycerides of fatty acids. Major fatty acids in the polyol esters of soybean oil include but are not limited to saturated fatty acids such as palmitic acid (hexadecanoic acid) and stearic acid (octadecanoic acid), and unsaturated fatty acids such as oleic acid (9-octadecenoic acid), linoleic acid (9,12-octadecadienoic acid), and linolenic acid (9,12,15-octadecatrienoic acid).

Such natural oils, or derivatives thereof, contain esters, such as triglycerides, of various unsaturated fatty acids. The identity and concentration of such fatty acids varies depending on the oil source, and, in some cases, on the variety. In some embodiments, the natural oil comprises one or more esters of oleic acid, linoleic acid, linolenic acid, or any combination thereof. When such fatty acid esters are metathesized, new compounds are formed. For example, in embodiments where the metathesis uses certain short-chain alkenes, e.g., ethylene, propylene, or 1-butene, and where the natural oil includes esters of oleic acid, an amount of 1-decene and 1-decenoid acid (or an ester thereof), among other products, are formed.

In some embodiments, the natural oil can be subjected to various pre-treatment processes, which can facilitate their utility for use in certain metathesis reactions. Useful pre-treatment methods are described in United States Patent Application Publication Nos. 2011/0113679, 2014/0275595, and 2014/0275681, all three of which are hereby incorporated by reference as though fully set forth herein.

In certain embodiments, prior to the metathesis reaction, the natural oil and/or unsaturated polyol ester feedstock may be treated to render the natural oil more suitable for the subsequent metathesis reaction. In one embodiment, the treatment of the natural oil and/or unsaturated polyol ester involves the removal of catalyst poisons, such as peroxides, which may potentially diminish the activity of the metathesis catalyst. Non-limiting examples of the natural oil and/or unsaturated polyol ester feedstock treatment methods to diminish catalyst poisons include those described in PCT/US2008/09604, PCT/US2008/09635, and U.S. patent application Ser. Nos. 12/672,651 and 12/672,652, herein incorporated by reference in their entireties. In certain embodiments, the natural oil and/or unsaturated polyol ester feedstock is thermally treated by heating the feedstock to a temperature greater than 100° C. in the absence of oxygen and held at the temperature for a time sufficient to diminish catalyst poisons in the feedstock. In other embodiments, the temperature is between approximately 100° C. and 300° C., between approximately 120° C. and 250° C., between approximately 150° C. and 210° C., or approximately between 190 and 200° C. In one embodiment, the absence of oxygen is achieved by sparging the natural oil and/or unsaturated polyol ester feedstock with nitrogen, wherein the nitrogen gas is pumped into the feedstock treatment vessel at a pressure of approximately 10 atm (150 psig).

In certain embodiments, the natural oil and/or unsaturated polyol ester feedstock is chemically treated under conditions sufficient to diminish the catalyst poisons in the feedstock through a chemical reaction of the catalyst poisons. In certain embodiments, the feedstock is treated with a reducing agent or a cation-inorganic base composition. Non-limiting examples of reducing agents include bisulfate, borohydride, phosphine, thiosulfate, and combinations thereof.

In certain embodiments, the natural oil and/or unsaturated polyol ester feedstock is treated with an adsorbent to remove catalyst poisons. In one embodiment, the feedstock is treated with a combination of thermal and adsorbent methods. In another embodiment, the feedstock is treated with a combination of chemical and adsorbent methods. In another embodiment, the treatment involves a partial hydrogenation treatment to modify the natural oil and/or unsaturated polyol ester feedstocks reactivity with the metathesis catalyst. Additional non-limiting examples of feedstock treatment are also described below when discussing the various metathesis catalysts.

In some embodiments, after any optional pre-treatment of the natural oil feedstock, the natural oil feedstock is reacted in the presence of a metathesis catalyst in a metathesis reactor. In some other embodiments, an unsaturated ester (e.g., an unsaturated glyceride, such as an unsaturated triglyceride) is reacted in the presence of a metathesis catalyst in a metathesis reactor. These unsaturated esters may be a component of a natural oil feedstock, or may be derived from other sources, e.g., from esters generated in earlier-performed metathesis reactions.

In some embodiments, the natural oil is winterized. Winterization refers to the process of: (1) removing waxes and other non-triglyceride constituents, (2) removing naturally occurring high-melting triglycerides, and (3) removing high-melting triglycerides formed during partial hydrogenation. Winterization may be accomplished by known methods including, for example, cooling the oil at a controlled rate in order to cause crystallization of the higher melting components that are to be removed from the oil. The crystallized high melting components are then removed from the oil by filtration resulting in winterized oil. Winterized soybean oil is commercially available from Cargill, Incorporated (Minneapolis, Minn.).

The conditions for such metathesis reactions, and the reactor design, and suitable catalysts are as described below with reference to the metathesis of the olefin esters. That discussion is incorporated by reference as though fully set forth herein.

### Olefin Metathesis

In some embodiments, one or more of the unsaturated monomers can be made by metathesizing a natural oil or natural oil derivative. The terms “metathesis” or “metathesizing” can refer to a variety of different reactions, including, but not limited to, cross-metathesis, self-metathesis, ring-opening metathesis, ring-opening metathesis polymerizations (“ROMP”), ring-closing metathesis (“RCM”), and acyclic diene metathesis (“ADMET”). Any suitable metathesis reaction can be used, depending on the desired product or product mixture.

In some embodiments, after any optional pre-treatment of the natural oil feedstock, the natural oil feedstock is reacted in the presence of a metathesis catalyst in a metathesis reactor. In some other embodiments, an unsaturated ester (e.g., an unsaturated glyceride, such as an unsaturated triglyceride) is reacted in the presence of a metathesis catalyst in a metathesis reactor. These unsaturated esters may be a component of a natural oil feedstock, or may be derived from other sources, e.g., from esters generated in earlier-performed metathesis reactions. In certain embodiments, in the presence of a metathesis catalyst, the natural oil or unsaturated ester can undergo a self-metathesis reaction with itself.

In some embodiments, the metathesis comprises reacting a natural oil feedstock (or another unsaturated ester) in the presence of a metathesis catalyst. In some such embodiments, the metathesis comprises reacting one or more unsaturated glycerides (e.g., unsaturated triglycerides) in the natural oil feedstock in the presence of a metathesis catalyst. In some embodiments, the unsaturated glyceride comprises one or more esters of oleic acid, linoleic acid, linoleic acid, or combinations thereof. In some other embodiments, the unsaturated glyceride is the product of the partial hydrogenation and/or the metathesis of another unsaturated glyceride (as described above).

In some embodiments, the unsaturated polyol ester is partially hydrogenated before being metathesized. For example, in some embodiments, the unsaturated polyol ester is partially hydrogenated to achieve an iodine value (IV) of about 120 or less before subjecting the partially hydrogenated polyol ester to metathesis.

The metathesis process can be conducted under any conditions adequate to produce the desired metathesis products. For example, stoichiometry, atmosphere, solvent, temperature, and pressure can be selected by one skilled in the art to produce a desired product and to minimize undesirable byproducts. In some embodiments, the metathesis process may be conducted under an inert atmosphere. Similarly, in embodiments where a reagent is supplied as a gas, an inert gaseous diluent can be used in the gas stream. In such embodiments, the inert atmosphere or inert gaseous diluent typically is an inert gas, meaning that the gas does not interact with the metathesis catalyst to impede catalysis to a substantial degree. For example, non-limiting examples of inert gases include helium, neon, argon, methane, and nitrogen, used individually or with each other and other inert gases.

The reactor design for the metathesis reaction can vary depending on a variety of factors, including, but not limited to, the scale of the reaction, the reaction conditions (heat, pressure, etc.), the identity of the catalyst, the identity of the materials being reacted in the reactor, and the nature of the feedstock being employed. Suitable reactors can be designed by those of skill in the art, depending on the relevant factors, and incorporated into a refining process such, such as those disclosed herein.

The metathesis reactions disclosed herein generally occur in the presence of one or more metathesis catalysts. Such methods can employ any suitable metathesis catalyst. The metathesis catalyst in this reaction may include any catalyst or catalyst system that catalyzes a metathesis reaction. Any known or future developed metathesis catalyst may be used, alone or in combination with one or more additional catalysts. Examples of metathesis catalysts and process conditions are described in US 2011/0160472, incorporated by reference herein in its entirety, except that in the event of any inconsistent disclosure or definition from the present specification, the disclosure or definition herein shall be deemed to prevail. A number of the metathesis catalysts described in US 2011/0160472 are presently available from Materia, Inc. (Pasadena, Calif.).

In some embodiments, the metathesis catalyst includes a Grubbs-type olefin metathesis catalyst and/or an entity derived therefrom. In some embodiments, the metathesis catalyst includes a first-generation Grubbs-type olefin metathesis catalyst and/or an entity derived therefrom. In some embodiments, the metathesis catalyst includes a second-generation Grubbs-type olefin metathesis catalyst and/or an entity derived therefrom. In some embodiments, the metathesis catalyst includes a first-generation Hoveyda-Grubbs-type olefin metathesis catalyst and/or an entity derived therefrom. In some embodiments, the metathesis catalyst includes a second-generation Hoveyda-Grubbs-type olefin metathesis catalyst and/or an entity derived therefrom. In some embodiments, the metathesis catalyst includes one or a plurality of the ruthenium carbene metathesis catalysts sold by Materia, Inc. of Pasadena, Calif. and/or one or more entities derived from such catalysts. Representative metathesis catalysts from Materia, Inc. for use in accordance with the present teachings include but are not limited to those sold under the following product numbers as well as combinations thereof: product no. C823 (CAS no. 172222-30-9), product no. C848 (CAS no. 246047-72-3), product no. C601 (CAS no. 203714-71-0), product no. C627 (CAS no. 301224-40-8), product no. C571 (CAS no. 927429-61-6), product no. C598 (CAS no. 802912-44-3), product no. C793 (CAS no. 927429-60-5), product no. C801 (CAS no. 194659-03-9), product no. C827 (CAS no. 253688-91-4), product no. C884 (CAS no. 900169-53-1), product no. C833 (CAS no. 1020085-61-3), product no. C859 (CAS no. 832146-68-6), product no. C711 (CAS no. 635679-24-2), product no. C933 (CAS no. 373640-75-6).

In some embodiments, the metathesis catalyst includes a molybdenum and/or tungsten carbene complex and/or an entity derived from such a complex. In some embodiments, the metathesis catalyst includes a Schrock-type olefin metathesis catalyst and/or an entity derived therefrom. In some embodiments, the metathesis catalyst includes a high-oxidation-state alkylidene complex of molybdenum and/or an entity derived therefrom. In some embodiments, the metathesis catalyst includes a high-oxidation-state alkylidene complex of tungsten and/or an entity derived therefrom. In some embodiments, the metathesis catalyst includes molybdenum (VI). In some embodiments, the metathesis catalyst includes tungsten (VI). In some embodiments, the metathesis catalyst includes a molybdenum- and/or a tungsten-containing alkylidene complex of a type described in one or more of (a) Angew. Chem. Int. Ed. Engl., 2003, 42, 4592-4633; (b) Chem. Rev., 2002, 102, 145-179; and/or (c) Chem. Rev., 2009, 109, 3211-3226, each of which is incorporated by reference herein in its entirety, except that in the event of any inconsistent disclosure or definition from the present specification, the disclosure or definition herein shall be deemed to prevail.

Suitable homogeneous metathesis catalysts include combinations of a transition metal halide or oxo-halide (e.g., WOCl₄ or WCl₆) with an alkylating cocatalyst (e.g., Me₄Sn), or alkylidene (or carbene) complexes of transition metals, particularly Ru or W. These include first and second-generation Grubbs catalysts, Grubbs-Hoveyda catalysts, and the like. Suitable alkylidene catalysts have the general structure: M[X¹X²L¹L²(L³)ₙ]=Cₘ═C(R¹)R²

where M is a Group 8 transition metal, L¹, L², and L³ are neutral electron donor ligands, n is 0 (such that L³ may not be present) or 1, m is 0, 1, or 2, X¹ and X² are anionic ligands, and R¹ and R² are independently selected from H, hydrocarbyl, substituted hydrocarbyl, heteroatom-containing hydrocarbyl, substituted heteroatom-containing hydrocarbyl, and functional groups. Any two or more of X¹, X², L¹, L², L³, R¹ and R² can form a cyclic group and any one of those groups can be attached to a support.

First-generation Grubbs catalysts fall into this category where m=n=0 and particular selections are made for n, X¹, X², L¹, L², L³, R¹ and R² as described in U.S. Pat. Appl. Publ. No. 2010/0145086, the teachings of which related to all metathesis catalysts are incorporated herein by reference.

Second-generation Grubbs catalysts also have the general formula described above, but L¹ is a carbene ligand where the carbene carbon is flanked by N, O, S, or P atoms, preferably by two N atoms. Usually, the carbene ligand is part of a cyclic group. Examples of suitable second-generation Grubbs catalysts also appear in the '086 publication.

In another class of suitable alkylidene catalysts, L¹ is a strongly coordinating neutral electron donor as in first- and second-generation Grubbs catalysts, and L² and L³ are weakly coordinating neutral electron donor ligands in the form of optionally substituted heterocyclic groups. Thus, L² and L³ are pyridine, pyrimidine, pyrrole, quinoline, thiophene, or the like.

In yet another class of suitable alkylidene catalysts, a pair of substituents is used to form a bi- or tridentate ligand, such as a biphosphine, dialkoxide, or alkyldiketonate. Grubbs-Hoveyda catalysts are a subset of this type of catalyst in which L² and R² are linked. Typically, a neutral oxygen or nitrogen coordinates to the metal while also being bonded to a carbon that is α-, β-, or γ- with respect to the carbene carbon to provide the bidentate ligand. Examples of suitable Grubbs-Hoveyda catalysts appear in the '086 publication.

The structures below provide just a few illustrations of suitable catalysts that may be used:

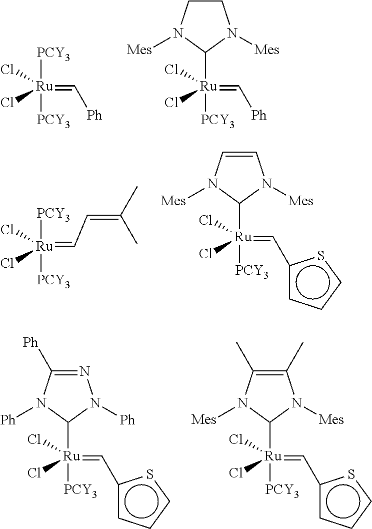

An immobilized catalyst can be used for the metathesis process. An immobilized catalyst is a system comprising a catalyst and a support, the catalyst associated with the support. Exemplary associations between the catalyst and the support may occur by way of chemical bonds or weak interactions (e.g. hydrogen bonds, donor acceptor interactions) between the catalyst, or any portions thereof, and the support or any portions thereof. Support is intended to include any material suitable to support the catalyst. Typically, immobilized catalysts are solid phase catalysts that act on liquid or gas phase reactants and products. Exemplary supports are polymers, silica or alumina. Such an immobilized catalyst may be used in a flow process. An immobilized catalyst can simplify purification of products and recovery of the catalyst so that recycling the catalyst may be more convenient.

Any useful amount of the selected metathesis catalyst can be used in the process. For example, the molar ratio of the unsaturated polyol ester to catalyst may range from about 5:1 to about 10,000,000:1 or from about 50:1 to 500,000:1. In some embodiments, an amount of about 1 to about 20 ppm, or about 2 ppm to about 15 ppm, of the metathesis catalyst per double bond of the starting composition (i.e., on a mole/mole basis) is used.

In some embodiments, the metathesis reaction is catalyzed by a system containing both a transition and a non-transition metal component. The most active and largest number of catalyst systems are derived from Group 6 and Group 8 transition metals, for example, tungsten, molybdenum, and ruthenium.

In certain embodiments, the metathesis catalyst is dissolved in a solvent prior to conducting the metathesis reaction. In certain such embodiments, the solvent chosen may be selected to be substantially inert with respect to the metathesis catalyst. For example, substantially inert solvents include, without limitation: aromatic hydrocarbons, such as benzene, toluene, xylenes, etc.; halogenated aromatic hydrocarbons, such as chlorobenzene and dichlorobenzene; aliphatic solvents, including pentane, hexane, heptane, cyclohexane, etc.; and chlorinated alkanes, such as dichloromethane, chloroform, dichloroethane, etc. In some embodiments, the solvent comprises toluene.

In other embodiments, the metathesis catalyst is not dissolved in a solvent prior to conducting the metathesis reaction. The catalyst, instead, for example, can be slurried with the natural oil or unsaturated ester, where the natural oil or unsaturated ester is in a liquid state. Under these conditions, it is possible to eliminate the solvent (e.g., toluene) from the process and eliminate downstream olefin losses when separating the solvent. In other embodiments, the metathesis catalyst may be added in solid state form (and not slurried) to the natural oil or unsaturated ester (e.g., as an auger feed).

In certain embodiments, a ligand may be added to the metathesis reaction mixture. In many embodiments using a ligand, the ligand is selected to be a molecule that stabilizes the catalyst, and may thus provide an increased turnover number for the catalyst. In some cases the ligand can alter reaction selectivity and product distribution. Examples of ligands that can be used include Lewis base ligands, such as, without limitation, trialkylphosphines, for example tricyclohexylphosphine and tributyl phosphine; triarylphosphines, such as triphenylphosphine; diarylalkylphosphines, such as, diphenylcyclohexylphosphine; pyridines, such as 2,6-dimethylpyridine, 2,4,6-trimethylpyridine; as well as other Lewis basic ligands, such as phosphine oxides and phosphinites. Additives may also be present during metathesis that increase catalyst lifetime.

The metathesis reaction temperature may, in some instances, be a rate-controlling variable where the temperature is selected to provide a desired product at an acceptable rate. In certain embodiments, the metathesis reaction temperature is greater than about −40° C., or greater than about −20° C., or greater than about 0° C., or greater than about 10° C. In certain embodiments, the metathesis reaction temperature is less than about 200° C., or less than about 150° C., or less than about 120° C. In some embodiments, the metathesis reaction temperature is between about 0° C. and about 150° C., or is between about 10° C. and about 120° C.

The metathesis reaction can be run under any desired pressure. Typically, it will be desirable to maintain a total pressure that is high enough to keep the cross-metathesis reagent in solution. Therefore, as the molecular weight of the cross-metathesis reagent increases, the lower pressure range typically decreases since the boiling point of the cross-metathesis reagent increases. The total pressure may be selected to be greater than about 0.1 atm (10 kPa), in some embodiments greater than about 0.3 atm (30 kPa), or greater than about 1 atm (100 kPa). Typically, the reaction pressure is no more than about 70 atm (7000 kPa), in some embodiments no more than about 30 atm (3000 kPa). A non-limiting exemplary pressure range for the metathesis reaction is from about 1 atm (100 kPa) to about 30 atm (3000 kPa). In certain embodiments it may be desirable to run the metathesis reactions under an atmosphere of reduced pressure. Conditions of reduced pressure or vacuum may be used to remove olefins as they are generated in a metathesis reaction, thereby driving the metathesis equilibrium towards the formation of less volatile products. In the case of a self-metathesis of a natural oil, reduced pressure can be used to remove C₁₂ or lighter olefins including, but not limited to, hexene, nonene, and dodecene, as well as byproducts including, but not limited to cyclohexadiene and benzene as the metathesis reaction proceeds. The removal of these species can be used as a means to drive the reaction towards the formation of diester groups and cross linked triglycerides.

In some embodiments, after metathesis has occurred, the metathesis catalyst is removed from the resulting product. One method of removing the catalyst is treatment of the metathesized product with an adsorbent bed. Representative adsorbents for use in accordance with the present teachings include but are not limited to carbon, silica, silica-alumina, alumina, clay, magnesium silicates (e.g., Magnesols), the synthetic silica adsorbent sold under the tradename TRISYL by W. R. Grace & Co., diatomaceous earth, polystyrene, macroporous (MP) resins, and the like, and combinations thereof. In one embodiment, the adsorbent is a clay bed. The clay bed will adsorb the metathesis catalyst, and after a filtration step, the metathesized product can be sent to a separation unit for further processing. The separation unit may comprise a distillation unit. In some embodiments, the distillation may be conducted, for example, by steam stripping the metathesized product. Distilling may be accomplished by sparging the mixture in a vessel, typically agitated, by contacting the mixture with a gaseous stream in a column that may contain typical distillation packing (e.g., random or structured), by vacuum distillation, or evaporating the lights in an evaporator such as a wiped film evaporator. Typically, steam stripping will be conducted at reduced pressure and at temperatures ranging from about 100° C. to 250° C. The temperature may depend, for example, on the level of vacuum used, with higher vacuum allowing for a lower temperature and allowing for a more efficient and complete separation of volatiles.

In another embodiment, the adsorbent is a water soluble phosphine reagent such as tris hydroxymethyl phosphine (THMP). THMP may be added at a rate equivalent to at least 1:1, 5:1, 10:1, 25:1, or 50:1 molar ratio relative to the catalyst. Catalyst may be separated with a water soluble phosphine through known liquid-liquid extraction mechanisms by decanting the aqueous phase from the organic phase. In other embodiments, the catalyst separation comprises washing or extracting the mixture with a polar solvent (e.g., particularly, though not exclusively, for embodiments in which the reagent is at least partially soluble in the polar solvent). Representative polar solvents for use in accordance with the present teachings include but are not limited to water, alcohols (e.g., methanol, ethanol, etc.), ethylene glycol, glycerol, DMF, multifunctional polar compounds including but not limited to polyethylene glycols and/or glymes, ionic liquids, and the like, and combinations thereof. In some embodiments, the mixture is extracted with water. In some embodiments, when a phosphite ester that is at least partially hydrolyzable (e.g., in some embodiments, a phosphite ester having a low molecular weight, including but not limited to trimethyl phosphite, triethyl phosphite, and a combination thereof) is used as a reagent, washing the mixture with water may convert the phosphite ester into a corresponding acid. In other embodiments, the metathesized product may be contacted with a reactant to deactivate or to extract the catalyst.

The metathesis reaction also results in the formation of internal olefin compounds that may be linear or cyclic. If the metathesized polyol ester is fully or partially hydrogenated, the linear and cyclic olefins would typically be fully or partially converted to the corresponding saturated linear and cyclic hydrocarbons. The linear/cyclic olefins and saturated linear/cyclic hydrocarbons may remain in the metathesized polyol ester or they may be removed or partially removed from the metathesized polyol ester using one or more known stripping techniques, including but not limited to wipe film evaporation, falling film evaporation, rotary evaporation, steam stripping, vacuum distillation, etc.

Multiple, sequential metathesis reaction steps may be employed. For example, the glyceride copolymer product may be made by reacting an unsaturated polyol ester in the presence of a metathesis catalyst to form a first glyceride copolymer product. The first glyceride copolymer product may then be reacted in a self-metathesis reaction to form another glyceride copolymer product. Alternatively, the first glyceride copolymer product may be reacted in a cross-metathesis reaction with an unsaturated polyol ester to form another glyceride copolymer product. Also in the alternative, the transesterified products, the olefins and/or esters may be further metathesized in the presence of a metathesis catalyst. Such multiple and/or sequential metathesis reactions can be performed as many times as needed, and at least one or more times, depending on the processing/compositional requirements as understood by a person skilled in the art. As used herein, a “glyceride copolymer product” may include products that have been once metathesized and/or multiply metathesized. These procedures may be used to form metathesis dimers, metathesis trimers, metathesis tetramers, metathesis pentamers, and higher order metathesis oligomers (e.g., metathesis hexamers, metathesis heptamers, metathesis octamers, metathesis nonamers, metathesis decamers, and higher than metathesis decamers). These procedures can be repeated as many times as desired (for example, from 2 to about 50 times, or from 2 to about 30 times, or from 2 to about 10 times, or from 2 to about 5 times, or from 2 to about 4 times, or 2 or 3 times) to provide the desired metathesis oligomer or polymer which may comprise, for example, from 2 to about 100 bonded groups, or from 2 to about 50, or from 2 to about 30, or from 2 to about 10, or from 2 to about 8, or from 2 to about 6 bonded groups, or from 2 to about 4 bonded groups, or from 2 to about 3 bonded groups. In certain embodiments, it may be desirable to use the glyceride copolymer products produced by cross metathesis of an unsaturated polyol ester, or blend of unsaturated polyol esters, with a C₂₋₁₄ olefin, preferably C₂₋₆ olefin, more preferably C₄ olefin, and mixtures and isomers thereof, as the reactant in a self-metathesis reaction to produce another glyceride copolymer product. Alternatively, metathesized products produced by cross metathesis of an unsaturated polyol ester, or blend of unsaturated polyol esters, with a C₂₋₁₄ olefin, preferably C₂₋₆ olefin, more preferably C₄ olefin, and mixtures and isomers thereof, can be combined with an unsaturated polyol ester, or blend of unsaturated polyol esters, and further metathesized to produce another glyceride copolymer product.

In some embodiments, the glyceride copolymer may be hydrogenated (e.g., fully or partially hydrogenated) in order to improve the stability of the oil or to modify its viscosity or other properties. Representative techniques for hydrogenating unsaturated polyol esters are known in the art and are discussed herein.

In other embodiments, the glyceride copolymers can be used as a blend with one or more hair care benefit agents and/or hair conditioning actives.

Hydrogenation:

In some embodiments, the unsaturated polyol ester is partially hydrogenated before it is subjected to the metathesis reaction. Partial hydrogenation of the unsaturated polyol ester reduces the number of double bonds that are available for in the subsequent metathesis reaction. In some embodiments, the unsaturated polyol ester is metathesized to form a glyceride copolymer, and the glyceride copolymer is then hydrogenated (e.g., partially or fully hydrogenated) to form a hydrogenated glyceride copolymer.

Hydrogenation may be conducted according to any known method for hydrogenating double bond-containing compounds such as vegetable oils. In some embodiments, the unsaturated polyol ester, natural oil or glyceride copolymer is hydrogenated in the presence of a nickel catalyst that has been chemically reduced with hydrogen to an active state. Commercial examples of supported nickel hydrogenation catalysts include those available under the trade designations “NYSOFACT”, “NYSOSEL”, and “NI 5248 D” (from Englehard Corporation, Iselin, N.H.). Additional supported nickel hydrogenation catalysts include those commercially available under the trade designations “PRICAT 9910”, “PRICAT 9920”, “PRICAT 9908”, “PRICAT 9936” (from Johnson Matthey Catalysts, Ward Hill, Mass.).

In some embodiments, the hydrogenation catalyst comprising, for example, nickel, copper, palladium, platinum, molybdenum, iron, ruthenium, osmium, rhodium, or iridium. Combinations of metals may also be used. Useful catalyst may be heterogeneous or homogeneous. In some embodiments, the catalysts are supported nickel or sponge nickel type catalysts.

In some embodiments, the hydrogenation catalyst comprises nickel that has been chemically reduced with hydrogen to an active state (i.e., reduced nickel) provided on a support. In some embodiments, the support comprises porous silica (e.g., kieselguhr, infusorial, diatomaceous, or siliceous earth) or alumina. The catalysts are characterized by a high nickel surface area per gram of nickel.

In some embodiments, the particles of supported nickel catalyst are dispersed in a protective medium comprising hardened triacylglyceride, edible oil, or tallow. In an exemplary embodiment, the supported nickel catalyst is dispersed in the protective medium at a level of about 22 wt. % nickel.

Hydrogenation may be carried out in a batch or in a continuous process and may be partial hydrogenation or complete hydrogenation. In a representative batch process, a vacuum is pulled on the headspace of a stirred reaction vessel and the reaction vessel is charged with the material to be hydrogenated (e.g., RBD soybean oil or metathesized RBD soybean oil). The material is then heated to a desired temperature. Typically, the temperature ranges from about 50° C. to 350° C., for example, about 100° C. to 300° C. or about 150° C. to 250° C. The desired temperature may vary, for example, with hydrogen gas pressure. Typically, a higher gas pressure will require a lower temperature. In a separate container, the hydrogenation catalyst is weighed into a mixing vessel and is slurried in a small amount of the material to be hydrogenated (e.g., RBD soybean oil or metathesized RBD soybean oil). When the material to be hydrogenated reaches the desired temperature, the slurry of hydrogenation catalyst is added to the reaction vessel. Hydrogen gas is then pumped into the reaction vessel to achieve a desired pressure of H₂ gas. Typically, the H₂ gas pressure ranges from about 15 to 3000 psig or, for example, about 15 psig to 150 psig. As the gas pressure increases, more specialized high-pressure processing equipment may be required. Under these conditions the hydrogenation reaction begins and the temperature is allowed to increase to the desired hydrogenation temperature (e.g., about 120° C. to 200° C.) where it is maintained by cooling the reaction mass, for example, with cooling coils. When the desired degree of hydrogenation is reached, the reaction mass is cooled to the desired filtration temperature.

The amount of hydrogenation catalysts is typically selected in view of a number of factors including, for example, the type of hydrogenation catalyst used, the amount of hydrogenation catalyst used, the degree of unsaturation in the material to be hydrogenated, the desired rate of hydrogenation, the desired degree of hydrogenation (e.g., as measure by iodine value (IV)), the purity of the reagent, and the H₂ gas pressure. In some embodiments, the hydrogenation catalyst is used in an amount of about 10 wt. % or less, for example, about 5 wt. % or less or about 1 wt. % or less.

After hydrogenation, the hydrogenation catalyst may be removed from the hydrogenated product using known techniques, for example, by filtration. In some embodiments, the hydrogenation catalyst is removed using a plate and frame filter such as those commercially available from Sparkler Filters, Inc., Conroe Tex. In some embodiments, the filtration is performed with the assistance of pressure or a vacuum. In order to improve filtering performance, a filter aid may be used. A filter aid may be added to the metathesized product directly or it may be applied to the filter. Representative examples of filtering aids include diatomaceous earth, silica, alumina, and carbon. Typically, the filtering aid is used in an amount of about 10 wt. % or less, for example, about 5 wt. % or less or about 1 wt. % or less. Other filtering techniques and filtering aids may also be employed to remove the used hydrogenation catalyst. In other embodiments the hydrogenation catalyst is removed using centrifugation followed by decantation of the product.

Potential Processing Aids and/or Impurities

Unsaturated polyol esters, particularly those derived or synthesized from natural sources, are known to those skilled in the art to contain a wide range of minor components and impurities. These may include tocopherols, carotenes, free fatty acids, free glycerin, sterols, glucosinolates, phospholipids, peroxides, aldehydes and other oxidation products, and the like. The impurities and reactions products present in a wide range of natural oils are described in “Bailey's Industrial Oil and Fat Products,” Fifth edition, Y. H. Hui, Ed., Wiley (1996) and references cited therein; “Lipid Analysis in Oil and Fats,” R. J. Hamilton, Ed., Chapman Hall (1998) and references cited therein; and “Flavor Chemistry of Fats and Oils,” D. B. Min and T. H. Smouse, Ed., American Oil Chemists Society (1985) and references cited therein.

It is understood by one skilled in the art that any of these methods of making the glyceride copolymers claimed and described in this specification may result in the presence of impurities in the final glyceride copolymer and in the compositions/consumer products claimed and described in this specification as a result of the use of the glyceride copolymers. These nonlimiting examples include metathesis catalysts including metals and ligands described herein; immobilized catalyst supports including silica or alumina; oil pretreatment agents including reducing agents, cation-inorganic base compositions and adsorbents; structures which result from oil thermal pretreatment; process aids including solvents such as aromatic hydrocarbons, halogenated aromatic hydrocarbons, aliphatic solvents, and chlorinated alkanes; aliphatic olefins including hexane, nonene, dodecene, and cyclohexadiene; catalyst kill agents and/or catalyst removal agents including adsorbents such as clay, carbon, silica, silica-alumina, alumina, clay, magnesium silicates, synthetic silica, diatomaceous earth, polystyrene, macroporous (MP) resins, or water soluble phosphine reagents such as tris hydroxymethyl phosphine (THMP); polar solvents including water, alcohols (e.g., methanol, ethanol, etc.), ethylene glycol, glycerol, DMF, multifunctional polar compounds including but not limited to polyethylene glycols and/or glymes, or ionic liquids; phosphite ester hydrolysis byproducts; hydrogenation catalysts, including metals and ligands described herein; immobilized hydrogenation catalyst supports including porous silica or alumina; adjuncts necessary to protect, activate and/or remove the hydrogenation catalyst; and/or water.

The glyceride copolymers claimed and described in this specification may contain the following processing aids and/or impurities:

TABLE 1
Potential Processing Aids and/or Impurities in Glyceride copolymers

| Processing aids and/or | Range | Preferred Range |
|---|---|---|
| impurities | (ppm by weight) | (ppm by weight) |
| Ruthenium | 0-100 | 0-30 |
| Phosphorus | 1-2000 | 2-100 |
| Chloride | 2-200 | 3-20 |

TABLE 2
Potential Processing Aids and/or Impurities in Consumer Products
Arising from Glyceride Copolymers
The following processing aids and/or impurities may be brought into
or generated during storage in the compositions/consumer products
claimed and described in this specification as a result of the use of
the glyceride copolymers, at the levels provided in this specification:

| | | | More Preferred |
|---|---|---|---|
| Processing aids | Range (ppm | Preferred Range | Range |
| and/or impurities | by weight) | (ppm by weight) | (ppm by weight) |
| Ruthenium (ppmwt) | 0-50 | 0-10 | 0-3 |
| Phosphorus (ppmwt) | 0.5-1000 | 0.1-200 | 0.2-10 |
| Chloride (ppmwt) | 1-100 | 0.2-20 | 0.3-2 |

### B. Cationic Surfactant System

The composition of the present invention comprises a cationic surfactant system. The cationic surfactant system can be one cationic surfactant or a mixture of two or more cationic surfactants. Preferably, the cationic surfactant system is selected from: mono-long alkyl quaternized ammonium salt; a combination of mono-long alkyl quaternized ammonium salt and di-long alkyl quaternized ammonium salt; mono-long alkyl amidoamine salt; a combination of mono-long alkyl amidoamine salt and di-long alkyl quaternized ammonium salt, a combination of mono-long alkyl amindoamine salt and mono-long alkyl quaternized ammonium salt.

The cationic surfactant system is included in the hair care composition at a level by weight of from about 0.1% to about 10%, preferably from about 0.5% to about 8%, more preferably from about 0.8% to about 5%, still more preferably from about 1.0% to about 4%.

Mono-Long Alkyl Quaternized Ammonium Salt

The monoalkyl quaternized ammonium salt cationic surfactants useful herein are those having one long alkyl chain which has from 12 to 30 carbon atoms, preferably from 16 to 24 carbon atoms, more preferably C18-22 alkyl group. The remaining groups attached to nitrogen are independently selected from an alkyl group of from 1 to about 4 carbon atoms or an alkoxy, polyoxyalkylene, alkylamido, hydroxyalkyl, aryl or alkylaryl group having up to about 4 carbon atoms.

Mono-long alkyl quaternized ammonium salts useful herein are those having the formula (I):

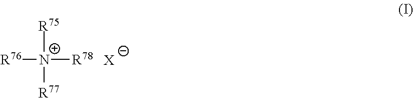

wherein one of R⁷⁵, R⁷⁶, R⁷⁷ and R⁷⁸ is selected from an alkyl group of from 12 to 30 carbon atoms or an aromatic, alkoxy, polyoxyalkylene, alkylamido, hydroxyalkyl, aryl or alkylaryl group having up to about 30 carbon atoms; the remainder of R⁷⁵, R⁷⁶, R⁷⁷ and R⁷⁸ are independently selected from an alkyl group of from 1 to about 4 carbon atoms or an alkoxy, polyoxyalkylene, alkylamido, hydroxyalkyl, aryl or alkylaryl group having up to about 4 carbon atoms; and X⁻ is a salt-forming anion such as those selected from halogen, (e.g. chloride, bromide), acetate, citrate, lactate, glycolate, phosphate, nitrate, sulfonate, sulfate, alkylsulfate, and alkyl sulfonate radicals. The alkyl groups can contain, in addition to carbon and hydrogen atoms, ether and/or ester linkages, and other groups such as amino groups. The longer chain alkyl groups, e.g., those of about 12 carbons, or higher, can be saturated or unsaturated. Preferably, one of R⁷⁵, R⁷⁶, R⁷⁷ and R⁷⁸ is selected from an alkyl group of from 12 to 30 carbon atoms, more preferably from 16 to 24 carbon atoms, still more preferably from 18 to 22 carbon atoms, even more preferably 22 carbon atoms; the remainder of R⁷⁵, R⁷⁶, R⁷⁷ and R⁷⁸ are independently selected from CH₃, C₂H₅, C₂H₄OH, and mixtures thereof; and X is selected from the group consisting of Cl, Br, CH₃OSO₃, C₂H₅OSO₃, and mixtures thereof.

Nonlimiting examples of such mono-long alkyl quaternized ammonium salt cationic surfactants include: behenyl trimethyl ammonium salt; stearyl trimethyl ammonium salt; cetyl trimethyl ammonium salt; and hydrogenated tallow alkyl trimethyl ammonium salt.

Mono-Long Alkyl Amidoamine Salt

Mono-long alkyl amines are also suitable as cationic surfactants. Primary, secondary, and tertiary fatty amines are useful. Particularly useful are tertiary amido amines having an alkyl group of from about 12 to about 22 carbons. Exemplary tertiary amido amines include: stearamidopropyldimethylamine, stearamidopropyldiethylamine, stearamidoethyldiethylamine, stearamidoethyldimethylamine, palmitamidopropyldimethylamine, palmitamidopropyldiethylamine, palmitamidoethyldiethylamine, palmitamidoethyldimethylamine, behenamidopropyldimethylamine, behenamidopropyldiethylamine, behenamidoethyldiethylamine, behenamidoethyldimethylamine, arachidamidopropyldimethylamine, arachidamidopropyldiethylamine, arachidamidoethyldiethylamine, arachidamidoethyldimethylamine, diethylaminoethylstearamide.

Useful amines in the present invention are disclosed in U.S. Pat. No. 4,275,055, Nachtigal, et al. These amines can also be used in combination with acids such as l-glutamic acid, lactic acid, hydrochloric acid, malic acid, succinic acid, acetic acid, fumaric acid, tartaric acid, citric acid, l-glutamic hydrochloride, maleic acid, and mixtures thereof; more preferably l-glutamic acid, lactic acid, citric acid. The amines herein are preferably partially neutralized with any of the acids at a molar ratio of the amine to the acid of from about 1:0.3 to about 1:2, more preferably from about 1:0.4 to about 1:1.

Di-Long Alkyl Quaternized Ammonium Salt

Di-long alkyl quaternized ammonium salt is preferably combined with a mono-long alkyl quaternized ammonium salt or mono-long alkyl amidoamine salt. It is believed that such combination can provide easy-to rinse feel, compared to single use of a monoalkyl quaternized ammonium salt or mono-long alkyl amidoamine salt. In such combination with a mono-long alkyl quaternized ammonium salt or mono-long alkyl amidoamine salt, the di-long alkyl quaternized ammonium salts are used at a level such that the wt % of the dialkyl quaternized ammonium salt in the cationic surfactant system is in the range of preferably from about 10% to about 50%, more preferably from about 30% to about 45%.

The dialkyl quaternized ammonium salt cationic surfactants useful herein are those having two long alkyl chains having 12-30 carbon atoms, preferably 16-24 carbon atoms, more preferably 18-22 carbon atoms. The remaining groups attached to nitrogen are independently selected from an alkyl group of from 1 to about 4 carbon atoms or an alkoxy, polyoxyalkylene, alkylamido, hydroxyalkyl, aryl or alkylaryl group having up to about 4 carbon atoms.

Di-long alkyl quaternized ammonium salts useful herein are those having the formula (II):

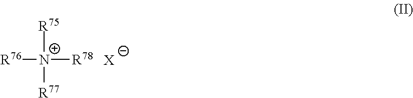

wherein two of R⁷⁵, R⁷⁶, R⁷⁷ and R⁷⁸ is selected from an alkyl group of from 12 to 30 carbon atoms or an aromatic, alkoxy, polyoxyalkylene, alkylamido, hydroxyalkyl, aryl or alkylaryl group having up to about 30 carbon atoms; the remainder of R⁷⁵, R⁷⁶, R⁷⁷ and R⁷⁸ are independently selected from an alkyl group of from 1 to about 4 carbon atoms or an alkoxy, polyoxyalkylene, alkylamido, hydroxyalkyl, aryl or alkylaryl group having up to about 4 carbon atoms; and X⁻ is a salt-forming anion such as those selected from halogen, (e.g. chloride, bromide), acetate, citrate, lactate, glycolate, phosphate, nitrate, sulfonate, sulfate, alkylsulfate, and alkyl sulfonate radicals. The alkyl groups can contain, in addition to carbon and hydrogen atoms, ether and/or ester linkages, and other groups such as amino groups. The longer chain alkyl groups, e.g., those of about 12 carbons, or higher, can be saturated or unsaturated. Preferably, one of R⁷⁵, R⁷⁶, R⁷⁷ and R⁷⁸ is selected from an alkyl group of from 12 to 30 carbon atoms, more preferably from 16 to 24 carbon atoms, still more preferably from 18 to 22 carbon atoms, even more preferably 22 carbon atoms; the remainder of R⁷⁵, R⁷⁶, R⁷⁷ and R⁷⁸ are independently selected from CH₃, C₂H₅, C₂H₄OH, and mixtures thereof; and X is selected from the group consisting of Cl, Br, CH₃OSO₃, C₂H₅OSO₃, and mixtures thereof.

Such dialkyl quaternized ammonium salt cationic surfactants include, for example, dialkyl (14-18) dimethyl ammonium chloride, ditallow alkyl dimethyl ammonium chloride, dihydrogenated tallow alkyl dimethyl ammonium chloride, distearyl dimethyl ammonium chloride, and dicetyl dimethyl ammonium chloride. Such dialkyl quaternized ammonium salt cationic surfactants also include, for example, asymmetric dialkyl quaternized ammonium salt cationic surfactants.

### C. High Melting Point Fatty Compound

The high melting point fatty compound useful herein have a melting point of 25° C. or higher, and is selected from the group consisting of fatty alcohols, fatty acids, fatty alcohol derivatives, fatty acid derivatives, and mixtures thereof. It is understood by the artisan that the compounds disclosed in this section of the specification can in some instances fall into more than one classification, e.g., some fatty alcohol derivatives can also be classified as fatty acid derivatives. However, a given classification is not intended to be a limitation on that particular compound, but is done so for convenience of classification and nomenclature. Further, it is understood by the artisan that, depending on the number and position of double bonds, and length and position of the branches, certain compounds having certain required carbon atoms may have a melting point of less than 25° C. Such compounds of low melting point are not intended to be included in this section. Nonlimiting examples of the high melting point compounds are found in International Cosmetic Ingredient Dictionary, Fifth Edition, 1993, and CTFA Cosmetic Ingredient Handbook, Second Edition, 1992.

Among a variety of high melting point fatty compounds, fatty alcohols are preferably used in the composition of the present invention. The fatty alcohols useful herein are those having from about 14 to about 30 carbon atoms, preferably from about 16 to about 22 carbon atoms. These fatty alcohols are saturated and can be straight or branched chain alcohols. Preferred fatty alcohols include, for example, cetyl alcohol, stearyl alcohol, behenyl alcohol, and mixtures thereof.

High melting point fatty compounds of a single compound of high purity are preferred. Single compounds of pure fatty alcohols selected from the group of pure cetyl alcohol, stearyl alcohol, and behenyl alcohol are highly preferred. By “pure” herein, what is meant is that the compound has a purity of at least about 90%, preferably at least about 95%. These single compounds of high purity provide good rinsability from the hair when the consumer rinses off the composition.

The high melting point fatty compound is included in the hair care composition at a level of from about 0.1% to about 20%, preferably from about 1% to about 15%, more preferably from about 1.5% to about 8% by weight of the composition, in view of providing improved conditioning benefits such as slippery feel during the application to wet hair, softness and moisturized feel on dry hair.

### D. Aqueous Carrier

The gel matrix of the hair care composition of the present invention includes an aqueous carrier. Accordingly, the formulations of the present invention can be in the form of pourable liquids (under ambient conditions). Such compositions will therefore typically comprise an aqueous carrier, which is present at a level of from about 20 wt % to about 95 wt %, or even from about 60 wt % to about 85 wt %. The aqueous carrier may comprise water, or a miscible mixture of water and organic solvent, and in one aspect may comprise water with minimal or no significant concentrations of organic solvent, except as otherwise incidentally incorporated into the composition as minor ingredients of other components.

The aqueous carrier useful in the present invention includes water and water solutions of lower alkyl alcohols and polyhydric alcohols. The lower alkyl alcohols useful herein are monohydric alcohols having 1 to 6 carbons, in one aspect, ethanol and isopropanol. The polyhydric alcohols useful herein include propylene glycol, hexylene glycol, glycerin, and propane diol.

According to embodiments of the present invention, the hair care compositions may have a pH in the range from about 2 to about 10, at 25° C. In one embodiment, the hair care composition has a pH in the range from about 2 to about 6, which may help to solubilize minerals and redox metals already deposited on the hair. Thus, the hair care composition can also be effective toward washing out the existing minerals and redox metals deposits, which can reduce cuticle distortion and thereby reduce cuticle chipping and damage.

### E. Gel Matrix

The composition of the present invention comprises a gel matrix. The gel matrix comprises a cationic surfactant, a high melting point fatty compound, and an aqueous carrier. The gel matrix is suitable for providing various conditioning benefits such as slippery feel during the application to wet hair and softness and moisturized feel on dry hair. In view of providing the above gel matrix, the cationic surfactant and the high melting point fatty compound are contained at a level such that the weight ratio of the cationic surfactant to the high melting point fatty compound is in the range of, preferably from about 1:1 to about 1:10, more preferably from about 1:1 to about 1:6.

### F. Additional Components

1. Silicone Conditioning Agent

According to embodiments of the present invention, the hair care composition includes a silicone conditioning agent which comprises a silicone compound. The silicone compound may comprise volatile silicone, non-volatile silicones, or combinations thereof. In one aspect, non-volatile silicones are employed. If volatile silicones are present, it will typically be incidental to their use as a solvent or carrier for commercially available forms of non-volatile silicone materials ingredients, such as silicone gums and resins. The silicone compounds may comprise a silicone fluid conditioning agent and may also comprise other ingredients, such as a silicone resin to improve silicone fluid deposition efficiency or enhance glossiness of the hair. The concentration of the silicone compound in the conditioner composition typically ranges from about 0.01 wt % to about 10 wt %, from about 0.1 wt % to about 8 wt %, from about 0.1 wt % to about 5 wt %, or even from about 0.2 wt % to about 3 wt %, for example

Exemplary silicone compounds include (a) a first polysiloxane which is non-volatile, substantially free of amino groups, and has a viscosity of from about 100,000 mm²s⁻¹ to about 30,000,000 mm²s⁻¹; (b) a second polysiloxane which is non-volatile, substantially free of amino groups, and has a viscosity of from about 5 mm²s⁻¹ to about 10,000 mm²s⁻¹; (c) an aminosilicone having less than about 0.5 wt % nitrogen by weight of the aminosilicone; (d) a silicone copolymer emulsion with an internal phase viscosity of greater than about 100×10⁶ mm²s⁻¹, as measured at 25° C.; (e) a silicone polymer containing quaternary groups; or (f) a grafted silicone polyol, wherein the silicone compounds (a)-(f) are disclosed in U.S. Patent Application Publication Nos. 2008/0292574, 2007/0041929, 2008/0292575, and 2007/0286837, each of which is incorporated by reference herein in its entirety.

a. First Polysiloxane

The hair care composition of the present invention may comprise a first polysiloxane. The first polysiloxane is non-volatile, and substantially free of amino groups. In the present invention, the first polysiloxanes being “substantially free of amino groups” means that the first polysiloxane contains 0 wt % of amino groups. The first polysiloxane has a viscosity of from about 100,000 mm²s⁻¹ to about 30,000,000 mm²s⁻¹ at 25° C. For example, the viscosity may range from about 300,000 mm²s⁻¹ to about 25,000,000 mm²s⁻¹, or from about 10,000,000 mm²s⁻¹ to about 20,000,000 mm²s⁻¹. The first polysiloxane has a molecular weight from about 100,000 to about 1,000,000. For example, the molecular weight may range from about 130,000 to about 800,000, or from about 230,000 to about 600,000. According to one aspect, the first polysiloxane may be nonionic.

Exemplary first non-volatile polysiloxanes useful herein include those in accordance with the following the general formula (I):

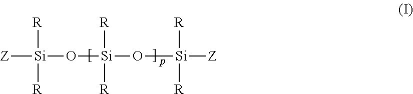

wherein R is alkyl or aryl, and p is an integer from about 1,300 to about 15,000, such as from about 1,700 to about 11,000, or from about 3,000 to about 8,000. Z represents groups which block the ends of the silicone chains. The alkyl or aryl groups substituted on the siloxane chain (R) or at the ends of the siloxane chains Z can have any structure as long as the resulting silicone remains fluid at room temperature, is dispersible, is neither irritating, toxic nor otherwise harmful when applied to the hair, is compatible with the other components of the composition, is chemically stable under normal use and storage conditions, and is capable of being deposited on and conditions the hair. According to an embodiment, suitable Z groups include hydroxy, methyl, methoxy, ethoxy, propoxy, and aryloxy. The two R groups on each silicon atom may represent the same group or different groups. According to one embodiment, the two R groups may represent the same group. Suitable R groups include methyl, ethyl, propyl, phenyl, methylphenyl and phenylmethyl. Exemplary silicone compounds include polydimethylsiloxane, polydiethylsiloxane, and polymethylphenylsiloxane. According to one embodiment, polydimethylsiloxane is the first polysiloxane. Commercially available silicone compounds useful herein include, for example, those available from the General Electric Company in their TSF451 series, and those available from Dow Corning in their Dow Corning SH200 series.

The silicone compounds that can be used herein also include a silicone gum. The term “silicone gum”, as used herein, means a polyorganosiloxane material having a viscosity at 25° C. of greater than or equal to 1,000,000 mm²s⁻¹. It is recognized that the silicone gums described herein can also have some overlap with the above-disclosed silicone compounds. This overlap is not intended as a limitation on any of these materials. The “silicone gums” will typically have a mass molecular weight in excess of about 165,000, generally between about 165,000 and about 1,000,000. Specific examples include polydimethylsiloxane, poly(dimethylsiloxane methylvinylsiloxane) copolymer, poly(dimethylsiloxane diphenylsiloxane methylvinylsiloxane) copolymer and mixtures thereof. Commercially available silicone gums useful herein include, for example, TSE200A available from the General Electric Company.

b. Second Polysiloxane

The hair care composition of the present invention may comprise a second polysiloxane. The second polysiloxane is non-volatile, and substantially free of amino groups. In the present invention, the second polysiloxane being “substantially free of amino groups” means that the second polysiloxane contains 0 wt % of amino groups. The second polysiloxane has a viscosity of from about 5 mm²s⁻¹ to about 10,000 mm²s⁻¹ at 25° C., such as from about 5 mm²s⁻¹ to about 5,000 mm²s⁻¹, from about 10 mm²s⁻¹ to about 1,000 mm²s⁻¹, or from about 20 mm²s⁻¹ to about 350 mm²s⁻¹. The second polysiloxane has a molecular weight of from about 400 to about 65,000. For example, the molecular weight of the second polysiloxane may range from about 800 to about 50,000, from about 400 to about 30,000, or from about 400 to about 15,000. According to one aspect, the second polysiloxane may be nonionic. According to another aspect, the second polysiloxane may be a linear silicone.

Exemplary second non-volatile polysiloxanes useful herein include polyalkyl or polyaryl siloxanes in accordance with the following the general formula (II):

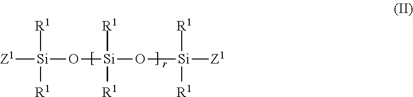

wherein R¹ is alkyl or aryl, and r is an integer from about 7 to about 850, such as from about 7 to about 665, from about 7 to about 400, or from about 7 to about 200. Z¹ represents groups which block the ends of the silicone chains. The alkyl or aryl groups substituted on the siloxane chain (R¹) or at the ends of the siloxane chains Z¹ can have any structure as long as the resulting silicone remains fluid at room temperature, is dispersible, is neither irritating, toxic nor otherwise harmful when applied to the hair, is compatible with the other components of the composition, is chemically stable under normal use and storage conditions, and is capable of being deposited on and conditions the hair. According to an embodiment, suitable Z¹ groups include hydroxy, methyl, methoxy, ethoxy, propoxy, and aryloxy. The two R¹ groups on each silicon atom may represent the same group or different groups. According to one embodiment, the two R¹ groups may represent the same group. Suitable R¹ groups include methyl, ethyl, propyl, phenyl, methylphenyl and phenylmethyl. Exemplary silicone compounds include polydimethylsiloxane, polydiethylsiloxane, and polymethylphenylsiloxane. According to one embodiment, polydimethylsiloxane is the second polysiloxane. Commercially available silicone compounds useful herein include, for example, those available from the General Electric Company in their TSF451 series, and those available from Dow Corning in their Dow Corning SH200 series.

c. Aminosilicone

The hair care composition of the present invention may comprise an amino silicone having less than about 0.5 wt % nitrogen by weight of the aminosilicone, such as less than about 0.2 wt %, or less than about 0.1 wt %, in view of friction reduction benefit. It has been surprisingly found that higher levels of nitrogen (amine functional groups) in the amino silicone tend to result in less friction reduction, and consequently less conditioning benefit from the aminosilicone. The aminosilicone useful herein may have at least one silicone block with greater than 200 siloxane units, in view of friction reduction benefit. The aminosilicones useful herein include, for example, quaternized aminosilicone and non-quaternized aminosilicone.

In one embodiment, the aminosilicones useful herein are water-insoluble. In the present invention, “water-insoluble aminosilicone” means that the aminosilicone has a solubility of 10 g or less per 100 g water at 25° C., in another embodiment 5 g or less per 100 g water at 25° C., and in another embodiment 1 g or less per 100 g water at 25° C. In the present invention, “water-insoluble aminosilicone” means that the aminosilicone is substantially free of copolyol groups. If copolyol groups are present, they are present at a level of less than 10 wt %, less than 1 wt %, or less than 0.1 wt % by weight of the amionosilicone.

According to one embodiment, aminosilicone useful herein are those which conform to the general formula (III):

(R²)ₐG₃₋ₐ-Si(—O—SiG₂)ₙ(—O—SiG_{b}(R²)_{2-b})ₘ—O—SiG₃₋ₐ(R²)ₐ (IIII)

wherein G is hydrogen, phenyl, hydroxy, or C₁-C₈ alkyl, such as methyl; a is an integer having a value from 1 to 3, such as 1; b is an integer having a value from 0 to 2, such as 1; n is a number from 1 to 2,000, such as from 100 to 1,800, from 300 to 800, or from 500 to 600; m is an integer having a value from 0 to 1,999, such as from 0 to 10, or 0; R² is a monovalent radical conforming to the general formula C_{q}H_{2q}L, wherein q is an integer having a value from 2 to 8 and L is selected from the following groups: —N(R³₂)CH₂—CH₂—N(R³₂)₂; —N(R³)₂; —N⁺(R³)₃A⁻; —N(R³)CH₂—CH₂—N⁺R³H₂A⁻; wherein R³ is hydrogen, phenyl, benzyl, or a saturated hydrocarbon radical, such as an alkyl radical from about C₁ to about C₂₀; A⁻ is a halide ion. According to an embodiment, L is —N(CH₃)₂ or —NH₂. According to another embodiment, L is —NH₂.

The aminosilicone of the above formula is used at levels by weight of the composition of from about 0.1 wt % to about 5 wt %, alternatively from about 0.2 wt % to about 2 wt %, alternatively from about 0.2 wt % to about 1.0 wt %, and alternatively from about 0.3 wt % to about 0.8 wt %.

According to one embodiment, the aminosilicone may include those compounds corresponding to formula (III) wherein m=0; a=1; q=3; G=methyl; n is from about 1400 to about 1700, such as about 1600; and L is —N(CH₃)₂ or —NH₂, such as —NH₂. According to another embodiment, the aminosilicone may include those compounds corresponding to formula (III) wherein m=0; a=1; q=3; G=methyl; n is from about 400 to about 800, such as from about 500 to around 600; and L is L is —N(CH₃)₂ or —NH₂, such as —NH₂. Accordingly, the aforementioned aminosilicones can be called terminal aminosilicones, as one or both ends of the silicone chain are terminated by nitrogen containing group. Such terminal aminosilicones may provide improved friction reduction compared to graft aminosilicones.

Another example of an aminosilicone useful herein includes, for example, quaternized aminosilicone having a tradename KF8020 available from Shinetsu.

The above aminosilicones, when incorporated into the hair care composition, can be mixed with solvent having a lower viscosity. Such solvents include, for example, polar or non-polar, volatile or non-volatile oils. Such oils include, for example, silicone oils, hydrocarbons, and esters. Among such a variety of solvents, exemplary solvents include those selected from the group consisting of non-polar, volatile hydrocarbons, volatile cyclic silicones, non-volatile linear silicones, and mixtures thereof. The non-volatile linear silicones useful herein are those having a viscosity of from about 1 mm²s⁻¹ to about 20,000 mm²s⁻¹, such as from about 20 mm²s⁻¹ to about 10,000 mm²s⁻¹, at 25° C. According to one embodiment, the solvents are non-polar, volatile hydrocarbons, especially non-polar, volatile isoparaffins, in view of reducing the viscosity of the aminosilicones and providing improved hair conditioning benefits such as reduced friction on dry hair. Such mixtures may have a viscosity of from about 1,000 mPas to about 100,000 mPas, and alternatively from about 5,000 mPas to about 50,000 mPas.

d. Silicone Copolymer Emulsion

The hair care composition of the present invention may comprise a silicone copolymer emulsion with an internal phase viscosity of greater than about 100×10⁶ mm²s⁻¹. The silicone copolymer emulsion may be present in an amount of from about 0.1 wt % to about 15 wt %, alternatively from about 0.3 wt % to about 10 wt %, and alternatively about 0.5 wt % to about 5 wt %, by weight of the composition, in view of providing clean feel.

According to one embodiment, the silicone copolymer emulsion has a viscosity at 25° C. of greater than about 100×10⁶ mm²s⁻¹, alternatively greater than about 120×10⁶ mm²s⁻¹, and alternatively greater than about 150×10⁶ mm²s⁻¹. According to another embodiment, the silicone copolymer emulsion has a viscosity at 25° C. of less than about 1000×10⁶ mm²s⁻¹, alternatively less than about 500×10⁶ mm²s⁻¹, and alternatively less than about 300×10⁶ mm²s⁻¹. To measure the internal phase viscosity of the silicone copolymer emulsion, one may first break the polymer from the emulsion. By way of example, the following procedure can be used to break the polymer from the emulsion: 1) add 10 grams of an emulsion sample to 15 milliliters of isopropyl alcohol; 2) mix well with a spatula; 3) decant the isopropyl alcohol; 4) add 10 milliliters of acetone and knead polymer with spatula; 5) decant the acetone; 6) place polymer in an aluminum container and flatten/dry with a paper towel; and 7) dry for two hours in an 80° C. The polymer can then be tested using any known rheometer, such as, for example, a CarriMed, Haake, or Monsanto rheometer, which operates in the dynamic shear mode. The internal phase viscosity values can be obtained by recording the dynamic viscosity (n′) at a 9.900*10⁻³ Hz frequency point. According to one embodiment, the average particle size of the emulsions is less than about 1 micron, such as less than about 0.7 micron.

The silicone copolymer emulsions of the present invention may comprise a silicone copolymer, at least one surfactant, and water.

The silicone copolymer results from the addition reaction of the following two materials in the presence of a metal containing catalyst:

(i) a polysiloxane with reactive groups on both termini, represented by a general formula (IV):

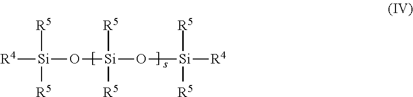

wherein:

R⁴ is a group capable of reacting by chain addition reaction such as, for example, a hydrogen atom, an aliphatic group with ethylenic unsaturation (i.e., vinyl, allyl, or hexenyl), a hydroxyl group, an alkoxyl group (i.e., methoxy, ethoxy, or propoxy), an acetoxyl group, or an amino or alkylamino group;

R⁵ is alkyl, cycloalkyl, aryl, or alkylaryl and may include additional functional groups such as ethers, hydroxyls, amines, carboxyls, thiols esters, and sulfonates; in an embodiment, R⁵ is methyl. Optionally, a small mole percentage of the groups may be reactive groups as described above for R⁵, to produce a polymer which is substantially linear but with a small amount of branching. In this case, the level of R⁵ groups equivalent to R⁴ groups may be less than about 10% on a mole percentage basis, such as less than about 2%;

s is an integer having a value such that the polysiloxane of formula (IV) has a viscosity of from about 1 mm²s⁻¹ to about 1×10⁶ mm²s⁻¹; and

(ii) at least one silicone compound or non-silicone compound comprising at least one or at most two groups capable of reacting with the R⁴ groups of the polysiloxane in formula (IV). According to one embodiment, the reactive group is an aliphatic group with ethylenic unsaturation.

The metal containing catalysts used in the above described reactions are often specific to the particular reaction. Such catalysts are known in the art. Generally, they are materials containing metals such as platinum, rhodium, tin, titanium, copper, lead, etc.

The mixture used to form the emulsion also may contain at least one surfactant. This can include non-ionic surfactants, cationic surfactants, anionic surfactants, alkylpolysaccharides, amphoteric surfactants, and the like. The above surfactants can be used individually or in combination.

An exemplary method of making the silicone copolymer emulsions described herein comprises the steps of 1) mixing materials (a) described above with material (b) described above, followed by mixing in an appropriate metal containing catalyst, such that material (b) is capable of reacting with material (a) in the presence of the metal containing catalyst; 2) further mixing in at least one surfactant and water; and 3) emulsifying the mixture. Methods of making such silicone copolymer emulsions are disclosed in U.S. Pat. No. 6,013,682; PCT Application No. WO 01/58986 A1; and European Patent Application No. EP0874017 A2.

A commercially available example of a silicone copolymer emulsion is an emulsion of about 60-70 wt % of divinyldimethicone/dimethicone copolymer having an internal phase viscosity of minimum 120×10⁶ mm²s⁻¹, available from Dow Corning with a tradename HMW2220.

e. Silicone Polymer Containing Quaternary Groups

The hair care composition of the present invention may comprise a silicone polymer containing quaternary groups (i.e., a quaternized silicone polymer). The quaternized silicone polymer provides improved conditioning benefits such as smooth feel, reduced friction, prevention of hair damage. Especially, the quaternary group can have good affinity with damaged/colorant hairs. The quaternized silicone polymer is present in an amount of from about 0.1 wt % to about 15 wt %, based on the total weight of the hair conditioning composition. For example, according to an embodiment, the quaternized silicone polymer may be present in an amount from about 0.2 wt % to about 10 wt %, alternatively from about 0.3 wt % to about 5 wt %, and alternatively from about 0.5 wt % to about 4 wt %, by weight of the composition.

The quaternized silicone polymer of the present invention is comprised of at least one silicone block and at least one non-silicone block containing quaternary nitrogen groups, wherein the number of the non-silicone blocks is one greater than the number of the silicone blocks. The silicone polymers correspond to the general structure (V):

A¹-B-(A²-B)ₘ-A¹ (V)

wherein, B is a silicone block having greater than 200 siloxane units; A¹ is an end group which may contain quaternary groups; A² is a non-silicone blocks containing quaternary nitrogen groups; and m is an integer 0 or greater, with the proviso that if m=0 then the A¹ group contains quaternary groups.

Structures corresponding to the general formula, for example, are disclosed in U.S. Pat. No. 4,833,225, in U.S. Patent Application Publication No. 2004/0138400, in U.S. Patent Application Publication No. 2004/0048996, and in U.S. Patent Application Publication No. 2008/0292575.

In one embodiment, the silicone polymers can be represented by the following structure (VI)

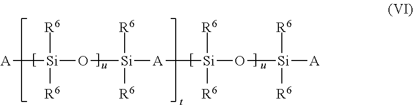

wherein, A is a group which contains at least one quaternary nitrogen group, and which is linked to the silicon atoms of the silicone block by a silicon-carbon bond, each A independently can be the same or different; R⁶ is an alkyl group of from about 1 to about 22 carbon atoms or an aryl group; each R⁶ independently can be the same or different; t is an integer having a value of from 0 or greater, for example t can be less than 20, or less than 10; and u is an integer greater than about 200, such as greater than about 250, or greater than about 300, and u may be less than about 700, or less than about 500. According to an embodiment, R⁶ is methyl.

f. Grafted Silicone Copolyol

The hair care composition of the present invention may comprise a grafted silicone copolyol in combination with the quaternized silicone polymer. It is believed that this grafted silicone copolyol can improve the spreadability of the quaternized silicone polymer by reducing the viscosity of the quaternized silicone polymer, and also can stabilize the quaternized silicone polymer in aqueous conditioner matrix. It is also believed that, by such improved spreadability, the hair care compositions of the present invention can provide better dry conditioning benefits such as friction reduction and/or prevention of damage with reduced tacky feel. It has been surprisingly found that the combination of the quaternized silicone polymer, grafted silicone copolyol, and cationic surfactant system comprising di-alkyl quaternized ammonium salt cationic surfactants provides improved friction reduction benefit, compared to a similar combination. Such similar combinations are, for example, a combination in which the grafted silicone copolyol is replaced with end-capped silicone copolyol, and another combination in which the cationic surfactant system is substantially free of di-alkyl quaternized ammonium salt cationic surfactants.

The grafted silicone copolyol is contained in the composition at a level such that the weight % of the grafted silicone copolyol to its mixture with quaternized silicone copolymer is in the range of from about 1 wt % to about 50 wt %, alternatively from about 5 wt % to about 40 wt %, and alternatively from about 10 wt % to 30 wt %.

The grafted silicone copolyols useful herein are those having a silicone backbone such as dimethicone backbone and polyoxyalkylene substitutions such as polyethylene oxide or/and polypropylene oxide substitutions. The grafted silicone copolyols useful herein have a hydrophilic-lipophilic balance (HLB) value of from about 5 to about 17, such as from about 8 to about 17, or from about 8 to about 12. The grafted silicone copolyols having the same INCI name have a variety of the weight ratio, depending on the molecular weight of the silicone portion and the number of the polyethylene oxide or/and polypropylene oxide substitutions.

According to an embodiment, exemplary commercially available grafted dimethicone copolyols include, for example: those having a tradename Silsoft 430 having an HLB value of from about 9 to about 12 (INCI name “PEG/PPG-20/23 dimethicone”) available from GE; those having a tradename Silsoft 475 having an HLB value of from about 13 to about 17 (INCI name “PEG-23/PPG-6 dimethicone”); those having a tradename Silsoft 880 having an HLB value of from about 13 to about 17 (INCI name “PEG-12 dimethicone”); those having a tradename Silsoft 440 having an HLB value of from about 9 to about 12 (INCI name “PEG-20/PPG-23 dimethicone”); those having a tradename DC5330 (INCI name “PEG-15/PPG-15 dimethicone”) available from Dow Corning.

The above quaternized silicone polymer and the grafted silicone copolyol may be mixed and emulsified by a emulsifying surfactant, prior to incorporating them into a gel matrix formed by cationic surfactants and high melting point fatty compounds, as discussed below. It is believed that, this pre-mixture can improve behavior of the quaternized silicone polymer and the grafted silicone copolyol, for example, increase the stability and reduce the viscosity to form more homogenized formulation together with the other components. Such emulsifying surfactant can be used at a level of about 0.001 wt % to about 1.5 wt %, alternatively from about 0.005% to about 1.0%, and alternatively from about 0.01 wt % to about 0.5 wt %, based on the total weight of the hair conditioning composition. Such surfactants may be nonionic, and have an HLB value of from about 2 to about 15, such as from about 3 to about 14, or from about 3 to about 10. Commercially available examples of emulsifying surfactant include nonionic surfactants having an INCI name C12-C14 Pareth-3 and having an HLB value of about 8 supplied from NIKKO Chemicals Co., Ltd. with tradename NIKKOL BT-3.

According to one embodiment, the hair care composition comprises a combination of two or more silicone conditioning agents, along with an EDDS sequestering agent and a gel matrix.

In one embodiment, the hair care composition comprises a polyalkylsiloxane mixture comprising (i) a first polyalkylsiloxane which is non-volatile, substantially free of amino groups, and has a viscosity of from about 100,000 mm²s⁻¹ to about 30,000,000 mm²s⁻¹, and (ii) a second polyalkylsiloxane which is non-volatile, substantially free of amino groups, and has a viscosity of from about 5 mm²s⁻¹ to about 10,000 mm²s⁻¹; an aminosilicone having less than about 0.5 wt % nitrogen by weight of the aminosilicone; and a silicone copolymer emulsion with an internal phase viscosity of greater than about 100×10⁶ mm²s⁻¹, as measured at 25° C. For example, in another embodiment, the hair care composition comprises from about 0.5 wt % to about 10 wt % of a polyalkylsiloxane mixture comprising (i) a first polyalkylsiloxane which is non-volatile, substantially free of amino groups, and has a viscosity of from about 100,000 mm²s⁻¹ to about 30,000,000 mm²s⁻¹, and (ii) a second polyalkylsiloxane which is non-volatile, substantially free of amino groups, and has a viscosity of from about 5 mm²s⁻¹ to about 10,000 mm²s⁻¹; from about 0.1 wt % to about 5 wt % of an aminosilicone having less than about 0.5 wt % nitrogen by weight of the aminosilicone; and from about 0.1 wt % to about 5 wt % of a silicone copolymer emulsion with an internal phase viscosity of greater than about 100×10⁶ mm²s⁻¹, as measured at 25° C.

In another embodiment, the hair care composition comprises a silicone polymer containing quaternary groups wherein said silicone polymer comprises silicone blocks with greater than about 200 siloxane units; and a grafted silicone copolyol. For example, in another embodiment, the hair care composition comprises from about 0.1 wt % to about 15 wt % of a silicone polymer containing quaternary groups wherein said silicone polymer comprises silicone blocks with greater than about 200 siloxane units; and a grafted silicone copolyol at a level such that the weight % of the grafted silicone copolyol in its mixture with the quaternized silicone polymer is in the range of from about 1 wt % to about 50 wt %.

In yet another embodiment, the hair care composition comprises an aminosilicone having a viscosity of from about 1,000 centistokes to about 1,000,000 centistokes, and less than about 0.5% nitrogen by weight of the aminosilicone; and (2) a silicone copolymer emulsion with an internal phase viscosity of greater than about 120×10⁶ centistokes, as measured at 25° C.

2. Other Conditioning Agents

Also suitable for use in the hair care compositions herein are the conditioning agents described by the Procter & Gamble Company in U.S. Pat. Nos. 5,674,478, and 5,750,122. Also suitable for use herein are those conditioning agents described in U.S. Pat. Nos. 4,529,586, 4,507,280, 4,663,158, 4,197,865, 4,217, 914, 4,381,919, and 4,422, 853.

a. Organic Conditioning Oils

The hair care compositions of the present invention may also further comprise an organic conditioning oil. According to embodiments of the present invention, the hair care composition may comprise from about 0.05 wt % to about 3 wt %, from about 0.08 wt % to about 1.5 wt %, or even from about 0.1 wt % to about 1 wt %, of at least one organic conditioning oil as the conditioning agent, in combination with other conditioning agents, such as the silicones (described herein). Suitable conditioning oils include hydrocarbon oils, polyolefins, and fatty esters. Suitable hydrocarbon oils include, but are not limited to, hydrocarbon oils having at least about 10 carbon atoms, such as cyclic hydrocarbons, straight chain aliphatic hydrocarbons (saturated or unsaturated), and branched chain aliphatic hydrocarbons (saturated or unsaturated), including polymers and mixtures thereof. Straight chain hydrocarbon oils are typically from about C12 to about C19. Branched chain hydrocarbon oils, including hydrocarbon polymers, typically will contain more than 19 carbon atoms. Suitable polyolefins include liquid polyolefins, liquid poly-α-olefins, or even hydrogenated liquid poly-α-olefins. Polyolefins for use herein may be prepared by polymerization of C4 to about C14 or even C6 to about C12. Suitable fatty esters include, but are not limited to, fatty esters having at least 10 carbon atoms. These fatty esters include esters with hydrocarbyl chains derived from fatty acids or alcohols (e.g. mono-esters, polyhydric alcohol esters, and di- and tri-carboxylic acid esters). The hydrocarbyl radicals of the fatty esters hereof may include or have covalently bonded thereto other compatible functionalities, such as amides and alkoxy moieties (e.g., ethoxy or ether linkages, etc.).

3. Nonionic Polymers

The hair care composition of the present invention may also further comprise a nonionic polymer. According to an embodiment, the conditioning agent for use in the hair care composition of the present invention may include a polyalkylene glycol polymer. For example, polyalkylene glycols having a molecular weight of more than about 1000 are useful herein. Useful are those having the following general formula (VIII):

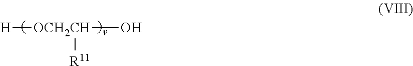

wherein R¹¹ is selected from the group consisting of H, methyl, and mixtures thereof; and v is the number of ethoxy units. The polyalkylene glycols, such as polyethylene glycols, can be included in the hair care compositions of the present invention at a level of from about 0.001 wt % to about 10 wt %. In an embodiment, the polyethylene glycol is present in an amount up to about 5 wt % based on the weight of the composition. Polyethylene glycol polymers useful herein are PEG-2M (also known as Polyox WSR® N-10, which is available from Union Carbide and as PEG-2,000); PEG-5M (also known as Polyox WSR® N-35 and Polyox WSR® N-80, available from Union Carbide and as PEG-5,000 and Polyethylene Glycol 300,000); PEG-7M (also known as Polyox WSR® N-750 available from Union Carbide); PEG-9M (also known as Polyox WSR® N-3333 available from Union Carbide); and PEG-14 M (also known as Polyox WSR® N-3000 available from Union Carbide).

4. Suspending Agent

The hair care compositions of the present invention may further comprise a suspending agent at concentrations effective for suspending water-insoluble material in dispersed form in the compositions or for modifying the viscosity of the composition. Such concentrations range from about 0.1 wt % to about 10 wt %, or even from about 0.3 wt % to about 5.0 wt %.

Suspending agents useful herein include anionic polymers and nonionic polymers. Useful herein are vinyl polymers such as cross linked acrylic acid polymers with the CTFA name Carbomer, cellulose derivatives and modified cellulose polymers such as methyl cellulose, ethyl cellulose, hydroxyethyl cellulose, hydroxypropyl methyl cellulose, nitro cellulose, sodium cellulose sulfate, sodium carboxymethyl cellulose, crystalline cellulose, cellulose powder, polyvinylpyrrolidone, polyvinyl alcohol, guar gum, hydroxypropyl guar gum, xanthan gum, arabia gum, tragacanth, galactan, carob gum, guar gum, karaya gum, carrageenan, pectin, agar, quince seed (Cydonia oblonga Mill), starch (rice, corn, potato, wheat), algae colloids (algae extract), microbiological polymers such as dextran, succinoglucan, pulleran, starch-based polymers such as carboxymethyl starch, methylhydroxypropyl starch, alginic acid-based polymers such as sodium alginate, alginic acid propylene glycol esters, acrylate polymers such as sodium polyacrylate, polyethylacrylate, polyacrylamide, polyethyleneimine, and inorganic water soluble material such as bentonite, aluminum magnesium silicate, laponite, hectonite, and anhydrous silicic acid.

Commercially available viscosity modifiers highly useful herein include Carbomers with trade names Carbopol® 934, Carbopol® 940, Carbopol® 950, Carbopol® 980, and Carbopol® 981, all available from B. F. Goodrich Company, acrylates/steareth-20 methacrylate copolymer with trade name ACRYSOL™ 22 available from Rohm and Hass, nonoxynyl hydroxyethylcellulose with trade name Amercell™ POLYMER HM-1500 available from Amerchol, methylcellulose with trade name BENECEL®, hydroxyethyl cellulose with trade name NATROSOL®, hydroxypropyl cellulose with trade name KLUCEL®, cetyl hydroxyethyl cellulose with trade name POLYSURF® 67, all supplied by Hercules, ethylene oxide and/or propylene oxide based polymers with trade names CARBOWAX® PEGs, POLYOX WASRs, and UCON® FLUIDS, all supplied by Amerchol.

Other optional suspending agents include crystalline suspending agents which can be categorized as acyl derivatives, long chain amine oxides, and mixtures thereof. These suspending agents are described in U.S. Pat. No. 4,741,855.

These suspending agents include ethylene glycol esters of fatty acids in one aspect having from about 16 to about 22 carbon atoms. In one aspect, useful suspending agents include ethylene glycol stearates, both mono and distearate, but in one aspect, the distearate containing less than about 7% of the mono stearate. Other suitable suspending agents include alkanol amides of fatty acids, having from about 16 to about 22 carbon atoms, or even about 16 to 18 carbon atoms, examples of which include stearic monoethanolamide, stearic diethanolamide, stearic monoisopropanolamide and stearic monoethanolamide stearate. Other long chain acyl derivatives include long chain esters of long chain fatty acids (e.g., stearyl stearate, cetyl palmitate, etc.); long chain esters of long chain alkanol amides (e.g., stearamide diethanolamide distearate, stearamide monoethanolamide stearate); and glyceryl esters (e.g., glyceryl distearate, trihydroxystearin, tribehenin) a commercial example of which is Thixcin® R available from Elementis. Long chain acyl derivatives, ethylene glycol esters of long chain carboxylic acids, long chain amine oxides, and alkanol amides of long chain carboxylic acids in addition to the materials listed above may be used as suspending agents.

Other long chain acyl derivatives suitable for use as suspending agents include N,N-dihydrocarbyl amido benzoic acid and soluble salts thereof (e.g., Na, K), particularly N,N-di(hydrogenated) C16, C18 and tallow amido benzoic acid species of this family, which are commercially available from Stepan Company (Northfield, Ill., USA).

Examples of suitable long chain amine oxides for use as suspending agents include alkyl dimethyl amine oxides, e.g., stearyl dimethyl amine oxide.

Other suitable suspending agents include primary amines having a fatty alkyl moiety having at least about 16 carbon atoms, examples of which include palmitamine or stearamine, and secondary amines having two fatty alkyl moieties each having at least about 12 carbon atoms, examples of which include dipalmitoylamine or di(hydrogenated tallow)amine. Still other suitable suspending agents include di(hydrogenated tallow)phthalic acid amide, and crosslinked maleic anhydride-methyl vinyl ether copolymer.

5. Deposition Aids

The hair care compositions of the present invention may further comprise a deposition aid, such as a cationic polymer. Cationic polymers useful herein are those having an average molecular weight of at least about 5,000, alternatively from about 10,000 to about 10 million, and alternatively from about 100,000 to about 2 million.

Suitable cationic polymers include, for example, copolymers of vinyl monomers having cationic amine or quaternary ammonium functionalities with water soluble spacer monomers such as acrylamide, methacrylamide, alkyl and dialkyl acrylamides, alkyl and dialkyl methacrylamides, alkyl acrylate, alkyl methacrylate, vinyl caprolactone, and vinyl pyrrolidone. Other suitable spacer monomers include vinyl esters, vinyl alcohol (made by hydrolysis of polyvinyl acetate), maleic anhydride, propylene glycol, and ethylene glycol. Other suitable cationic polymers useful herein include, for example, cationic celluloses, cationic starches, and cationic guar gums.

The cationic polymer can be included in the hair care compositions of the present invention at a level of from about 0.001 wt % to about 10 wt %. In one embodiment, the cationic polymer is present in an amount up to about 5 wt % based on the weight of the composition.

6. Benefit Agents

In an embodiment, the hair care composition further comprises one or more additional benefit agents. The benefit agents comprise a material selected from the group consisting of anti-dandruff agents, vitamins, lipid soluble vitamins, chelants, perfumes, brighteners, enzymes, sensates, attractants, anti-bacterial agents, dyes, pigments, bleaches, and mixtures thereof.

In one aspect said benefit agent may comprise an anti-dandruff agent. Such anti-dandruff particulate should be physically and chemically compatible with the components of the composition, and should not otherwise unduly impair product stability, aesthetics or performance

According to an embodiment, the hair care composition comprises an anti-dandruff active, which may be an anti-dandruff active particulate. In an embodiment, the anti-dandruff active is selected from the group consisting of: pyridinethione salts; azoles, such as ketoconazole, econazole, and elubiol; selenium sulphide; particulate sulfur; keratolytic agents such as salicylic acid; and mixtures thereof. In an embodiment, the anti-dandruff particulate is a pyridinethione salt.

Pyridinethione particulates are suitable particulate anti-dandruff actives. In an embodiment, the anti-dandruff active is a 1-hydroxy-2-pyridinethione salt and is in particulate form. In an embodiment, the concentration of pyridinethione anti-dandruff particulate ranges from about 0.01 wt % to about 5 wt %, or from about 0.1 wt % to about 3 wt %, or from about 0.1 wt % to about 2 wt %. In an embodiment, the pyridinethione salts are those formed from heavy metals such as zinc, tin, cadmium, magnesium, aluminium and zirconium, generally zinc, typically the zinc salt of 1-hydroxy-2-pyridinethione (known as “zinc pyridinethione” or “ZPT”), commonly 1-hydroxy-2-pyridinethione salts in platelet particle form. In an embodiment, the 1-hydroxy-2-pyridinethione salts in platelet particle form have an average particle size of up to about 20 microns, or up to about 5 microns, or up to about 2.5 microns. Salts formed from other cations, such as sodium, may also be suitable. Pyridinethione anti-dandruff actives are described, for example, in U.S. Pat. No. 2,809,971; U.S. Pat. No. 3,236,733; U.S. Pat. No. 3,753,196; U.S. Pat. No. 3,761,418; U.S. Pat. No. 4,345,080; U.S. Pat. No. 4,323,683; U.S. Pat. No. 4,379,753; and U.S. Pat. No. 4,470,982.

In an embodiment, in addition to the anti-dandruff active selected from polyvalent metal salts of pyrithione, the composition further comprises one or more anti-fungal and/or anti-microbial actives. In an embodiment, the anti-microbial active is selected from the group consisting of: coal tar, sulfur, fcharcoal, whitfield's ointment, castellani's paint, aluminum chloride, gentian violet, octopirox (piroctone olamine), ciclopirox olamine, undecylenic acid and its metal salts, potassium permanganate, selenium sulphide, sodium thiosulfate, propylene glycol, oil of bitter orange, urea preparations, griseofulvin, 8-hydroxyquinoline ciloquinol, thiobendazole, thiocarbamates, haloprogin, polyenes, hydroxypyridone, morpholine, benzylamine, allylamines (such as terbinafine), tea tree oil, clove leaf oil, coriander, palmarosa, berberine, thyme red, cinnamon oil, cinnamic aldehyde, citronellic acid, hinokitol, ichthyol pale, Sensiva SC-50, Elestab HP-100, azelaic acid, lyticase, iodopropynyl butylcarbamate (IPBC), isothiazalinones such as octyl isothiazalinone, and azoles, and mixtures thereof. In an embodiment, the anti-microbial is selected from the group consisting of: itraconazole, ketoconazole, selenium sulphide, coal tar, and mixtures thereof.

In an embodiment, the azole anti-microbials is an imidazole selected from the group consisting of: benzimidazole, benzothiazole, bifonazole, butaconazole nitrate, climbazole, clotrimazole, croconazole, eberconazole, econazole, elubiol, fenticonazole, fluconazole, flutimazole, isoconazole, ketoconazole, lanoconazole, metronidazole, miconazole, neticonazole, omoconazole, oxiconazole nitrate, sertaconazole, sulconazole nitrate, tioconazole, thiazole, and mixtures thereof, or the azole anti-microbials is a triazole selected from the group consisting of: terconazole, itraconazole, and mixtures thereof. When present in the hair care composition, the azole anti-microbial active is included in an amount of from about 0.01 wt % to about 5 wt %, or from about 0.1 wt % to about 3 wt %, or from about 0.3 wt % to about 2 wt %. In an embodiment, the azole anti-microbial active is ketoconazole. In an embodiment, the sole anti-microbial active is ketoconazole.

Embodiments of the hair care composition may also comprise a combination of anti-microbial actives. In an embodiment, the combination of anti-microbial active is selected from the group of combinations consisting of: octopirox and zinc pyrithione, pine tar and sulfur, salicylic acid and zinc pyrithione, salicylic acid and elubiol, zinc pyrithione and elubiol, zinc pyrithione and climbasole, octopirox and climbasole, salicylic acid and octopirox, and mixtures thereof.

In an embodiment, the composition comprises an effective amount of a zinc-containing layered material. In an embodiment, the composition comprises from about 0.001 wt % to about 10 wt %, or from about 0.01 wt % to about 7 wt %, or from about 0.1 wt % to about 5 wt % of a zinc-containing layered material, by total weight of the composition.

Zinc-containing layered materials may be those with crystal growth primarily occurring in two dimensions. It is conventional to describe layer structures as not only those in which all the atoms are incorporated in well-defined layers, but also those in which there are ions or molecules between the layers, called gallery ions (A. F. Wells “Structural Inorganic Chemistry” Clarendon Press, 1975). Zinc-containing layered materials (ZLMs) may have zinc incorporated in the layers and/or be components of the gallery ions. The following classes of ZLMs represent relatively common examples of the general category and are not intended to be limiting as to the broader scope of materials which fit this definition.

Many ZLMs occur naturally as minerals. In an embodiment, the ZLM is selected from the group consisting of: hydrozincite (zinc carbonate hydroxide), aurichalcite (zinc copper carbonate hydroxide), rosasite (copper zinc carbonate hydroxide), and mixtures thereof. Related minerals that are zinc-containing may also be included in the composition. Natural ZLMs can also occur wherein anionic layer species such as clay-type minerals (e.g., phyllosilicates) contain ion-exchanged zinc gallery ions. All of these natural materials can also be obtained synthetically or formed in situ in a composition or during a production process.

Another common class of ZLMs, which are often, but not always, synthetic, is layered double hydroxides. In an embodiment, the ZLM is a layered double hydroxide conforming to the formula [M²⁺₁₋ₓM³⁺ₓ(OH)₂]^{x+}A^{m-}_{x/m}.nH₂O wherein some or all of the divalent ions (M²⁺) are zinc ions (Crepaldi, E L, Pava, P C, Tronto, J, Valim, J B J. Colloid Interfac. Sci. 2002, 248, 429-42).

Yet another class of ZLMs can be prepared called hydroxy double salts (Morioka, H., Tagaya, H., Karasu, M, Kadokawa, J, Chiba, K Inorg. Chem. 1999, 38, 4211-6). In an embodiment, the ZLM is a hydroxy double salt conforming to the formula [M²⁺₁₋ₓM²⁺₁₊ₓ(OH)_{3(1-y)}]⁺Aⁿ⁻_{(1=3y)/n}.nH₂O where the two metal ions (M²⁺) may be the same or different. If they are the same and represented by zinc, the formula simplifies to [Zn₁₊ₓ(OH)₂]^{2x+}2x A⁻.nH₂O. This latter formula represents (where x=0.4) materials such as zinc hydroxychloride and zinc hydroxynitrate. In an embodiment, the ZLM is zinc hydroxychloride and/or zinc hydroxynitrate. These are related to hydrozincite as well wherein a divalent anion replaces the monovalent anion. These materials can also be formed in situ in a composition or in or during a production process.

In embodiments having a zinc-containing layered material and a pyrithione or polyvalent metal salt of pyrithione, the ratio of zinc-containing layered material to pyrithione or a polyvalent metal salt of pyrithione is from about 5:100 to about 10:1, or from about 2:10 to about 5:1, or from about 1:2 to about 3:1.

The on-scalp deposition of the anti-dandruff active is at least about 1 microgram/cm². The on-scalp deposition of the anti-dandruff active is important in view of ensuring that the anti-dandruff active reaches the scalp where it is able to perform its function. In an embodiment, the deposition of the anti-dandruff active on the scalp is at least about 1.5 microgram/cm², or at least about 2.5 microgram/cm², or at least about 3 microgram/cm², or at least about 4 microgram/cm², or at least about 6 microgram/cm², or at least about 7 microgram/cm², or at least about 8 microgram/cm², or at least about 8 microgram/cm², or at least about 10 microgram/cm². The on-scalp deposition of the anti-dandruff active is measured by having the hair of individuals washed with a composition comprising an anti-dandruff active, for example a composition pursuant to the present invention, by trained a cosmetician according to a conventional washing protocol. The hair is then parted on an area of the scalp to allow an open-ended glass cylinder to be held on the surface while an aliquot of an extraction solution is added and agitated prior to recovery and analytical determination of anti-dandruff active content by conventional methodology, such as HPLC.

### Test Methods

### A. Molecular Weight Distribution

Weight-average molecular weight (M_{w}) values of the glyceride copolymers are determined as follows. Sample molecular weights are determined on an Agilent 1260 HPLC system equipped with autosampler, column oven, and refractive index detector. The operating system is OpenLAB CDS ChemStation Workstation (A.01.03). Data storage and analysis are performed with Cirrus GPC offline, GPC/SEC Software for ChemStation, version 3.4. Chromatographic conditions are given in Table 3. In carrying out the calculation, the results are calibrated using polystyrene reference samples having known molecular weights. Measurements of M_{w} values vary by 5% or less. The molecular weight analyses are determined using a chloroform mobile phase.

TABLE 3

| Parameter | Conditions |
|---|---|
| Column Set | Three ResiPore columns (Agilent #1113-6300) in |
| | series with guard column (Agilent #1113-1300) |
| | Particle size: 3 μm |
| | Column dimensions: 300 × 7.5 mm |
| Mobile Phase | Chloroform |
| Flow Rate | 1 mL/min, needle wash is included |
| Column Temperature | 40° C. |
| Injection Volume | 20 μL |
| Detector | Refractive Index |
| Detector Temperature | 40° C. |

Table 4 shows the molecular weights and the retention times of the polystyrene standards.

TABLE 4

| Standard Number | Average Reported MW | Retention Time (min) |
|---|---|---|
| 1 | 150,000 | 19.11 |
| 2 | 100,000 | 19.63 |
| 3 | 70,000 | 20.43 |
| 4 | 50,000 | 20.79 |
| 5 | 30,000 | 21.76 |
| 6 | 9,000 | 23.27 |
| 7 | 5,000 | 23.86 |
| 8 | 1,000 | 27.20 |
| 9 | 500 | 28.48 |

### B. Iodine Value

Another aspect of the invention provides a method to measure the iodine value of the glyceride copolymer. The iodine value is determined using AOCS Official Method Cd 1-25 with the following modifications: carbon tetrachloride solvent is replaced with chloroform (25 ml), an accuracy check sample (oleic acid 99%, Sigma-Aldrich; IV=89.86±2.00 cg/g) is added to the sample set, and the reported IV is corrected for minor contribution from olefins identified when determining the free hydrocarbon content of the glyceride copolymer.

### C. Gas Chromatographic Analysis of Fatty Acid Residues in Glyceride Copolymer

The final glyceride oligomer products described in Synthetic Examples 4, 5, and 6 are analyzed by gas chromatography after olefins are vacuum distilled to below 1% by weight and the resulting oligomer products are trans-esterified to methyl esters by the following procedure.

A sample 0.10±0.01 g is weighed into a 20 mL scintillation vial. A 1% solution of sodium methoxide in methanol (1.0 mL) is transferred by pipette into the vial and the vial is capped. The capped vial is placed in a sample shaker and shaken at 250 rpm and 60° C. until the sample is completely homogeneous and clear. The sample is removed from the shaker and 5 ml of brine solution followed by 5 ml of ethyl acetate are added by pipette. The vial is vortex mixed for one minute to thoroughly to mix the solution thoroughly. The mixed solution is allowed to sit until the two layers separated. The top (ethyl acetate) layer (1 mL) is transferred to a vial for gas chromatographic analysis. Their normalized compositions, based on a select group of components, are shown in Table 9 in units of wt %.

Gas chromatographic data are collected using an Agilent 6850 instrument equipped with an Agilent DB-WAXETR column (122-7332E, 30 m×250 um×0.25 um film thickness) and a Flame Ionization Detector. The methods and the conditions used are described as follows: The GC method “Fast_FAME.M” is used for the analyses of all samples in Synthetic Examples 1 through 7.

Method FAST_FAME.M
OVEN

| Initial temp: | | 40° C. (On) | |
|---|---|---|---|
| Initial time: | | 0.00 min | |
| Ramps: | | | |
| | Rate | Final temp | Final time |
| # | (° C./min) | (° C.) | (min) |
| 1 | 20.00 | 240 | 20.00 |
| 2 | 0 (Off) | | |

| | |
|---|---|
| Post temp: | 0° C. |
| Post time: | 0.00 min |
| Run time: | 30.00 min |
| Maximum temp: | 260° C. |
| Equilibration time: | 0.10 min |

INLET (SPLIT/SPLITLESS)

| | |
|---|---|
| Mode: | Split |
| Initial temp: | 250° C. (On) |
| Pressure: | 6.06 psi (On) |
| Split ratio: | 150:1 |
| Split flow: | 149.9 mL/min |
| Total flow: | 157.5 mL/min |
| Gas saver: | On |
| Saver flow: | 20.0 mL/min |
| Saver time: | 2.00 min |
| Gas type: | Hydrogen |

DETECTOR (FID)

| | |
|---|---|
| Temperature: | 300° C. (On) |
| Hydrogen flow: | 40.0 mL/min (On) |
| Air flow: | 450.0 mL/min (On) |
| Mode: | Constant makeup flow |
| Makeup flow: | 30.0 mL/min (On) |
| Makeup Gas Type: | Nitrogen |
| Flame: | On |
| Electrometer: | On |
| Lit offset: | 2.0 pA |

COLUMN

| | |
|---|---|
| Capillary Column | |
| Model Number: | DB-WAXETR |
| Description: | 122-7332E |
| Max temperature: | 260° C. |
| Nominal length: | 30.0 m |
| Nominal diameter: | 250.00 um |
| Nominal film thickness: | 0.25 um |
| Mode: | constant flow |
| Initial flow: | 1.0 mL/min |
| Nominal init pressure: | 6.06 psi |
| Average velocity: | 29 cm/sec |
| Source: | Inlet |
| Outlet: | Detector |
| Outlet pressure: | ambient |

SIGNAL

| | |
|---|---|
| Data rate: | 20 Hz |
| Type: | detector |
| Save Data: | On |

INJECTOR

| Sample pre-washes: | 3 |
|---|---|
| Sample pumps: | 1 |
| Sample volume (uL): | 1.000 |
| Syringe size (uL): | 10.0 |
| Pre washes from bottle A: | 3 |
| Pre washes from bottle B: | 3 |
| Post washes from bottle A: | 3 |
| Post washes from bottle B: | 3 |
| Viscosity delay (seconds): | 0 |
| Pre injection dwell (min): | 0.00 |
| Post injection dwell (min): | 0.00 |
| Sample skim depth (mm): | 0.0 (Off) |
| NanoLiter Adapter Installed | |
| Solvent Wash Mode: | A, B |
| Plunger Speed: | Fast |
| Solvent saver: | Off |

### D. Free Hydrocarbon Content

Another aspect of this invention provides a method to determine the free hydrocarbon content of the glyceride copolymer. The method combines gas chromatography/mass spectroscopy (GC/MS) to confirm identity of the free hydrocarbon homologs and gas chromatography with flame ionization detection (GC/FID) to quantify the free hydrocarbon present.

Sample Prep: The sample to be analyzed is typically trans-esterified by diluting (e.g. 400:1) in methanolic KOH (e.g. 0.1N) and heating in a closed container until the reaction is complete (i.e. 90° C. for 30 min.) then cooled to room temperature. The sample solution could then be treated with 15% boron tri-fluoride in methanol and again heated in a closed vessel until the reaction is complete (i.e. at 60° C. for 30 min) both to acidify (methyl orange-red) and to methylate any free acid present in the sample. After allowing to cool to room temperature, the reaction is quenched by addition of saturated NaCl in water. An organic extraction solvent such as cyclohexane containing a known level internal standard (e.g. 150 ppm dimethyl adipate) is then added to the vial and mixed well. After the layers separated, a portion of the organic phase is transferred to a vial suitable for injection to the gas chromatograph. This sample extraction solution is analyzed by GC/MS to confirm identification of peaks matching hydrocarbon retention times by comparing to reference spectra and then by GC/FID to calculate concentration of hydrocarbons by comparison to standard FID response factors.

A hydrocarbon standard of known concentrations, such as 50 ppm each, of typically observed hydrocarbon compounds (i.e. 1-dodecene, 1-tridecene, 1-tetradecene, 1-pentadecene, 1-hexadecene, 1-heptadecene, 1-octadecene, dodecane, tridecane, tetradecane, pentadecane, hexadecane, heptadecane and octadecane) is prepared by dilution in the same solvent containing internal standard as is used to extract the sample reaction mixture. This hydrocarbon standard is analyzed by GC/MS to generate retention times and reference spectra and then by GC/FID to generate retention times and response factors.

GC/MS: An Agilent 7890 GC equipped with a split/splitless injection port coupled with a Waters QuattroMicroGC mass spectrometer set up in EI+ ionization mode is used to carry out qualitative identification of peaks observed. A non-polar DB1-HT column (15 m×0.25 mm×0.1 um df) is installed with 1.4 mL/min helium carrier gas. In separate runs, 1 uL of the hydrocarbon standard and sample extract solution are injected to a 300° injection port with a split ratio of 25:1. The oven is held at 40° C. for 1 minute then ramped 15 C°/minute to a final temperature of 325° C. which is held for 10 minutes resulting in a total run time of 30 minutes. The transfer line is kept at 330° C. and the temperature of the EI source is 230° C. The ionization energy is set at 70 eV and the scan range is 35-550 m/z.

GC/FID: An Agilent 7890 GC equipped with a split/splitless injection port and a flame ionization detector is used for quantitative analyses. A non-polar DB1-HT column (5 m×0.25 mm×0.1 um df) is installed with 1.4 mL/min helium carrier gas. In separate runs, 1 uL of the hydrocarbon standard and sample extract solution is injected to a 330° injection port with a split ratio of 100:1. The oven is held at 40° C. for 0.5 minutes then ramped at 40 C°/minute to a final temperature of 380° C. which is held for 3 minutes resulting in a total run time of 12 minutes. The FID is kept at 380° C. with 40 mL/minute hydrogen gas flow and 450 mL/min air flow. Make up gas is helium at 25 mL/min. The hydrocarbon standard is used to create a calibration table in the Chemstation Data Analysis software including known concentrations to generate response factors. These response factors are applied to the corresponding peaks in the sample chromatogram to calculate total amount of free hydrocarbon found in each sample.

### E. Wet and Dry Combing Test Method

This test method is designed to allow for a subjective evaluation of the basic performance of rinse-off conditioners for both wet combing and dry combing efficacy. In a typical test, 3 to 5 separate formulations may be assessed for their performance. The assessment may include control treatments containing no silicone and an elevated silicone level to facilitate differentiation of performance. The substrate is virgin brown hair obtainable from a variety of sources that is screened to insure uniformity and lack of meaningful surface damage or low lift bleach damaged hair.

a. Treatment Procedure

Four to five 4 gram, 8 inch length switches are combined in a hair switch holder, wet for ten seconds with manipulation with 39±1° C. water of medium hardness (3-10 gpg) to ensure complete and even wetting. The switch is deliquored lightly and clarifying shampoo (i.e., containing no conditioning materials) is applied uniformly over the length of the combined switches from one inch below the holder towards the tip at a level of 0.1 gram product per one gram of dry hair (0.1 g/g of hair or 2 g for 20 g hair). The switch combo is lathered for 30 seconds by a rubbing motion typical of that used by consumers and rinsed with 39±1° C. water flowing at 1.5 gal/min (with the hair being manipulated) for a further 30 seconds to ensure completeness. This step is repeated. The conditioner treatments are applied in the same way as clarifying shampoo above (0.1 g/g of hair or reduced to 0.05 g/g of hair for more concentrated prototypes), milked throughout the switch combo for 30 seconds, left to sit for a further 30 seconds, and rinsed thoroughly with manipulation, again for 30 seconds. The switches are deliquored lightly, separated from each other, hung on a rack so that they are not in contact, and detangled with a wide tooth comb.

b. Grading Procedures

For wet combing evaluations using trained graders, the switches are separated on the rack into the five sets with one switch from each treatment included in the grading set. Only two combing evaluations are performed on each switch. The graders are asked to compare the treatments by combing with a narrow tooth nylon comb typical of those used by consumers and rate the ease/difficulty on a zero to ten scale. Ten separate evaluations are collected and the results analyzed by a statistical analysis package for establishing statistical significance. Statistical significance in differences between treatments is determined using Statgraphics Plus 5.1.

For dry combing evaluations, the switches from above are moved into a controlled temperature and humidity room (22° C./50% RH) and allowed to dry overnight. They remain separated as above and panelists are requested to evaluate dry conditioning performance by making three assessments; dry combing ease of the middle of the switch, dry combing ease of the tips, and a tactile assessment of tip feel. The same ten point scale is used for these comparisons. Again, only two panelists make an assessment of each switch set. Statistical analysis to separate differences is performed using the same method as above.

### F. Friction Reduction on Dry Hair (IFM)

Dry conditioning performance is also evaluated via hair friction force measurements with an Instron Tester instrument (Instron 5542, Instron, Inc.; Canton, Mass., USA). In a typical procedure, hair switches are first prepared according to treatment protocol C and dried overnight in a controlled temperature and humidity room (22° C./50% RH). The friction force (grams) between the hair surface and an urethane pad along the hair is measured, with three measurements per switch.

### G. Wet and Dry Friction Conditioning Test

This wet friction test determines the amount of conditioning provided by hair care composition products as measured by the force required to pull hair through an Instron equipped with two combs while wet. The operator ranks and balances the 4 g, 8 in. bleached hair switches for base line condition by using the Instron machine to determine a baseline force. The operator then applies a measured amount of conditioner to a hair switch that is pre-washed with a clarifying shampoo, distributes the product evenly through the switch. For conditioner testing, it is preferred to prewash the hair switch with a shampoo, rinse and then apply the conditioner. The wet forces are then measured after the product is rinsed using the Instron machine equipped with two combs. After wet combing testing, the hair switches are allowed to dry and equilibrate in a controlled temperature and humidity room (22° C./50% RH) overnight. The dry forces are then measured using same Instron equipped with two combs in a controlled temperature and humidity room (22° C./50% RH). Each test product is applied to a total of 3 switches. The data is then analyzed using standard statistical methods.

### H. Dry Conditioning Tests

This inter-fiber friction test determines the amount of friction on the hair provided by a conditioner as measured by the force required to move hair up and down past each other. This method emulates the motion of rubbing hair between the thumb and index finger in an up and down direction of the treated hair switch. The operator ranks and balances the 4 g, 8 in. hair switches for base line condition by using an Instron machine. The operator then applies a measured amount of hair care composition to a hair switch, distributes the product evenly through the switch and rinses as per the protocol. For conditioner testing, it is preferred to prewash the hair switch with a shampoo, rinse and then apply the conditioner. Wet switches are then allowed to dry overnight and evaluated the next day for friction force using the Instron machine. Each test product is applied to a total of 4 switches. The data is then analyzed using standard statistical methods.

### EXAMPLES

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

Non-limiting examples of product formulations disclosed in the present specification are summarized below.

### Synthetic Example 1—Reaction with Butenylyzed Canola Oil (BCO): Effect of BCO Content

The experimental apparatus consists of a three-necked round-bottom flask equipped with a magnetic stir bar, a septum cap, and an outlet to a vacuum system. External heating is provided via a silicone oil bath. The septum is used to add metathesis catalyst and withdraw samples. The vacuum system consisted of a TEFLON diaphragm pump and a pressure controller.

Butenylyzed canola oil (BCO) is made by cross-metathesizing canola oil (Wesson) with 1-butene (1 mol of 1-butene per mol of C═C double bonds in the oil) according to the methods described in U.S. Pat. No. 8,957,268. The BCO is mixed with canola oil (Wesson) and charged to a 500-mL round-bottom flask. The oil mixture is purged with nitrogen gas (Airgas, UHP) for about 15 minutes. The reaction flask is heated to about 70° C. and evacuated to the desired pressure (see below: 200 or 450 torr absolute.) A toluene (Sigma-Aldrich, anhydrous 99.8%) solution of C827 metathesis catalyst (10 mg/mL; Materia, Inc., Pasadena, Calif., USA) is added to the oil mixture to achieve a catalyst level of 100 ppmwt. The reaction is held at 70° C. while maintaining a dynamic vacuum at the desired pressure for 2 hours. A small sample of the reaction mixture is removed by syringe, quenched with ethyl vinyl ether (Sigma-Aldrich), and analyzed by GPC to determine the weight-average molecular weight (M_{w}) of the resulting glyceride oligomers.

Table 5 shows the resulting M_{w} for different reactions, where the percentage of BCO is increased. The percentage of BCO reported is a weight percentage of BCO relative to the total weight of oil (BCO and canola oil combined). The molecular weights are reported in units of g/mol.

TABLE 5

| | M_{w} | M_{w} |
|---|---|---|
| Percentage BCO | 450 Torr (absolute) | 200 Torr (absolute) |
| (wt %) | Experiments | Experiments |
| 0 | 11,700 | 12,300 |
| 10 | 12,800 | 13,100 |
| 30 | 13,600 | 14,800 |
| 50 | 14,400 | 18,000 |
| 70 | 14,100 | 22,500 |
| 90 | 14,500 | — |
| 100 | 25,900 | 56,600 |

### Synthetic Example 2—Reaction with Butenylyzed Canola Oil (BCO): Effect of Reaction Time

Using the same apparatus and procedures as those described in Synthetic Example 1, 50 wt %/50 wt % mixtures of BCO and canola oil are reacted for four hours while maintaining a dynamic vacuum at either 200 or 450 torr (absolute) with samples being taken hourly. Table 6 shows the molecular weight (M_{w}) over time. The molecular weight (M_{w}) is reported in units of g/mol.

TABLE 6

| Time | M_{w} | M_{w} |
|---|---|---|
| (hr) | 450 Torr (absolute) Experiments | 200 Torr (absolute) Experiments |
| 1 | 13,600 | 16,100 |
| 2 | 13,600 | 18,000 |
| 3 | 13,100 | 19,000 |
| 4 | 13,000 | 20,000 |

### Synthetic Example 3—Cross-Metathesis of Canola Oil with Butenylyzed Palm Oil (BPO): Effect of Feedstock Composition

Using the same apparatus and procedures as those described in Synthetic Example 1, three different mixtures of BPO (Wilmar) and canola oil are reacted for two hours. Table 7 shows the molecular weight (Mw) after two hours. The molecular weight (M_{w}) is reported in units of g/mol.

TABLE 7

| | Percentage BPO | M_{w} 200 Torr |
|---|---|---|
| Example | (wt %) | (absolute) Experiment |
| Synthetic Example 3A | 15 | 9,400 |
| Synthetic Example 3B | 25 | 8,100 |
| Synthetic Example 3C | 35 | 5,900 |

### Synthetic Example 4—Cross-Metathesis of Canola with Butenylyzed Canola Oil (BCO) on One-Kilogram Scale with Catalyst Removal and Olefin Stripping

Using a similar metathesis procedure and apparatus to the one described in Synthetic Example 1, a 1 kg mixture of BCO and canola oil (50 wt %/50 wt %) is reacted for two hours. Catalyst removal is accomplished by THMP treatment. THMP treatments consists of adding 1 M tris(hydroxymethyl)phosphine (THMP, 1.0 M, 50 mol THMP/mol C827) in water, stirring at ambient temperature for 2 hours, and then washing the product with water (2×100 mL) in a separatory funnel. Olefins are removed by vacuum distillation in a 1 L three-neck round-bottom equipped with a short-path distillation head; a condenser chilled to 5° C.; a 20 mL round bottom flask chiller with dry-ice/isopropanol; a magnetic stir bar; and thermometers to measure liquid temperature and vapor temperature. Heating is supplied through a resistive heating mantle. Vacuum is supplied by a two-stage rotary vane vacuum pump. The bulk of olefinic material is removed by gradually increasing the heat input. The final pressure is about 0.2 torr absolute and the final liquid temperature is 195° C. The olefin content is less than 1% by mass and the M_{w} of the glyceride oligomer is 16,700 g/mol. A sample of the final product is trans-esterified and analyzed by GC to determine the Fatty Acid Residues as described above. See Table 8 below.

### Synthetic Example 5—Cross-Metathesis of Soybean Oil with Butenylyzed Soybean Oil (BSO) on a Two-Kilogram Scale with Catalyst Removal and Olefin Stripping

Using the same procedure and an apparatus similar to that described in Synthetic Example 1 except that a 3 L flask is used in place of the 500 mL flask, a 1 kg, 50/50 wt % mixture of butenylyzed soybean oil and soybean oil (Costco) is reacted for about four hours using 100 ppm wt C827 catalyst. An additional 40 ppm of catalyst is added and after about two more hours the reaction is quenched with ethyl vinyl ether. Olefin by-products and traces of residual water are removed from a 265 g sample of the product by a similar distillation procedure and apparatus as described in Synthetic Example 4. The final pressure is about 0.1 torr absolute and the final liquid temperature is 195° C. The olefin content is less than 1% by mass. A sample of the final product is trans-esterified and analyzed by GC to determine the Fatty Acid Residues as described above. See Table 8 below.

### Synthetic Example 6—Cross-Metathesis of Canola Oil with Butenylyzed Canola Oil (BCO) on a Twelve-Kilogram Scale with Catalyst Removal and Olefin Stripping

This example is conducted in a 5 gallon Stainless Steel Reactor (Parr) equipped with an impeller, a port for air-free catalyst addition, and a Strahman valve for sampling. The reactor system is completely purged with nitrogen before beginning.

The BCO (6.16 kg) is produced by a procedure similar to that used in Synthetic Example 1 and mixed with canola oil (6.12 kg) and charged to the reactor. The oil mixture is stirred at 200 rpm while purging with nitrogen gas for about 30 minutes through a dip tube at a rate of 0.5 SCFM. The reactor is evacuated to 200 torr (absolute) and heated to 70° C. The C827 metathesis catalyst (1.0 g, Materia, Inc., Pasadena, Calif., USA) is suspended in canola oil (50 mL) and added to the oil mixture. The reaction is maintained at 70° C. and at 200 torr for four hours. An additional charge of C827 catalyst (0.25 g) suspended in canola oil (50 mL) is added to the reaction. After an additional two hours, the reactor is back filled with nitrogen.

Catalyst removal is conducted in a 5 gallon jacketed glass reactor equipped with an agitator, a bottom drain valve, and ports for adding reagents. A 0.12 M aqueous solution of THMP (0.31 kg) is charged to the glass reactor and pre-heated to about 90° C. The crude metathesis reaction product, still at 70° C., is transferred to the glass reactor and the mixture is stirred (150 rpm) at about 80-90° C. for 20 minutes. The following wash procedure is done twice. Deionized water (1.9 kg at 60° C.) is added to the reactor which is heated to 80-90° C. and the resulting mixture is stirred (100 rpm) for 20 minutes. The stirrer is stopped and the reactor contents are allowed to settle for 16 hours at a constant temperature of 80-90° C. The bottom aqueous layer is carefully drained off. Following the second wash, the washed product is cooled and then drained to a container.

The washed product is divided into two portions to remove olefins and residual water, which is done using a similar distillation procedure and apparatus as described in Synthetic Example 4. The final distillation pressure is about 0.1 torr absolute and the final liquid temperature is about 190° C. Following distillation, the two portions are recombined. A small sample of the recombined product is trans-esterified and analyzed by GC to determine the Fatty Acid Residues as described above. See Table 8 below.

The fatty acid residues in the final glyceride oligomer products produced in Synthetic Examples 4, 5, and 6 are analyzed by the method described above after olefins are vacuum distilled to below 1% by weight. The C₁₀₋₁₄ unsaturated fatty acid esters, C₁₀₋₁₃ unsaturated fatty acid esters, and C₁₀₋₁₁ unsaturated fatty acid esters are calculated and are reported in Table 9 below.

TABLE 8

| | Synthetic | Synthetic | |
|---|---|---|---|
| Fatty Acid | Example 4 | Example 5 | Synthetic |
| Methyl Ester | Product | Product | Example 6 |
| Component | (wt %) | (wt %) | Product (wt %) |
| C10:1 | 6.72 | 2.97 | 4.58 |
| C12:1 | 7.33 | 4.77 | 6.25 |
| C13:2 | 1.33 | 0.71 | 0.72 |
| C15:1 | 5.05 | 12.21 | 5.05 |
| C16:0 | 6.12 | 14.69 | 5.65 |
| C16:1 | 1.08 | 0.43 | 1.06 |
| C18:0 | 2.65 | 6.05 | 2.58 |
| C18:1 | 19.52 | 6.31 | 19.80 |
| C18:2 | 1.33 | 3.46 | 0.89 |
| C18:3 | 0.39 | 0.42 | 0.27 |
| C20:0 | 0.85 | 0.48 | 0.90 |
| C20:1 | 1.08 | 0.29 | 1.15 |
| C21:2 | 3.59 | 1.76 | 3.61 |
| C22:0 | 0.56 | 0.08 | 0.60 |
| C18:1 diester | 29.10 | 21.84 | 29.85 |
| C20:1 diester | 3.11 | 1.02 | 3.08 |
| C21:2 diester | 5.10 | 6.40 | 4.95 |

TABLE 9

| Unsaturated | Synthetic | Synthetic | Synthetic |
|---|---|---|---|
| Fatty Acid Ester | Example 4 | Example 5 | Example 6 |
| Component | Product (wt %) | Product (wt %) | Product (wt %) |
| C₁₀₋₁₄ unsaturated | 15.38 | 8.45 | 11.55 |
| fatty acid esters | | | |
| C₁₀₋₁₃ unsaturated | 15.38 | 8.45 | 11.55 |
| fatty acid esters | | | |
| C₁₀₋₁₁ unsaturated | 6.72 | 2.97 | 4.58 |
| fatty acid esters | | | |

### Synthetic Example 7—Diene-Selective Hydrogenation of Crude Glyceride Polymer

In a 600 mL Parr reactor, 170 g of crude metathesis product from Synthetic Example 6, 170 g of n-decane (Sigma-Aldrich, anhydrous, ≧99%), and 0.60 g PRICAT 9908 (Johnson Matthey Catalysts); saturated triglyceride wax removed before reaction via a toluene wash) are purged with N₂, then H₂, for 15 minutes each, then reacted at 160° C. under 100 psig H₂ (Airgas, UHP) with 1000 rpm stirring with a gas dispersion impeller. The H₂ pressure is monitored and the reactor is refilled to 100 psig when it decreased to about 70 psig. After six hours, the reaction is cooled below 50° C. and the hydrogen is displaced by nitrogen gas. The reaction mixture is vacuum filtered through diatomaceous earth to remove the catalyst solids. Olefin by-products and n-decane are removed from the product by a similar distillation procedure and apparatus as described in Synthetic Example 4. The final distillation pressure is about 0.1 torr absolute and the final liquid temperature is 195° C. The olefin content is less than 1% by mass. A sample of the final product is trans-esterified with methanol and analyzed by GC. The level of polyunsaturated C18 fatty acid methyl esters (C18:2 plus C18:3) are reduced from 3.88% in the starting material to 1.13% and the C21:2 diester is reduced from 6.40% in the starting material to 3.72%.

### Rinse-Off Conditioner Composition Examples 1-30

In Composition Examples 15-22, the metathesized oils are emulsified with Glyceryl monooleate and Polysorbate 20 to a median particle size of about 1.2 microns prior to incorporation into the conditioner composition.

| Components | Ex. 1 | Ex. 2 | Ex. 3 | Ex. 4 | Ex. 5 | Ex. 6 | Ex. 7 |
|---|---|---|---|---|---|---|---|
| Water | q.s. | q.s. | q.s. | q.s. | q.s. | q.s. | q.s. |
| BTMS¹ | 2.3 | 2.3 | 2.3 | 2.3 | 2.3 | 2.3 | 2.3 |
| Cetyl alcohol³ | 1.1 | 1.1 | 1.1 | 1.1 | 1.1 | 1.1 | 1.1 |
| Stearyl alcohol⁴ | 2.8 | 2.8 | 2.8 | 2.8 | 2.8 | 2.8 | 2.8 |
| Aminosilicone⁵ | — | — | | | 0.5 | — | — |
| Synthetic | — | 1.0 | — | — | | — | — |
| Example 3C⁶ | | | | | | | |
| Synthetic | — | — | | 1.0 | 1.0 | 2.0 | — |
| Example 4⁷ | | | | | | | |
| Synthetic | — | — | 1.0— | — | — | — | — |
| Example 5⁸ | | | | | | | |
| Synthetic | 1.0 | — | | — | — | | — |
| Example 6⁹ | | | | | | | |
| Synthestic | — | — | — | — | — | — | 1.0 |
| Example 7¹⁰ | | | | | | | |
| Perfume | 0.5 | 0.5 | 0.5 | 0.5 | 0.5 | 0.5 | 0.5 |
| Preservatives, | Up to | Up to | Up to | Up to | Up to | Up to | Up to |
| pH, viscosity | 5% | 5% | 5% | 5% | 5% | 5% | 5% |
| adjustment | | | | | | | |

| Components | Ex. 8 | Ex. 9 | Ex. 10 | Ex. 11 | Ex. 12 | Ex. 13 | Ex. 14 |
|---|---|---|---|---|---|---|---|
| Water | q.s. | q.s. | q.s. | q.s. | q.s. | q.s. | q.s. |
| BTMAC² | 2.8 | 2.8 | 2.8 | 2.8 | 2.8 | 2.8 | 2.8 |
| Cetyl alcohol³ | 1.8 | 1.8 | 1.8 | 1.8 | 1.8 | 1.8 | 1.8 |
| Stearyl alcohol⁴ | 4.6 | 4.6 | 4.6 | 4.6 | 4.6 | 4.6 | 4.6 |
| Synthetic | 1.0 | — | — | — | — | — | — |
| Example 3C⁶ | | | | | | | |
| Synthetic | — | 1.0 | — | — | 2.0 | — | — |
| Example 4⁷ | | | | | | | |
| Synthetic | — | — | 1.0 | — | — | 2.0 | — |
| Example 5⁸ | | | | | | | |
| Synthetic | — | — | — | 1.0 | | — | — |
| Example 6⁹ | | | | | | | |
| Synthetic | — | — | — | — | — | — | 1.0 |
| Example 7¹⁰ | | | | | | | |
| Aminosilicone⁵ | — | — | 0.75 | 0.75 | — | — | — |
| Perfume | 0.5 | 0.5 | 0.5 | 0.5 | 0.5 | 0.5 | 0.5 |
| Preservatives, | Up to | Up to | Up to | Up to | Up to | Up to | Up to |
| pH, viscosity | 5% | 5% | 5% | 5% | 5% | 5% | 5% |
| adjustment | | | | | | | |

| Components | Ex. 15 | Ex. 16 | Ex. 17 | Ex. 18 | Ex. 19 | Ex. 20 | Ex. 21 | Ex. 22 |
|---|---|---|---|---|---|---|---|---|
| Water | q.s. | q.s. | q.s. | q.s. | q.s. | q.s. | q.s. | q.s. |
| SAPDMA¹¹ | 1.95 | 1.95 | 1.95 | 1.95 | 3.24 | 3.24 | 3.24 | 3.24 |
| Cetyl alcohol³ | 1.68 | 1.68 | 1.68 | 1.68 | 4.25 | 4.25 | 4.25 | 4.25 |
| Stearyl alcohol⁴ | 2.90 | 2.90 | 2.90 | 2.90 | 2.93 | 2.93 | 2.93 | 2.93 |
| Synthetic | 1.0 | — | — | — | — | — | — | — |
| Example 3C⁶ | | | | | | | | |
| Synthetic | — | 1.0 | — | — | — | 1.0 | — | 2.0 |
| Example 4⁷ | | | | | | | | |
| Synthetic | — | — | 1.0 | — | 1.0 | — | — | — |
| Example 5⁸ | | | | | | | | |
| Synthetic | — | — | — | 1.0 | — | — | — | — |
| Example 6⁹ | | | | | | | | |
| Synthetic | — | — | — | — | — | — | 2.0 | — |
| Example 7¹⁰ | | | | | | | | |
| Perfume | 0.5 | 0.5 | 0.5 | 0.5 | 0.5 | 0.5 | 0.5 | 0.5 |
| Glyceryl | 0.0085 | 0.0085 | 0.017 | 0.017 | 0.017 | 0.017 | 0.034 | 0.034 |
| Monooleate¹² | | | | | | | | |
| Polysorbate | 0.0165 | 0.0165 | 0.033 | 0.033 | 0.033 | 0.033 | 0.066 | 0.066 |
| 20¹³ | | | | | | | | |
| Preservatives, | Up to | Up to | Up to | Up to | Up to | Up to | Up to | Up to |
| pH, viscosity | 5% | 5% | 5% | 5% | 5% | 5% | 5% | 5% |
| adjustment | | | | | | | | |

| Components | Ex. 23 | Ex. 24 | Ex. 25 | Ex. 26 | Ex. 27 | Ex. 28 | Ex. 29 | Ex. 30 |
|---|---|---|---|---|---|---|---|---|
| Water | q.s. | q.s. | q.s. | q.s. | q.s. | q.s. | q.s. | q.s. |
| SAPDMA¹² | 1.95 | 1.95 | 1.95 | 1.95 | 3.24 | 3.24 | 3.24 | 3.24 |
| Cetyl alcohol³ | 1.68 | 1.68 | 1.68 | 1.68 | 4.25 | 4.25 | 4.25 | 4.25 |
| Stearyl alcohol⁴ | 2.90 | 2.90 | 2.90 | 2.90 | 2.93 | 2.93 | 2.93 | 2.93 |
| Synthetic | 1.0 | — | — | — | — | — | — | — |
| Example 3C⁶ | | | | | | | | |
| Synthetic | — | 1.0 | — | — | — | 1.0 | — | 2.0 |
| Example 4⁷ | | | | | | | | |
| Synthetic | — | — | 1.0 | — | 1.0 | — | — | — |
| Example 5⁸ | | | | | | | | |
| Synthetic | — | — | — | 1.0 | — | — | — | — |
| Example 6⁹ | | | | | | | | |
| Synthetic | — | — | — | — | — | — | 2.0 | — |
| Example 7¹⁰ | | | | | | | | |
| Perfume | 0.5 | 0.5 | 0.5 | 0.5 | 0.5 | 0.5 | 0.5 | 0.5 |
| Preservatives, | Up to | Up to | Up to | Up to | Up to | Up to | Up to | Up to |
| pH, viscosity | 5% | 5% | 5% | 5% | 5% | 5% | 5% | 5% |
| adjustment | | | | | | | | |

### Rinse of Conditioner Examples 31 and 32

| | Ex. 31 | Ex. 32 |
|---|---|---|
| Components | (Inventive) | (Comparative) |
| Water | q.s. | q.s. |
| Stearamidopropyldimethylamine¹¹ | 3.2 | 3.2 |
| Cetyl alcohol³ | 4.3 | 4.3 |
| Stearyl alcohol⁴ | 2.9 | 2.9 |
| Synthetic Example 6⁹ | 3.0 | — |
| Soybean Oil, | — | 3.0 |
| metathetical products, hydrogenated | | |
| (Hydrogenated soy polyglycerides)¹⁴ | | |
| Perfume | 0.6 | 0.6 |
| Preservatives, pH, viscosity adjustment | Up to 5% | Up to 5% |
| Glycerin¹⁵ | 3.0 | 3.0 |

Performance Using Wet and Dry Conditioning Tests

| | | |
|---|---|---|
| Wet combing mid friction (body), gf | 69.9 | 77.2 |
| Wet combing mid friction standard error | 1.7 | 2.5 |
| Wet combing peak friction (tips), gf | 22.5 | 27.3 |
| Wet combing peak friction standard error | 1.1 | 2.6 |
| Dry combing mid friction (body), gf | 8.0 | 8.3 |
| Dry combing mid friction standard error | 0.2 | 0.2 |
| Dry combing peak friction (tips), gf | 40.2 | 58.6 |
| Dry combing peak friction standard error | 1.3 | 4.2 |

For composition Examples 31-32, the Gel Matrix is prepared according to US Patent Publication No. 2014/0377205, which is incorporated herein by reference. The Gel Matrix contains all ingredients except metathesized oils (Synthetic example 6 and metathesized soybean oil), perfume and a portion of water, which constitutes 85% of total composition. 11.4% Water as in total composition is added to the Gel Matrix, followed by mixing with an overhead 4-blade mixer at 250 rpm for 20 minutes. The resulting Gel Matrix mixture is then allowed to sit at room temperature overnight before metathesized oils are incorporated. In Example 31, Synthetic Example 6 is added to the Gel Matrix mixture and mixed at 2500 rpm for 5 minutes in a SpeedMixer™ (DAC600 1 FVZ from Flack Tek Inc.). Perfume is then added and mixed at 1000 rpm for 30 seconds. In Example 32, the Soy metathesized product (CS110) is heated to 70° C. The molten Soy metathesized product is quickly added to Gel Matrix mixture preheated to 50° C. in an oven. The mixture is then mixed at 2500 rpm for 5 minutes in the SpeedMixer™. After the mixture cools down below 40° C., perfume is added and mixed at 1000 rpm for 30 seconds in the SpeedMixer™.

A small sample from both Ex.31 and Ex.32 is taken prior to perfume addition for particle size analysis. The particle size of metathesized oils in Gel Matrix can be controlled by mixing speed and time and/or by pre-emulsification, resulting in particle sizes from about 0.01 μm to about 200 μm. The particle size of metathesized oils in Gel Matrix is measured using an optical microscope optionally equipped with Differential Interference Contrast (DIC). Three representative fields of view are chosen. Eight representative particles are chosen from each field of view and the diameters of the particles are measured using a standard microscope imaging software. Mean particle size is calculated using standard statistical methods. The mean particle sizes of Ex.31 and Ex.32 are in the range of 30-40 μm.

The test results included for Examples 31 (inventive) and 32 (comparative) reflect the beneficial wet conditioning properties, as measured using the Wet Friction Conditioning Test, provided by the compositions of the present invention. The test results also reflect the dry conditioning (combing) benefits of the present compositions, as measured by the Friction Reduction on Dry Hair method, as evidenced by decreased peak friction (tips) in Example 31 versus Example 32. (It is noted that mid friction (body) statistical differences between Examples 31 and 32 are not observed due to the inherently low mid friction properties of the unconditioned hair.)

### Footnotes for Composition Examples 1-32:

¹ Behenyltrimethylammonium methylsulfate, from Feixiang
² Behenyltrimethylammonium chloride, Genamin KDMP, from Clariant
³ Cetyl alcohol, from PROCTER & GAMBLE
⁴ Stearyl alcohol, from PROCTER & GAMBLE
⁵ Y-14945; 10,000 cps aminodimethicone, from Momentive
⁶ Synthetic Example 3C in Table 7
⁷ Synthetic Example 4 in Table 8 and 9
⁸ Synthetic Example 5 in Table 8 and 9
⁹ Synthetic Example 6 in Table 8 and 9
¹⁰ Synthetic Example 7
¹¹ Stearamidopropyldimethylamine (LEXAMINE S-13), from BASF
¹² MONOMULS 90-O 18, from BASF
¹³ Polysorbate 20 from Croda
¹⁴ Elevance CS110 from Elevance Renewable Sciences
¹⁵ Glycerin USP from Kaneda Co. Ltd (Tyoko, Japan)

The hair care composition may be presented in typical hair care formulations. They may be in the form of solutions, dispersion, emulsions, powders, talcs, encapsulated spheres, spongers, solid dosage forms, foams, and other delivery mechanisms. The compositions of the embodiments of the present invention may be hair tonics, leave-on hair products such as treatment and styling products, rinse-off hair products such as conditioners, and any other form that may be applied to hair.

In one embodiment, the hair care compositions may be provided in the form of a porous, dissolvable solid structure, such as those disclosed in U.S. Patent Application Publication Nos. 2009/0232873; and 2010/0179083, which are incorporated herein by reference in their entirety. As described in these references, such dissolvable solid structure embodiments will typically have a water content well below the at least about 20% aqueous carrier element of certain embodiments described above.

The hair care compositions are generally prepared by conventional methods such as those known in the art of making the compositions. Such methods typically involve mixing of the ingredients in one or more steps to a relatively uniform state, with or without heating, cooling, application of vacuum, and the like. The compositions are prepared such as to optimize stability (physical stability, chemical stability, photostability) and/or delivery of the active materials. The hair care composition may be in a single phase or a single product, or the hair care composition may be in a separate phases or separate products. If two products are used, the products may be used together, at the same time or sequentially. Sequential use may occur in a short period of time, such as immediately after the use of one product, or it may occur over a period of hours or days.

The composition provided by the formula above is made by combining such ingredients in accordance with the method of making provided in this specification.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm”.

All documents cited in the Detailed Description of the Invention are, in relevant part, incorporated herein by reference; the citation of any document is not to be construed as an admission that it is prior art with respect to the present invention. To the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A hair care composition comprising:
A) a material selected from the group consisting of:
(i) a first glyceride copolymer comprising, based on total weight of first glyceride copolymer, from about 3% to about 30% C₁₀₋₁₄ unsaturated fatty acid esters;
(ii) a second glyceride copolymer having formula (I): 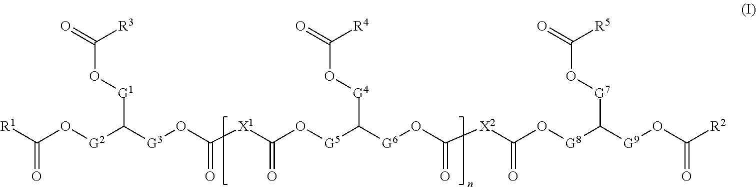
wherein:
each R¹, R², R³, R⁴, and R⁵ in second glyceride copolymer is independently selected from the group consisting of an oligomeric glyceride moiety, a C₁₋₂₄ alkyl, a substituted C₁₋₂₄ alkyl wherein the substituent is one or more —OH moieties, a C₂₋₂₄ alkenyl, or a substituted C₂₋₂₄ alkenyl wherein the substituent is one or more —OH moieties; and/or wherein each of the following combinations of moieties may each independently be covalently linked:
R¹ and R³,
R² and R⁵,
R¹ and an adjacent R⁴,
R² and an adjacent R⁴,
R³ and an adjacent R⁴,
R⁵ and an adjacent R⁴, or
any two adjacent R⁴
such that the covalently linked moieties form an alkenylene moiety;
each X¹ and X² in said second glyceride copolymer is independently selected from the group consisting of a C₁₋₃₂ alkylene, a substituted C₁₋₃₂ alkylene wherein the substituent is one or more —OH moieties, a C₂₋₃₂ alkenylene or a substituted C₂₋₃₂ alkenylene wherein the substituent is one or more —OH moieties;
two of G¹, G², and G³ are —CH₂—, and one of G¹, G², and G³ is a direct bond;
for each individual repeat unit in the repeat unit having index n, two of G⁴, G⁵, and G⁶ are —CH₂—, and one of G⁴, G⁵, and G⁶ is a direct bond, and the values G⁴, G⁵, and G⁶ for each individual repeat unit are independently selected from the values of G⁴, G⁵, and G⁶ in other repeating units;
two of G⁷, G⁸, and G⁹ are —CH₂—, and one of G⁷, G⁸, and G⁹ is a direct bond;
n is an integer from 3 to 250;
with the proviso for each of said second glyceride copolymers at least one of R¹, R², R³, and R⁵, and/or at least one R⁴ in one individual repeat unit of said repeat unit having index n, is selected from the group consisting of: 8-nonenyl; 8-decenyl; 8-undecenyl; 8-dodecenyl; 8,11-dodecadienyl; 8,11-tridecadienyl; 8,11-tetradecadienyl; 8,11-pentadecadienyl; 8,11,14-pentadecatrienyl; 8,11,14-hexadecatrienyl; 8,11,14-octadecatrienyl; 9-methyl-8-decenyl; 9-methyl-8-undecenyl; 10-methyl-8-undecenyl; 12-methyl-8,11-tridecadienyl; 12-methyl-8,11-tetradecadienyl; 13-methyl-8,11-tetradecadienyl; 15-methyl-8,11,14-hexadecatrienyl; 15-methyl-8,11,14-heptadecatrienyl; 16-methyl-8,11,14-heptadecatrienyl; 12-tridecenyl; 12-tetradecenyl; 12-pentadecenyl; 12-hexadecenyl; 13-methyl-12-tetradecenyl; 13-methyl-12-pentadecenyl; and 14-methyl-12-pentadecenyl; and
(iii) optionally, a third glyceride copolymer, which comprises constitutional units formed from reacting, in the presence of a metathesis catalyst, one or more compounds from each of the compounds having the following formulas: 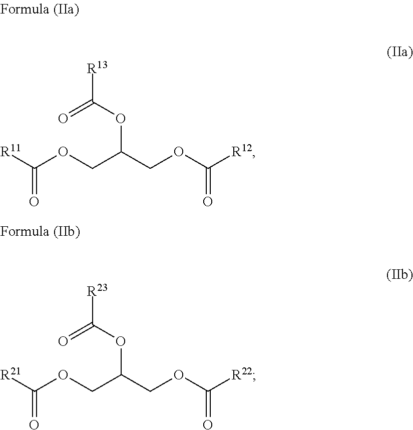
wherein,
each R¹¹, R¹², and R¹³ is independently a C₁₋₂₄ alkyl, a substituted C₁₋₂₄ alkyl wherein the substituent is one or more —OH moieties, a C₂₋₂₄ alkenyl, or a substituted C₂₋₂₄ alkenyl wherein the substituent is one or more —OH moieties with the proviso that at least one of R¹¹, R¹², and R¹³ is a C₂₋₂₄ alkenyl or a substituted C₂₋₂₄ alkenyl wherein the substituent is one or more —OH moieties; and
each R²¹, R²², and R²³ is independently a C₁₋₂₄ alkyl, a substituted C₁₋₂₄ alkyl wherein the substituent is one or more —OH moieties, a C₂₋₂₄ alkenyl, or a substituted C₂₋₂₄ alkenyl wherein the substituent is one or more —OH moieties, with the proviso that at least one of R²¹, R²², and R²³ is 8-nonenyl; 8-decenyl; 8-undecenyl; 8-dodecenyl; 8,11-dodecadienyl; 8,11-tridecadienyl; 8,11-tetradecadienyl; 8,11-pentadecadienyl; 8,11,14-pentadecatrienyl; 8,11,14-hexadecatrienyl; 8,11,14-octadecatrienyl; 9-methyl-8-decenyl; 9-methyl-8-undecenyl; 10-methyl-8-undecenyl; 12-methyl-8,11-tridecadienyl; 12-methyl-8,11-tetradecadienyl; 13-methyl-8,11-tetradecadienyl; 15-methyl-8,11,14-hexadecatrienyl; 15-methyl-8,11,14-heptadecatrienyl; 16-methyl-8,11,14-heptadecatrienyl; 12-tridecenyl; 12-tetradecenyl; 12-pentadecenyl; 12-hexadecenyl; 13-methyl-12-tetradecenyl; 13-methyl-12-pentadecenyl; and 14-methyl-12-pentadecenyl;
wherein the number ratio of constitutional units formed from monomer compounds of formula (IIa) to constitutional units formed from monomer compounds of formula (IIb) is no more than 10:1; and
(iv) mixtures thereof; and
B) a gel matrix phase comprising: (i) from about 0.1% to about 20% of one or more high melting point fatty compounds, by weight of said hair care composition; (ii) from about 0.1% to about 10% of a cationic surfactant system, by weight of said hair care composition; and (iii) at least about 20% of an aqueous carrier, by weight of said hair care composition.

2. The composition of claim 1 wherein the first and second glyceride copolymers have a weight average molecular weight of from about 4,000 g/mol to about 150,000 g/mol.

3. The composition according to claim 1, wherein for the second glyceride copolymer at least one of R¹, R², R³, R⁴, or R⁵ is a C₉₋₁₃ alkenyl.

4. The composition according to claim 1, wherein for the second glyceride copolymer, R¹ is a C₁₋₂₄ alkyl or a C₂₋₂₄ alkenyl.

5. The composition according to claim 1, wherein for the second glyceride copolymer, R² is a C₁₋₂₄ alkyl or a C₂₋₂₄ alkenyl.

6. The composition according to claim 1, wherein for the second glyceride copolymer, R³ is a C₁₋₂₄ alkyl or a C₂₋₂₄ alkenyl.

7. The composition according to claim 1, wherein for the second glyceride copolymer, each R⁴ is independently selected from a C₁₋₂₄ alkyl and a C₂₋₂₄ alkenyl.

8. The composition according to claim 1, wherein for the second glyceride copolymer, R⁵ is a C₁₋₂₄ alkyl or a C₂₋₂₄ alkenyl.

9. The composition according to claim 1, the composition comprising, based on total composition weight, from about 0.1% to about 50% of a glyceride copolymer, selected from the group consisting of said first glyceride copolymer, second glyceride copolymer and mixtures thereof.

10. The composition according to claim 1, wherein each glyceride copolymer has a free hydrocarbon content, based on the weight of glyceride copolymer, of from about 0% to about 5%.

11. The composition of claim 10, wherein each glyceride copolymer has a free hydrocarbon content, based on the weight of glyceride copolymer, of from about 0.1 to about 3%.

12. The composition of claim 11, wherein each glyceride copolymer has a free hydrocarbon content, based on the weight of glyceride copolymer, of from about 0.1 to about 1%.

13. The composition of claim 1, wherein the hair care composition comprises from about 0.5% to about 8%, by weight of the hair care composition, of the cationic surfactant system.

14. The composition of claim 13, wherein the hair care composition comprises from about 0.8% to about 5%, by weight of the hair care composition, of the cationic surfactant system.

15. The composition of claim 1, wherein the hair care composition comprises from about 1% to about 15%, by weight of the hair care composition, of the one or more high melting point fatty compounds.

16. The composition of claim 1, wherein the one or more high melting point fatty compounds comprise from about 14 to about 30 carbon atoms.

17. The composition of claim 16, wherein the high melting point fatty compound is selected from the group consisting of cetyl alcohol, stearyl alcohol, behenyl alcohol, and mixtures thereof.

18. A hair care composition comprising:
A) a first glyceride copolymer and a second glyceride copolymer:
(i) the first glyceride copolymer comprising, based on total weight of first glyceride copolymer, from about 3% to about 30% C₁₀₋₁₄ unsaturated fatty acid esters;
(ii) the second glyceride copolymer having formula (I): 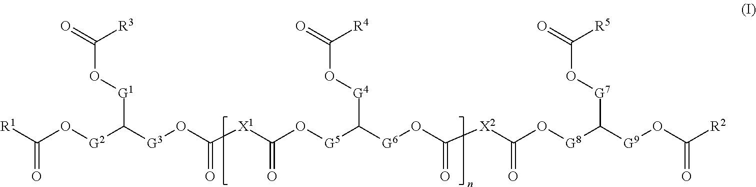
wherein:
each R¹, R², R³, R⁴, and R⁵ in second glyceride copolymer is independently selected from the group consisting of an oligomeric glyceride moiety, a C₁₋₂₄ alkyl, a substituted C₁₋₂₄ alkyl wherein the substituent is one or more —OH moieties, a C₂₋₂₄ alkenyl, or a substituted C₂₋₂₄ alkenyl wherein the substituent is one or more —OH moieties; and/or wherein each of the following combinations of moieties may each independently be covalently linked:
R¹ and R³,
R² and R⁵,
R¹ and an adjacent R⁴,
R² and an adjacent R⁴,
R³ and an adjacent R⁴,
R⁵ and an adjacent R⁴, or
any two adjacent R⁴
such that the covalently linked moieties form an alkenylene moiety; each X¹ and X² in said second glyceride copolymer is independently selected from the group consisting of a C₁₋₃₂ alkylene, a substituted C₁₋₃₂ alkylene wherein the substituent is one or more —OH moieties, a C₂₋₃₂ alkenylene or a substituted C₂₋₃₂ alkenylene wherein the substituent is one or more —OH moieties;
two of G¹, G², and G³ are —CH₂—, and one of G¹, G², and G³ is a direct bond;
for each individual repeat unit in the repeat unit having index n, two of G⁴, G⁵, and G⁶ are —CH₂—, and one of G⁴, G⁵, and G⁶ is a direct bond, and the values G⁴, G⁵, and G⁶ for each individual repeat unit are independently selected from the values of G⁴, G⁵, and G⁶ in other repeating units;
two of G⁷, G⁸, and G⁹ are —CH₂—, and one of G⁷, G⁸, and G⁹ is a direct bond;
n is an integer from 3 to 250;
with the proviso for each of said second glyceride copolymers at least one of R¹, R², R³, and R⁵, and/or at least one R⁴ in one individual repeat unit of said repeat unit having index n, is selected from the group consisting of: 8-nonenyl; 8-decenyl; 8-undecenyl; 8-dodecenyl; 8,11-dodecadienyl; 8,11-tridecadienyl; 8,11-tetradecadienyl; 8,11-pentadecadienyl; 8,11,14-pentadecatrienyl; 8,11,14-hexadecatrienyl; 8,11,14-octadecatrienyl; 9-methyl-8-decenyl; 9-methyl-8-undecenyl; 10-methyl-8-undecenyl; 12-methyl-8,11-tridecadienyl; 12-methyl-8,11-tetradecadienyl; 13-methyl-8,11-tetradecadienyl; 15-methyl-8,11,14-hexadecatrienyl; 15-methyl-8,11,14-heptadecatrienyl; 16-methyl-8,11,14-heptadecatrienyl; 12-tridecenyl; 12-tetradecenyl; 12-pentadecenyl; 12-hexadecenyl; 13-methyl-12-tetradecenyl; 13-methyl-12-pentadecenyl; and 14-methyl-12-pentadecenyl; and
B) a gel matrix phase comprising: (i) from about 0.1% to about 20% of one or more high melting point fatty compounds, by weight of said hair care composition; (ii) from about 0.1% to about 10% of a cationic surfactant system, by weight of said hair care composition; and (iii) at least about 20% of an aqueous carrier, by weight of said hair care composition.

19. The composition of claim 18, wherein the one or more high melting point fatty compounds comprise from about 14 to about 30 carbon atoms and wherein the first glyceride copolymer and the second glyceride copolymer are in the form of particles having an average particle size of from about 0.01 to about 200 μm.

20. The composition of claim 19, wherein the high melting point fatty compound is selected from the group consisting of cetyl alcohol, stearyl alcohol, behenyl alcohol, and mixtures thereof.
